(19) United States
(12) Patent Application Publication
(10) Pub. No.: US 2023/0272484 A1
(43) Pub. Date: Aug. 31, 2023

(54) METHODS OF PROGNOSING, DETERMINING TREATMENT COURSE AND TREATING MULTIPLE MYELOMA

(71) Applicants: Yeda Research and Development Co. Ltd., Rehovot (IL); Ichilov Tech Ltd., Tel-Aviv (IL)

(72) Inventors: Ido AMIT, Rehovot (IL); Assaf WEINER, Rehovot (IL); Mor ZADA, Rehovot (IL); Chamutal BORNSTEIN-OVITS, Rehovot (IL); Shuang-Yin WANG, Rehovot (IL); Yael COHEN, Tel-Aviv (IL)

(73) Assignees: Yeda Research and Development Co. Ltd., Rehovot (IL); Ichilov Tech Ltd., Tel-Aviv (IL)

(21) Appl. No.: 18/142,618

(22) Filed: May 3, 2023

Related U.S. Application Data
(63) Continuation of application No. PCT/IL2021/051306, filed on Nov. 3, 2021.

(30) Foreign Application Priority Data
Nov. 3, 2020 (IL) 278473

(51) Int. Cl. C12Q 1/6886; A61K 38/07; A61K 31/69; A61K 38/13; A61K 31/7076; A61P 35/00

(52) U.S. Cl. CPC C12Q 1/6886; A61K 38/07; A61K 31/69; A61K 38/13; A61K 31/7076; A61P 35/00; C12Q 2600/106; C12Q 2600/118; C12Q 2600/158

(57) ABSTRACT

A method of prognosing a subject diagnosed with multiple myeloma (MM) is provided. Also provided a method of treating a subject diagnosed with MM selected expressing intracellular PPIA and/or RRM2 above a predetermined threshold, the method comprising administering to the subject a therapeutically effective amount of at least one agent which specifically down-regulates activity or expression of PPIA and/or RRM2, thereby treating the subject.

### RELATED APPLICATIONS

This application is a Continuation of PCT Patent Application No. PCT/IL2021/051306 having international filing date of Nov. 3, 2021, which claims the benefit of priority of Israeli Patent Application No. 278473, filed on Nov. 3, 2020. The contents of the above applications are all incorporated by reference as if fully set forth herein in their entirety.

### FIELD AND BACKGROUND OF THE INVENTION

The present invention, in some embodiments thereof, relates to methods of prognosing, determining treatment course and treating multiple myeloma.

Multiple myeloma (MM) is a plasma cells (PCs) malignancy, characterized by expansion of malignant PC in the bone marrow, leading to production of large amount of monoclonal antibodies and ultimately destruction of major organs function^{1,2}. MM remains an incurable malignancy with most patients relapse and die from the disease³. Introduction of novel anti-myeloma drugs and combinations has improved survival in myeloma considerably in the last 15 years, increasing life expectancy from 3-4 years to approximately 7-8 years⁴. Introduction of triplet induction regimens has significantly improved the outcomes of patients with newly diagnosed myeloma (NDMM), with most patients achieving deep and durable responses to their upfront therapy, and favorable survival. In the last decade, carfilzomib, pomalidomide, panobinostat, ixazomib, elotuzumab, daratumumab, isatuximab, and selinexor have been approved by the Food and Drug Administration (FDA) for the treatment of relapsed multiple myeloma and promise to improve outcomes further⁵. Despite these advances, patients with early treatment resistance still suffer poor prognosis. Primary resistance, achieving less than partial response after 4 multi-drug induction cycles, occurs in up to 30% of patients⁶, and is strongly associated with reduced survival^{6,7}. Similarly, early relapse after upfront treatment, in particular within 12-24 months post autologous transplant (ASCT), confers poor outcomes^{8,9,10}, regardless to cytogenetic risk⁸. These ‘induction failure’ patients are typically under-represented in clinical trials and no evidence-based treatment strategy for their management has been established¹¹. These patients comprise a highly unmet need for MM therapies and raise several important challenges. Trials comparing the unique heterogeneity, transcriptional state, and druggable pathways relevant for these patients could drive towards discovery of novel agents in relapsed/refractory myeloma, which are urgently needed.

Ultimately all malignant PC acquire resistance or escape mechanism avoiding even the most advanced treatments^{12,13}. During the progression of the disease, the activation of different signaling cascades contribute to the development of the resistant phenotype¹⁴. These include perturbations in the regulation of cellular stress pathways, hypoxia, PC cell differentiation, apoptosis and autophagy, in combination with mutations and alterations in the expression of the drug targets¹⁵. The acquired resistance is multifactorial including, among other alterations, the levels of expression of proteasome subunits, crosstalk with other proteolytic pathways or overexpression of efflux pumps. Proteasome inhibitors-acquired resistance in MM includes upregulation of the 20S proteasome subunits and downregulation of 19S proteasome subunits^{16,17,18}. Protein homeostasis is also a major player attributing to MM resistance mechanisms. Malignant PC secrete high levels of immunoglobulins with reduced sensitivity to unfolded or misfolded proteins that accumulate in the ER activating ER/UPR stress pathways. Proteasome resistance is associated with perturbation of several genes and proteins in the ER, UPR and proteasome pathways. Acquired proteasome inhibitors (PI) resistance in multiple myeloma has recently been associated with low levels of XBP1 mRNA and ATF6 protein expression^{19,20}. These studies therefore lend support to a therapeutic approach involving the combination of PI with an ER stress-inducing agent to enhance the effectiveness of proteasome inhibition and to overcome PI resistance.

The mechanisms of PC resistance pre- and post-treatment have only been partly resolved. Current technologies for characterizing MM lack the depth, resolution and accuracy needed to molecularly define the malignant cells and pathways driving MM residual disease progression, resistance and relapse^{21,22}. The diversity of patients' response to therapeutic treatments is compound and the biology of MM response involves both cell intrinsic factors such as genomic mutations, epigenetic features, as well as cell extrinsic factors—the microenvironment. The progressive and constant evolving nature of the tumor cells and the microenvironment make it essential to develop new tools for risk stratification and accurate prediction of patient therapeutic response. Thus, new treatment strategies and molecular biomarkers for more successful patient stratification for effective clinical care are needed.

Single-cell technologies are extending the ability to define the complete cellular and molecular makeup across large cohorts of patients, enabling detailed characterization of the tumor cells and their microenvironments^{22,23}. Recent studies demonstrate how single-cell technologies can dramatically advance the way researchers characterize complex immune assemblies and study their spatial organization, clonal distribution, dynamics, pathways, crosstalk, and functions in human disease^{24,25}. Recent studies on MM patients²² demonstrate how scRNA-seq can deepen the understanding of key clinical processes. These studies demonstrate the potential of applying single cell sequencing on clinically defined patients' cohorts for identifying biomarkers for diagnosis and stratification and potential new pathways and targets for therapy.

Additional background art includes:

Drugbank entry: Cladribine for the treatment of MM completed phase I clinical trial [www(dot)drugbank(dot)ca/drugs/DB00242/clinical_trials?conditions=DBCOND0028462%2C DBCOND0051557%2CDBCOND0035632%2CDBCOND0046362%2CDBCOND0028463%2C DBCOND0027951%2CDBCOND0028484%2CDBCOND0028490&phase=1&purpose=treatme nt&status=completed]. Cyclosporine for the treatment of MM completed phase I clinical trial [www(dot)drugbank(dot)ca/drugs/DB00091/clinical_trials?conditions=DBCOND0035632%2C DBCOND0046362%2CDBCOND0028463%2CDBCOND0027951&phase=2&purpose=treatme nt&status=completed].

### SUMMARY OF THE INVENTION

According to an aspect of some embodiments of the present invention there is provided a method of prognosing a subject diagnosed with multiple myeloma (MM), the method comprising determining in plasma cells (PC) of the subject a level of expression of at least one gene of Table A or A* and/or Table B or B*, wherein upregulation in at least one gene of Table A or A* and/or downregulation in at least one gens of Table B or B* as compared to expression of said genes in normal PC is indicative of poor prognosis.

According to some embodiments of the invention, the method further comprises corroborating said prognosis with a Gold standard method.

According to some embodiments of the invention, the method further comprises treating the subject with a treatment modality selected from the group consisting of steroids, chemotherapy, targeted therapy, and stem cell transplant according to said prognosis.

According to an aspect of some embodiments of the present invention there is provided a composition of matter comprising plasma cells of a subject diagnosed with multiple myeloma (MM) and at least one agent which specifically identifies at least one gene of Table A or A* and/or Table B or B*.

According to an aspect of some embodiments of the present invention there is provided a method of determining responsiveness to treatment with Daratumumab, Carfilzomib, Lenalidomide and Dexamethasone (DARA-KRD) in a subject diagnosed with multiple myeloma (MM), the method comprising determining in plasma cells (PC) of the subject a level of expression of at least one gens of Table C and/or Table D, wherein upregulation in at least one gene of Table C and/or downregulation in at least one gene of Table D as compared to expression of said at least one gene in normal PC is indicative of responsiveness to treatment.

According to some embodiments of the invention, the subject exhibits primary resistance to a first line treatment.

According to some embodiments of the invention, the subject is diagnosed with Relapsed/Refractory Multiple Myeloma (RRMM).

According to some embodiments of the invention, the subject exhibits upregulation of said intracellular PPIA and/or RRM2 as compared to expression of same in normal PC.

According to some embodiments of the invention, said subject exhibits early relapse of less than 18 months following improvement.

According to an aspect of some embodiments of the present invention there is provided a method of treating a subject diagnosed with multiple myeloma (MM), the method comprising administering to the subject a therapeutically effective amount of a proteasome inhibitor and at least one agent which specifically down-regulates activity or expression of PPIA and/or RRM2, thereby treating the subject.

According to an aspect of some embodiments of the present invention there is provided a combination comprising a therapeutically effective amount of a proteasome inhibitor and at least one agent which specifically down-regulates activity or expression of PPIA and/or RRM2 for use in treating multiple myeloma (MM) in a subject in need thereof.

According to some embodiments of the invention, said proteasome inhibitor and said at least one agent are in separate formulations.

According to some embodiments of the invention, said proteasome inhibitor and said at least one agent are in a single formulation.

According to an aspect of some embodiments of the present invention there is provided a method of treating a subject diagnosed with MM selected expressing intracellular PPIA and/or RRM2 above a predetermined threshold, the method comprising administering to the subject a therapeutically effective amount of at least one agent which specifically down-regulates activity or expression of PPIA and/or RRM2, thereby treating the subject.

According to an aspect of some embodiments of the present invention there is provided at least one agent which specifically down-regulates activity or expression of PPIA and/or RRM2 for use in treating multiple myeloma (MM) selected expressing intracellular PPIA and/or RRM2 above a predetermined threshold in a subject in need thereof.

According to some embodiments of the invention, the subject exhibits primary resistance to a first line treatment.

According to some embodiments of the invention, said subject exhibits early relapse following an anti MM treatment of less than 18 months.

According to some embodiments of the invention, said proteasome inhibitor is selected from the group consisting of Carfilzomib, Bortezomib and Ixazomib.

According to some embodiments of the invention, the method further comprises determining a level of said intracellular PPIA and/or RRM2 in PC of the subject, wherein upregulation of said intracellular PPIA and/or RRM2 as compared to expression of same in normal PC is indicative of responsiveness to treatment with said proteasome inhibitor and said agent.

According to some embodiments of the invention, said PPIA inhibitor is a pan-cyclophilin inhibitor.

According to some embodiments of the invention, said PPIA inhibitor is CRV431.

According to some embodiments of the invention, said PPIA inhibitor is cyclosporine A (CSa).

According to some embodiments of the invention, said RRM2 inhibitor is Cladribine.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E depict single cell atlas of PC derived from NDMM and PRMM patients. (a) 2D projection showing expression profiles of 51,297 QC-positive single BM PC derived from 60 patients: 11 Control, 15 newly diagnosed MM (NDMM), and 34 Primary Refractory MM (PRMM). (b) 2D projection of a selected set of key plasma cell genes and main MM driver genes over the metacell model (c) Bar plot depicting percentage of healthy-like PC within total collected PC among individual NDMM (N) and PRMM (K) patients, with the total number of collected PC from each patient in the brackets of the X axis labels. (d) Boxplots showing expression of canonical PC genes and common MM driver genes in individual patients (H, control; N, NDMM; K, PRMM). (e) Bar plot depicting malignant PC clonality in individual NDMM and PRMM patients.

FIGS. 1F-I show the sorting strategy of plasma cells and overview single cell data collection from the patients. (f-h) Flow cytometry plots showing sorting strategy (CD38⁺CD138⁺) for plasma cells after doublet exclusion from 3 representative patients. Plots were generated using FlowJo software. (Methods). (i) Schematic diagram showing the statists of patient enrollment to Kydar clinical trial and their current status.

FIGS. 2A-D show that novel MM gene modules define subsets of PRMM patients. (a) Heatmap depicting the z-score of 66 differential genes with a p-value <0.05 between the malignant PC of NDMM and PRMM patients. (b) Dot plots showing average expression of a selected set of highly differential genes between NDMM and PRMM patients, in 4 different patient groups: Healthy, NDMM, PRMM group 1, and PRMM group 2. (c) 2D projection of the same patient groups in panel b over the metacell model. Individual patients are located in the mean coordinates of their corresponding cells. (d) 2D projection of gene module score of the three gene modules in panel a over the metacell model. Individual cells are represented by small dots, while metacells are represented by bigger dots with proportional to the cell number of each metacell.

FIGS. 2E-H show an overview of quality control and data analysis for the single bone marrow plasma cells. (e) Dot plots showing number of reads, number of UMIs, and percentage of cells analyzed per batch of 384 cells (that were pooled for library construction) for all CD38⁺CD138⁺ bone marrow single cells from 66 participants (11 control donors, 15 NDMM patients, and 40 PRMM patients). (Methods). (f) Heat map showing clustering analysis of 3,862 ‘contamination’ cells that pass quality control but do not express key plasma genes. Shown are representative genes of non-PC. (g) Heat map showing clustering analysis of 51,297 PC sorted from all the 66 participants featuring normalized single cell expression levels of a selected set of most variable genes. Clustering is performed using 2,038 genes as features. (h) Heat maps showing clustering analysis of PC from patient KYDAR 21 (left) together with the same number of PC from healthy control group (right) using 1,393 gene features.

FIGS. 3A-M show that gene signatures of PRMM patients can predict clinical response to daratumumab-carfilzomib-lenalidomide-dexamethasone treatment. (a) Kaplan-Meier (KM) analysis of progression-free survival (PFS) comparing patients who achieved partial response (PR) and better and patient who did not achieve PR. The P value is based on a stratified log-rank test. (b) KM analysis as in (a) for overall survival (OS). (c) KM analysis of PFS for patients with double high-risk cytogenetic aberration on FISH analysis compared to single or no high-risk aberration. (d) KM analysis as in (c) for OS. (e) KM analysis of PFS for patients with high module-1 genes score compared to low module-1 score. (f) KM analysis as in (e) for OS. (g) KM analysis comparing PFS of patients with no double hit and low module-1 score (00, blue), patients with double-hit and low module-1 score (10, red), patients with no double hit and high module-1 score (01, green) and patients with double hit and high module-1 score (11, orange). (h) KM analysis as in (g) for OS. (i) Histogram of the log 2 average module-1 gene expression in the MMRF CoMMpass data for 783 newly diagnosed patients (upper panel), 69 patients following first line of treatment (middle panel) and 56 patients after at least two lines of treatments (lower panel). (j) KM analysis for PFS (left) and OS (right) comparing newly diagnosed patients in the CoMMpass data with high module-1 score (red) and patients with low module-1 gene score (blue). (k) KM analysis as in (i) for OS. (1) Multivariate KM analysis for PFS of CoMMpass data with module-1 high and FISH double hit genetic risk. (m) KM analysis as in (1) for OS.

FIGS. 3N-T show differential expression and functional enrichment analyses comparing NDMM and PRMM patients. (n) Scatter plot showing z-score and log 2 fold change of all malignant PC expressed genes in the same patient. X axis showing the expression fold change of the genes (expressed in patient KYDAR 24) compared with PC from healthy control; Y axis showing z-score of the same genes in the same patient. The dot size represents the gene expression in the healthy control PC. (o) Scatter plot showing the −log₁₀ p-value from the Mann-Whitney test and the z-score. (p) The distribution of the z-score from patient KYDAR 24. (q) Volcano plot showing a selected set of differential genes between NDMM and PRMM patients. X axis showing the difference between the average z-score of NDMM patients and PRMM patients; Y axis showing the −log₁₀ p-value from a bootstrap test using t-statistic. (r) Heat map showing the correlation map of all the differential genes identified between the NDMM and PRMM patients. Genes are clustered using hierarchical clustering. (s) Functional enrichment of the 3 gene modules using Metascape. (t) Graphical illustration of non-responder overall PI resistant pathway.

FIGS. 4A-E show molecular pathways of broadly resistant MM patients. (a) Heatmap depicting the z-score of 133 differential genes in NDMM and PRMM patients. The gene list is obtained by comparing the malignant PC of the PRMM responder and non-responder patient groups, with a p-value <0.05. (b) Dot plots showing expression of a selected set of highly differential genes between PRMM responder and non-responder patients. (c) GO enrichment analysis of resistance signature 1 on the left and signature 2 on the right (d) 2D projection of resistance signature scores over the metacell model. Individual cells are represented by small dots, while metacells are represented by bigger dots with proportional to the cell number of each metacell. (e) Box plot showing the response score of individual cells from each patient using a shallow neural network classifier predicting the response to treatment after 4 months. CR: complete response; VGPR: very good partial response; PR: partial response; PD: progressive disease.

FIGS. 4F-I show the clinical response to DARA-KRD treatment. (f) Distribution of international myeloma working group (IMWG) response grades in KYDAR trial. (g) Waterfall plot shows the best overall responses according to the IMWG in MM. (h) and (i) Kaplan-Meir curve showing the progression free survival (panel c) and overall survival (panel d) of PRMM patients treated with DARA-KRD.

FIGS. 5A-F show longitudinal single cell analysis of MM patients pre- and post-treatment. (a) Graphical illustration of clonal evolution and dynamics of malignant PC into 3 main trajectories during DARA-KDR treatment: Sensitive, resistant, and selection. (b) Bar plots depicting longitudinal malignant PC clonality in pre-treatment (baseline) and post-treatment (Cycle 4, 3 months; Cycle 10, 9 months) of individual PRMM patients. Healthy PC are shown in green, while different clones of malignant PC are shown in different shades of grey. Patient outcome at 4 months is indicated right to the patient labels. CR: complete response; VGPR: very good partial response; PR: partial response; PD: progressive disease. (c) Upper part showing 2D projection of malignant PC of patient KYDAR 24, including healthy PC as reference, highlighting all cells (left), cells collected from baseline (middle), and cells collected from Cycle 4 (right). The color of the cells represents healthy PC, and malignant clone 1 and 2. Lower part showing 2D projection of expression of a selected set of differential genes over the metacell model (d) Heatmap showing expression of selected differential genes between the two malignant PC clones of patient KYDAR 24, with healthy PC as reference. (e) The dynamics of CD38 expression in individual patients during DARA-KRD treatment. (f) Box plot showing the response scores of individual cells of each malignant PC clone in individual patients using a shallow neural network classifier predicting the response to treatment after 4 months. CR: complete response; VGPR: very good partial response; PR: partial response; PD: progressive disease.

FIGS. 5G-I show differential expression analysis of DARA-KRD resistant patients. (G) Volcano plot showing a selected set of differential genes between PRMM responder group and PRMM non-responder group. X axis showing the difference between the average z-score of PRMM responder group and PRMM non-responder group; Y axis showing the log₁₀ p-value from a bootstrap test using t-statistic. (H) Scatter plot on the left showing the overlap between gene module 1 in FIG. 2A and resistance signature 1 in FIG. 4A. X axis showing the difference between the average z-score of NDMM patients and PRMM patients; Y axis showing the difference between the average z-score of PRMM responder group and PRMM non-responder group. Scatter plot on the right showing the same as the left one, but for gene module 3 and resistance signature 2. (I) Venn diagram showing the overlap between the PRMM patient groups classified in FIG. 2A and PRMM responder and non-responder groups.

FIGS. 6A-E show that Cyclosporin A, known inhibitor for PPIA synergizes with the proteasome inhibitor carfilzomib (a) Graphical illustration of PRMM non-responder resistant pathway, highlighting PPIA. (b) Dose-response curves for RPMI-8226 and U266 cell lines. cells were incubated with Carfilzomib with or without CsA (3 μg) for 48 hours before cell viability was determined using MTS proliferation assay. Results are shown as the mean+/−SEM (Standard Error of the Mean) of 3 independent experiments. IC₅₀ values for Carfilzomib vs combination therapy is 56 nM and 7.6 nM in RPMI-8226 cell line (Upper panel), and 63 nM and 11 nM in U266 cell line (Lower panel), respectively. P values of the statistical comparison between IC50 values are indicated (***: p<0.001). (c) Immunofluorescence staining of RPMI-8226 cell line post treatment with Carfilzomib or in combination with CsA for 48 h, DAPI (Blue) staining for control live cells, Propidium Iodide (Red) staining for late apoptosis (death), FITC (Green) staining for early apoptosis and merged gate showing all staining. (d) Immunofluorescence analysis and quantification of proliferating cells. Statistical signification was determined using two-way ANOVA and multiple t-tests (***: p<0.001). (e) FACS apoptosis assay using Annexing and DAPI staining for early and late apoptosis, respectively.

FIGS. 6F-H show longitudinal data collection of PRMM patients. (f) Flow cytometry plots showing sorting strategy (CD38⁺CD138⁺) for plasma cells after doublet exclusion, same as in FIG. 1F, but for the samples from patient KYDAR 10 after 4 cycles DARA-KDR treatment. (g) Same as in panel a, but for patient KYDAR 10 after 10 cycles DARA-KDR treatment. (h) Heat map depicting the relative frequency of the immunoglobulin sequences for each clone (including 11 control donor and 15 newly diagnosed patients); left, bottom, immunoglobulin heavy chain constant region (IGHC); middle, immunoglobulin light chain constant region (IGKC and IGLC); right, immunoglobulin light chain variable region (IGKV and IGLV).

FIGS. 7A-D show longitudinal single cell analysis of MM patients (a) Upper part showing 2D projection of malignant PC of patient KYDAR 21, including healthy PC as reference, highlighting all cells (left), cells collected from baseline (middle left), cells collected from Cycle 4 (middle right), cells collected from Cycle 10 (right). The color of the cells represents healthy PC, and malignant clone 1, 2, and 3. Lower part showing 2D projection of expression of a selected set of differential genes over the metacell model. (b) and (c) showing the same as panel (a) but for patients KYDAR 34 and KYDAR 30, respectively and with corresponding samples and clones. (d) The dynamics of S100A11 (left) and S100A10 (right) expression in individual patients during DARA-KRD treatment.

FIGS. 8A-C show that CsA potentiates Carfilzomib and induces cell death in myeloma cell lines. (a) Bar plots showing normalized RNAseq PPIA gene transcription in MM cell lines, highlighted in red lines—RPMI-8226 and U266 cell lines (Methods). (b) Dose-response curves to determine the half maximal inhibitory concentrations (IC50) of CsA in RPMI-8226 and U266 cell lines (Methods). (c) Immunofluorescence staining of U266 cell line post treatment with Carfilzomib or in combination with CsA for 48 h (Methods).

FIGS. 9A-G show that cyclosporin A, known inhibitor for PPIA shown synergistic effect with the proteasome inhibitor carfilzomib. (a) 2D projection showing metacell analysis of scRNA-seq of RPMI-8226 cells during CSA, CFZ, and CFZ+CSA treatment at 4 h and 8 h. Different cell states are indicated by colors. (b) 2D projection showing the distribution of the cells (down sampled to 299 cells) in each sample over the metacell map in panel a. (c) heatmap showing z-score of differentially expressed genes in each cell state. (d) gene ontology enrichment of the 115 up regulated genes and 78 down regulated genes. (e) Heatmap depicting the z-score of resistance signature 1 and 2 genes in NDMM and PRMM patients, together with ex-vivo cells of highly resistant PRMM patient Z01. (f) barplot showing scores of resistance signature 1 in NDMM and PRMM patients, together with resistant PRMM patient Z01. (g) barplot showing Z-scores of PPIA expression in NDMM and PRMM patients, together with resistant PRMM patient Z01.

FIG. 9H is a FACS apoptosis assay using Annexin and DAPI staining for early and late apoptosis, respectively, on ex-vivo cultured patient malignant plasma cells, treated with CFZ, or treated with CFZ+CSA.

FIG. 9I is a barplot showing the quantification of live, early apoptosis, and late apoptosis cells in panel 9H.

### DESCRIPTION OF SPECIFIC EMBODIMENTS OF THE INVENTION

The present invention, in some embodiments thereof, relates to methods of prognosing, determining treatment course and treating multiple myeloma.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Multiple myeloma (MM) is a devastating cancer with a 5 year survival rate of 49%. The disease is often characterized by no early symptoms rendering early diagnosis difficult. Most patients relapse and many later in the course of the disease, become refractory (resistant) to formerly effective treatment.

Whilst conceiving embodiments of the invention and reducing them to practice, the present inventors established a roadmap for combining single cell RNA sequencing (scRNA-seq) with clinical trials to identify new pathways and potential targets for prognosticating and treating MM patients.

Embodiments of the invention are based on a comprehensive scRNA-seq analysis of a prospective clinical trial of patients with inherent resistance including induction non-responsive and early relapsed MM patients, i.e. primary refractory multiple myeloma (PRMM), treated with a combined regimen including daratumumab, carfilzomib, lenalidomide and dexamethasone-DARA-KRD/KYDAR study. Patients bone marrow (BM) aspirates were analyzed longitudinally along treatment, focusing on the different clinical spectra of response. The present inventors comprehensively characterized PCs from the BM of healthy control individuals, newly diagnosed patients (NDMM) and PRMM patients. They found that MM patients, from both the newly diagnosed and refractory groups are defined by similar MM disease drivers. However, analysis of the newly diagnosed MM patients compared to the PRMM group highlighted 3 major gene clusters that are differentially expressed between the groups. These clusters designate several pathways that are perturbed in the relapsed refractory patients, including: hypoxia adaptation, protein folding and mitochondria respiration. The identified gene expression pattern defines a prognostic signature for multiple myeloma progression. A very high clinical predictive value was assigned for the signature. Furthermore, this high-risk signature progressively increased in prevalence in later treatment lines. Since many of the genes within the resistance signature fall into pathways that potentially may synergize with current first line treatment, especially for patients with resistant signatures, the causality of one of the genes in the protein folding pathway, PPIA was determined. Using MM cell lines, and a PPIA small molecule inhibitor (Cyclosporine A), a robust and synergistic tumor killing was demonstrated, achieved by combining Carfilzomib together with Cyclosporine A.

Thus, according to an aspect of the invention there is provided a method of prognosing a subject diagnosed with multiple myeloma (MM), the method comprising determining in plasma cells (PC) of the subject a level of expression of at least one gene of Table A or A* and/or Table B or B*, wherein upregulation in at least 1 gene of Table A or A* (MODULE 1) and/or downregulation in at least 1 gene of Table B or B* (MODULE 3) as compared to expression of said genes in normal PC is indicative of poor prognosis.

TABLE A

| gene name | mean.NDMM | mean.Kydar | ttest.p | module | priority |
|---|---|---|---|---|---|
| COX6C | 0.34964594 | 2.1342976 | 0 | 1 | 1 |
| COX7A2 | 0.02971829 | 1.44781449 | 1.00E−04 | 1 | 1 |
| PPIA | 1.38294898 | 3.2223867 | 1.00E−04 | 1 | 1 |
| NDUFB3 | 0.15300978 | 1.03943202 | 0.0002 | 1 | 1 |
| SNRPE | 0.6903759 | 1.64478047 | 0.0003 | 1 | 1 |
| YWHAQ | 0.23557338 | 1.17332685 | 0.0006 | 1 | 1 |
| UQCR10 | 0.35398292 | 1.12146473 | 0.0006 | 1 | 1 |
| CHCHD2 | −0.0482882 | 0.89021286 | 0.0007 | 1 | 1 |
| SOD1 | 0.49327092 | 1.50866315 | 0.0008 | 1 | 1 |
| RRM2 | 0.11560477 | 0.9888145 | 0.0012 | 1 | 1 |
| STMN1 | 0.02208995 | 1.19688067 | 0.0015 | 1 | 1 |
| NDUFA4 | 0.57796242 | 2.04490419 | 0.0015 | 1 | 1 |
| NDUFB4 | 0.20477955 | 0.99894165 | 0.0025 | 1 | 1 |
| COX6B1 | 0.51365022 | 1.31672226 | 0.0032 | 1 | 1 |
| GNAS | 0.38555648 | 1.94581285 | 0.0036 | 1 | 1 |
| TYMS | 0.20695737 | 0.98071334 | 0.0036 | 1 | 1 |
| ARPC5 | −0.029298 | 0.72237363 | 0.0037 | 1 | 1 |
| PSMA2 | 0.40496235 | 1.18795323 | 0.0043 | 1 | 1 |
| SLC39A8 | 0.28416719 | 1.12625192 | 0.0053 | 1 | 2 |
| GGH | 0.40684344 | 1.29622608 | 0.0065 | 1 | 2 |
| TXN | 1.30096854 | 2.43745129 | 0.0067 | 1 | 2 |
| PSMB4 | 0.87158453 | 1.69299889 | 0.0089 | 1 | 2 |
| YBX1 | −0.0036975 | 0.88083122 | 0.0233 | 1 | 2 |
| UQCRQ | 0.06463558 | 0.84365322 | 0.029 | 1 | 2 |

TABLE A*

| gene name | mean.NDMM | mean.Kydar | ttest.p | module | priority |
|---|---|---|---|---|---|
| PPIA | 1.38294898 | 3.2223867 | 1.00E−04 | 1 | 1 |
| CHCHD2 | −0.0482882 | 0.89021286 | 0.0007 | 1 | 1 |
| SOD1 | 0.49327092 | 1.50866315 | 0.0008 | 1 | 1 |
| RRM2 | 0.11560477 | 0.9888145 | 0.0012 | 1 | 1 |
| STMN1 | 0.02208995 | 1.19688067 | 0.0015 | 1 | 1 |
| TYMS | 0.20695737 | 0.98071334 | 0.0036 | 1 | 1 |
| ARPC5 | −0.029298 | 0.72237363 | 0.0037 | 1 | 1 |
| PSMA2 | 0.40496235 | 1.18795323 | 0.0043 | 1 | 1 |
| TXN | 1.30096854 | 2.43745129 | 0.0067 | 1 | 2 |
| PSMB4 | 0.87158453 | 1.69299889 | 0.0089 | 1 | 2 |
| YBX1 | −0.0036975 | 0.88083122 | 0.0233 | 1 | 2 |

According to an embodiment, the at least one gene is not STMN1 when tested as an individual marker. According to an embodiment, the at least one gene comprises a plurality of gene. According to an embodiment, the plurality of genes is selected form Table A*. According to the an embodiment, the plurality of gene comprises less than 15 genes, lest than 14 genes, less than 13 genes, less than 12 genes, for example, 2-14 genes, 3-14 genes, 4-14 genes, 4-14 genes or 6-14 genes. The present teachings envisage its use when in combination e.g., of less than 15 genes, e.g., 2-14, 4-14, 5-14, 6-14.

TABLE B

| gene name | mean.NDMM | mean.Kydar | ttest.p | module | priority |
|---|---|---|---|---|---|
| UQCRB | 0.64424873 | 1.8784536 | 0.0014 | 2 | 2 |
| SNHG6 | 0.75218244 | 1.75071162 | 0.0015 | 2 | 2 |
| SLC25A3 | 0.17576255 | 0.96924359 | 0.0033 | 2 | 2 |
| MYL6 | −0.1141777 | 0.68197553 | 0.0073 | 2 | 2 |
| CYCS | 0.34340467 | 1.26789882 | 0.01 | 2 | 2 |
| NDUFA1 | −0.0392062 | 0.74209264 | 0.0116 | 2 | 2 |
| UQCRH | 0.50546375 | 1.36729386 | 0.0135 | 2 | 2 |
| EEF1B2 | 1.80831971 | 3.17992191 | 0.015 | 2 | 2 |
| LDHB | 1.16460234 | 2.05839432 | 0.0211 | 2 | 2 |
| NACA | 1.03156766 | 2.21750532 | 0.0261 | 2 | 2 |
| EIF3E | 0.59615547 | 1.44696577 | 0.0283 | 2 | 2 |
| SLC25A5 | 0.66493088 | 1.47051623 | 0.0337 | 2 | 2 |
| LGALS1 | −0.5481013 | 0.5898449 | 0.0351 | 2 | 2 |
| COX7C | 1.64454345 | 2.63002737 | 0.0404 | 2 | 2 |
| BTF3 | 1.48332424 | 2.284007 | 0.0562 | 2 | 2 |
| NPM1 | 1.44693949 | 2.45250409 | 0.0602 | 2 | 2 |
| SUB1 | 0.75158224 | 1.61582771 | 0.0619 | 2 | 2 |

TABLE B*

| gene name | mean.NDMM | mean.Kydar | ttest.p | module | priority | To keep |
|---|---|---|---|---|---|---|
| SLC25A3 | 0.17576255 | 0.96924359 | 0.0033 | 2 | 2 | 1 |
| MYL6 | −0.1141777 | 0.68197553 | 0.0073 | 2 | 2 | 1 |
| CYCS | 0.34340467 | 1.26789882 | 0.01 | 2 | 2 | 1 |
| LDHB | 1.16460234 | 2.05839432 | 0.0211 | 2 | 2 | 1 |
| NPM1 | 1.44693949 | 2.45250409 | 0.0602 | 2 | 2 | 1 |

According to a specific embodiment, the at least one gene is from module 1 in Table A. According to a specific embodiment, the at least one gene is from module 1 in Table A*.

According to a specific embodiment, the at least one gene is from priority 1 in Table A. According to a specific embodiment, the at least one gene is from priority 1 in Table A*.

According to a specific embodiment, the at least one gene is characterized by a p-value <0.005.

As used herein “at least 1” and “at least one” are interchangeable and refer to at least 1, at least 2, at least 3, at least 4, at least 5, at least 6, at least 7, at least 8, at least 9, at least 10, at least 11, at least 12, at least 15, at least 20, at least 25, at least 30 at least 35, at least 40, at least 45, at least 50.

According to another embodiment, the at least 1 is limited by an upper cap of 50, 45, 40, 35, 30, 25, 20, 15, 10 5, 3, 2 genes in the signature.

As used herein, the term “subject” includes mammals, preferably human beings at any age which suffer from MM.

The term “multiple myeloma” as used herein refers to a malignant disorder of plasma cells characterized by uncontrolled and progressive proliferation of a plasma cell clone. The abnormal proliferation of plasma (myeloma) cells causes displacement of the normal bone marrow leading to dysfunction in hematopoietic tissue and destruction of the bone marrow architecture, resulting in progressive morbidity and eventual mortality.

Multiple myeloma is diagnosed according to guidelines provided by the International Myeloma Working Group (IMWG). Subjects with multiple myeloma satisfy CRAB criteria (Calcium elevation, renal insufficiency, anemia and bone abnormalities) (Rajkumar et al., Lancet Oncol 14: e538-48, 2014) and have evidence of measurable secretory disease (measurable M protein in serum or urine; or serum free light chain (SFLC).gtoreq.100 mg/L (involved light chain) and an abnormal serum kappa lambda ratio.

According to a specific embodiment the International Staging System (ISS) is used to classify multiple myeloma. It defines the factors that influence patient survival. The ISS is based on data collected from people with multiple myeloma from around the world. The system has 3 stages based on the measurement of serum albumin and the levels of serum β2-M.
Stage I: β2-M<3.5 mg/L with a serum albumin of 3.5 g/dL or more
Stage II: Either of these 2 criteria:
   β2-M between 3.5 mg/L and 5.5 mg/L
   Albumin <3.5 g/dL
Stage III: β2-M>5.5 mg/L

This system has recently been revised to include serum lactase dehydrogenase (LDH) and high-risk gene abnormalities defined by the FISH test. This is called the revised-ISS (or R-ISS). It is most commonly used to predict prognosis. Higher blood levels of LDH indicate a poorer prognosis. Abnormalities of chromosomes (as defined by the results of cytogenetic testing) in the cancer cells may also show how aggressive the cancer is and influence how the disease progresses.

Recurrent or relapsed myeloma. Myeloma that returns after a period of being in control after treatment is called recurrent myeloma or relapsed myeloma. If there is a recurrence, the cancer may need to be staged again (called re-staging) using one of the systems above.

According to a specific embodiment, the subject is newly diagnosed multiple myeloma (NDMM).

According to a specific embodiment, the subject is diagnosed with MM.

According to a specific embodiment, the subject exhibits symptoms of multiple myeloma (symptomatic).

According to a specific embodiment, the subject does not exhibit symptoms of multiple myeloma (asymptomatic).

According to a specific embodiment, the subject does not exhibit symptoms of multiple myeloma (asymptomatic) with one or more myeloma-defining events.

According to a specific embodiment a myeloma defining event is determined using Gold-standard methods such as baseline bone marrow biopsy, serum free light chain assay (the Freelite® test), and MRI.

As used herein “induction therapy” or “first-line treatment” refers to the initial anti-MM therapy following diagnosis.

The induction therapy is typically designed or selected to:
effectively control the disease, optimally achieving complete remission;
reverse myeloma-related complications;
decrease the risk of early mortality;
be well tolerated with minimal or manageable toxicity;
not interfere with the need for stem cell collection.

A combination of 2-4 (e.g., 2-3, 3) drugs is typically employed.

According to a specific embodiment, the combination includes Velcade® (bortezomib), Revlimid® (lenalidomide), and low-dose dexamethasone (VRd).

Other induction therapies include, but are not limited to, the following:
Velcade (bortezomib), Cytoxan® (cyclophosphamide), and dexamethasone (VCD or CyBorD)
Velcade (bortezomib), Thalomid® (thalidomide), and dexamethasone (VTD)
Revlimid (lenalidomide) and dexamethasone (Rd)
Velcade (bortezomib) and dexamethasone (Vd)
VRd Lite (reduced dose and schedule of Velcade, Revlimid, and dexamethasone).

Patients received a bortezomib-based induction and either failed to achieve a timely response (<6, 5, 4 months=PRMM) or progressed early (<18 months)=RRMM.

According to a specific embodiment, the subject exhibits primary resistance to treatment of less than 6 months, 5 months or 4 months following treatment initiation.

According to a specific embodiment, the subject exhibits early relapse less than 18 months following improvement.

According to a specific embodiment, the subject received a bortezomib-based induction and either failed to achieve a timely response (<4 months, e.g., cohort A) or progressed early (<18 months e.g., cohort B).

As used herein “prognosing” or “predicting prognosis” refers to predicting survival of a subject diagnosed with MM, or in other words, risk for for death or for disease progression compared to subjects which are not characterized by the gene expression pattern according to some embodiments of the invention (also referred to as “signature”).

According to a specific embodiment, gene expression predicts poor prognosis. A rough classification of prognosis is provided as follows:
good (low risk) prognosis—likely to survive 8 to 10 years;
intermediate prognosis—likely to survive 5 years;
poor prognosis—likely to survive less than 2 years.

Once prognosis has been made, the subject may be directed to further tests to corroborate the findings, such as Gold standard methods. Examples include, but are not limited, to:

Beta-2-microglobulin—Beta-2-microglobulin is a protein found on the surface of myeloma cells that plays a role in the immune response. A higher level of beta-2-microglobulin predicts a poor prognosis. The level of this protein goes up if:
the number of myeloma cells goes up
there is kidney damage
Albumin—Albumin is the main protein in plasma that helps to maintain blood volume. A higher level of albumin predicts a better prognosis.
Lactate dehydrogenase—A higher level of LD predicts a poorer prognosis.
Creatinine—A high creatinine level have a poorer prognosis.
Chromosome changes—Chromosomal aberrations are linked to a poorer prognosis, including but not limited:
a deletion in chromosome 13;
a 17p deletion;
a translocation in chromosome 14;
chromosomal amplification;
Chromosomal aberrations are typically determined by FISH or sequencing.
Kidney function;

Plasma cell labelling index—The plasma cell labelling index (PCLI) measures how fast myeloma cells are growing in a sample of cells removed from the bone marrow. A high PCLI predicts that the myeloma cells are growing quickly and is linked to a poor prognosis.

According to some embodiments of the invention, the method further comprising informing the subject of the predicted prognosis of the subject.

As used herein the phrase “informing the subject” refers to advising the subject that based on the determined prognosis, the subject should seek further corroboration of the prognosis and optionally suitable treatment regimen (e.g., steroids, chemotherapy, targeted therapy, and stem cell transplant according to said prognosis).

Once prognosis is determined, the results can be recorded in the subject's medical file, which may assist in selecting a treatment regimen and/or determining prognosis of the subject.

As mentioned, the prediction of the prognosis of a subject can be used to select the treatment regimen of a subject and thereby treat the subject in need thereof.

Determination of the gene expression pattern in the plasma cells can be done using methods which are well known in the art and are described hereinbelow and in the Examples section which follows.

Plasma cells occurrence is increased in the bone marrow of MM patients. Hence a bone marrow biopsy or aspiration is typically employed to check the percentage and optionally appearance of the plasma cells in the bone marrow. The bone marrow tissue and optionally the aspirate are tested typically by immunohustochemistry, flow cytometry, cytogenetics and/or Fluorescent in situ hybridization (FISH).

As used herein “normal PC” serve as control for gene expression. They are retrieved (or gene expression information pertaining to same) from subjects which are considered healthy and are not diagnosed with MM (should be ascertained).

As mentioned, upregulation in at least one gene of Table A (MODULE 1) and/or downregulation in at least one gene of Table B (MODULE 3) as compared to expression of said genes in normal PC is indicative of poor prognosis.

In other embodiments, upregulation in at least one gene of Table A* (MODULE 1) and/or downregulation in at least one gene of Table B* (MODULE 3) as compared to expression of said genes in normal PC is indicative of poor prognosis.

According to another aspect of the invention, there is provided a method of determining responsiveness to treatment with Daratumumab, Carfilzomib, Lenalidomide and Dexamethasone (DARA-KRD) in a subject diagnosed with multiple myeloma (MM), the method comprising determining in plasma cells (PC) of the subject a level of expression of at least one gene of Table C and/or Table D, wherein upregulation in at least one gene of Table C and/or downregulation in at least one gene of Table D as compared to expression of said genes in normal PC is indicative of responsiveness to treatment.

TABLE C

| | | | | Res- |
|---|---|---|---|---|
| Gene name | mean.Res | mean.nRes | ttest.p | NonRes |
| ITGB7 | 0.94350413 | 5.00538192 | 0.0084 | 4.06187779 |
| S100A4 | −0.9617219 | 2.24050551 | 0.0002 | 3.2022274 |
| TUBA1B | 0.66352357 | 3.8606696 | 0 | 3.19714603 |
| STMN1 | 0.46572942 | 3.57117036 | 0 | 3.10544094 |
| LILRB4 | 0.89152659 | 3.8948655 | 0.001 | 3.00333891 |
| COX6C | 1.53474279 | 4.18060417 | 0.0003 | 2.64586138 |
| GAPDH | 2.18373229 | 4.72325262 | 0.0032 | 2.53952033 |
| PPIA | 2.75007002 | 5.23215483 | 0.0053 | 2.48208481 |
| RAN | 1.02313239 | 3.48852781 | 1.00E−04 | 2.46539542 |
| LDHA | 1.40351201 | 3.85238117 | 1.00E−04 | 2.44886916 |
| S100A11 | 0.03043745 | 2.31245691 | 0.0106 | 2.28201946 |
| RRM2 | 0.54803011 | 2.66117409 | 0.0004 | 2.11314398 |
| H2AFZ | 0.49009719 | 2.58537289 | 1.00E−04 | 2.09527571 |
| TYMS | 0.52546239 | 2.46181188 | 1.00E−04 | 1.93634948 |
| HMGB2 | −0.1019963 | 1.83352196 | 0 | 1.93551828 |
| ENO1 | −0.0704901 | 1.86209212 | 0 | 1.93258219 |
| LY6E | 0.41160749 | 2.23580911 | 0.0067 | 1.82420161 |
| S100A10 | 0.04244666 | 1.76840937 | 0.0033 | 1.72596271 |
| LDHB | 1.70924286 | 3.4154731 | 0.0037 | 1.70623024 |
| PDCD5 | 1.02010679 | 2.72429032 | 1.00E−04 | 1.70418353 |
| YBX1 | 0.71456859 | 2.36267888 | 0.0014 | 1.64811028 |
| PEBP1 | −0.3418108 | 1.30356168 | 0.0116 | 1.64537247 |
| GGH | 0.91003049 | 2.53956031 | 0.0023 | 1.62952983 |
| LAMP5 | 1.59846877 | 3.19707468 | 0.0522 | 1.59860591 |
| NDUFB9 | 0.90726387 | 2.4795527 | 0.0002 | 1.57228883 |
| PSMB4 | 1.28310622 | 2.84130422 | 0.0021 | 1.558198 |
| HDGF | 0.53046077 | 2.088339 | 0 | 1.55787823 |
| TOP2A | 0.11047357 | 1.66056173 | 0.0009 | 1.55008816 |
| TUBB | 1.00265264 | 2.54237205 | 0.002 | 1.53971941 |
| COX6A1 | 0.03408368 | 1.56897324 | 1.00E−04 | 1.53488956 |
| CCT3 | 0.61886954 | 2.13104877 | 0.0013 | 1.51217923 |
| PTMA | 0.81683487 | 2.31872299 | 0.013 | 1.50188811 |
| UCHL1 | 0.84276077 | 2.26048445 | 0.0729 | 1.41772368 |
| TXN | 2.23517381 | 3.64600155 | 0.0078 | 1.41082774 |
| ACTG1 | 0.12039666 | 1.50670479 | 0.0287 | 1.38630813 |
| NME2 | 1.85472012 | 3.22764195 | 0.0051 | 1.37292183 |
| PTMAP4 | 0.99419543 | 2.36004431 | 0.0104 | 1.36584888 |
| FAM26F | 0.3981314 | 1.75749429 | 0.0098 | 1.35936289 |
| COX7B | 1.27320352 | 2.62595113 | 0.0004 | 1.35274761 |
| ARPC1B | 0.35486884 | 1.70248812 | 0.002 | 1.34761928 |
| IFI30 | −0.0291872 | 1.31732712 | 0.0217 | 1.34651431 |
| Clorf43 | 0.70741259 | 2.0405791 | 0.0014 | 1.33316651 |
| SF3B2 | −0.0429791 | 1.28070159 | 0.0008 | 1.3236807 |
| SMC4 | 0.86975154 | 2.18167461 | 0.0007 | 1.31192306 |
| MYL6 | 0.39599219 | 1.70606088 | 0.008 | 1.31006868 |
| PAGE5 | 0.29506898 | 1.58525866 | 0.0206 | 1.29018968 |
| HRASLS2 | 0.10630809 | 1.39123996 | 0.0022 | 1.28493187 |
| NME1 | 0.98194513 | 2.2618813 | 0.0011 | 1.27993617 |
| SNRPE | 1.34541593 | 2.62258873 | 0.0025 | 1.2771728 |
| TAGLN2 | 0.44277321 | 1.70425531 | 0.0039 | 1.2614821 |
| TSEN15 | 0.30968096 | 1.57090421 | 1.00E−04 | 1.26122325 |
| SSBP1 | 0.99383943 | 2.24708956 | 0.0042 | 1.25325014 |
| SNHG6 | 1.48254328 | 2.71724897 | 0.0202 | 1.23470568 |
| NPM1 | 2.20119061 | 3.42019115 | 0.02 | 1.21900055 |
| NDUFS6 | 0.11080144 | 1.31019183 | 0.0024 | 1.19939039 |
| TPI1 | 0.57291127 | 1.76184187 | 0.0062 | 1.1889306 |
| SOD1 | 1.16014887 | 2.33301207 | 0.0027 | 1.1728632 |
| CBX3 | 0.50009951 | 1.66682256 | 0.0078 | 1.16672305 |
| PAGE2B | 0.02142666 | 1.18653802 | 0.0113 | 1.16511135 |
| SNRPD2 | 1.17267934 | 2.32350673 | 0.0052 | 1.15082739 |
| PFN1 | 0.72170741 | 1.866461 | 0.0177 | 1.14475359 |
| PAGE2 | 0.01583656 | 1.14511368 | 0.0184 | 1.12927712 |
| CCT6A | 0.75837534 | 1.88458538 | 0.0024 | 1.12621004 |
| ASS1 | 0.64080296 | 1.74935042 | 0.032 | 1.10854746 |
| SNRPF | 0.53016914 | 1.62584929 | 0.0107 | 1.09568014 |
| MYC | 0.84342642 | 1.9322802 | 0.0313 | 1.08885378 |
| MAFB | 0.00354351 | 1.08765439 | 0.0104 | 1.08411088 |
| EIF4EBP1 | 0.39960617 | 1.48361362 | 0.0006 | 1.08400745 |
| COX8A | 1.08232902 | 2.1538119 | 0.0013 | 1.07148288 |
| PSMD4 | 0.60589116 | 1.66485873 | 0.0015 | 1.05896758 |
| PLAC1 | 0.07287319 | 1.12857547 | 0.0618 | 1.05570228 |
| MTHFD2 | 0.70112322 | 1.75092689 | 0.0233 | 1.04980367 |
| NDUFB2 | 0.70386347 | 1.75135062 | 0.0389 | 1.04748715 |
| TLR4 | 0.23652552 | 1.27553342 | 0.0238 | 1.0390079 |
| SCAMP3 | 0.2884123 | 1.32677537 | 0.0013 | 1.03836307 |
| FKBP5 | 0.45793202 | 1.47803031 | 0.0175 | 1.02009829 |
| HIST1H1C | 0.35522412 | 1.37045853 | 0.0334 | 1.01523441 |
| CFL1 | 0.95421902 | 1.96034946 | 0.0233 | 1.00613044 |

TABLE D

| | | | | Res- |
|---|---|---|---|---|
| Gene name | mean.Res | mean.nRes | ttest.p | NonRes |
| CD63 | −0.7405702 | −1.7635172 | 0.0808 | −1.022947 |
| SELENOS | −1.3359251 | −2.3685071 | 0.0209 | −1.032582 |
| CCDC69 | −1.1730363 | −2.2321293 | 0.0016 | −1.059093 |
| EIF4A2 | 0.09527033 | −1.0084784 | 0.0079 | −1.1037487 |
| FNDC3B | −1.7665665 | −2.9291673 | 6.00E−04 | −1.1626008 |
| CPNE5 | −0.8286332 | −2.1719747 | 0.002 | −1.3433414 |
| SELENOK | −1.4187692 | −2.7836164 | 0.015 | −1.3648472 |
| RRBP1 | −0.60002 | −1.9795652 | 0.0473 | −1.3795452 |
| FNDC3A | −0.7944218 | −2.1988845 | 0 | −1.4044627 |
| ITM2C | −2.3675233 | −3.7835169 | 0.1592 | −1.4159936 |
| PNRC1 | −0.7466023 | −2.220077 | 0.0014 | −1.4734747 |
| OS9 | −1.4230937 | −2.910181 | 0.0015 | −1.4870873 |
| HYOU1 | −0.6463976 | −2.1647825 | 0.0015 | −1.5183849 |
| FKBP2 | −0.2604546 | −1.79774 | 0.0483 | −1.5372853 |
| NUCB2 | −1.4746719 | −3.0161269 | 0.1076 | −1.541455 |
| SEL1L | −2.093937 | −3.6404915 | 0.0206 | −1.5465545 |
| ERLEC1 | −2.4012558 | −3.9705572 | 0.0066 | −1.5693013 |
| CD27 | −2.8019267 | −4.3920951 | 0.0081 | −1.5901683 |
| FCRL5 | −1.7449332 | −3.3488481 | 0.0063 | −1.6039149 |
| SELENOM | −1.1177403 | −2.7320765 | 0.009 | −1.6143362 |
| ATF4 | 0.9589607 | −2.6023442 | 0.001 | −1.6433835 |
| SDF2L1 | −0.3133111 | −1.9570838 | 0.0016 | −1.6437727 |
| CRELD2 | −1.2199366 | −2.8834137 | 0.0011 | −1.6634771 |
| FAM46C | −1.4177267 | −3.0868178 | 0.0298 | −1.6690911 |
| BASP1 | 0.58941074 | −1.0856783 | 0.0101 | −1.6750891 |
| TAPBP | −1.275194 | −2.9716676 | 6.00E−04 | −1.6964736 |
| SYVN1 | −0.3342735 | −2.03218 | 0 | −1.6979065 |
| HDLBP | −2.2555984 | −3.9608123 | 3.00E−04 | −1.7052139 |
| SLAMF7 | −2.1039536 | −3.8174738 | 0.0416 | −1.7135202 |
| POU2AF1 | −0.6003171 | −2.3199988 | 0.001 | −1.7196817 |
| FBXW7 | −1.5654047 | −3.3037373 | 1.00E−04 | −1.7383326 |
| PECAM1 | −2.3379017 | −4.0763197 | 0.0169 | −1.7384181 |
| EEF1D | 0.5775849 | −1.1729014 | 0.0097 | −1.7504863 |
| ST6GAL1 | −1.3491662 | −3.1354283 | 0.004 | −1.786262 |
| MDK | −0.0684799 | −1.8603063 | 4.00E−04 | −1.7918264 |
| TAPBPL | −1.6671138 | −3.4812824 | 8.00E−04 | −1.8141685 |
| HERPUD1 | −1.7865623 | −3.6728665 | 0.0242 | −1.8863043 |
| FCGR2B | 0.47826645 | −1.4383974 | 0 | −1.9166639 |
| FKBP11 | −3.2118444 | −5.1780766 | 0.0198 | −1.9662322 |
| NOP53 | 0.05470011 | −1.9499246 | 0.0034 | −2.0046248 |
| QPCT | 0.96605184 | −1.0512929 | 0.0124 | −2.0173447 |
| CTSS | −1.2140747 | −3.252847 | 0.0022 | −2.0387723 |
| BTG2 | −0.4711286 | −2.5567673 | 0.0035 | −2.0856387 |
| ADA2 | −1.6253581 | −3.8390175 | 0.001 | −2.2136594 |
| CD79A | −1.4890776 | −3.703897 | 0.0025 | −2.2148194 |
| SEL1L3 | −2.6181526 | −5.0532912 | 0.0076 | −2.4351386 |
| XBP1 | −4.5976772 | −7.2064363 | 0.0106 | −2.6087591 |
| SEC62 | −1.9066371 | −4.6494937 | 0 | −2.7428566 |
| BLOC1S5- | −4.1943905 | −7.0276392 | 0.0309 | −2.8332487 |
| TXNDC5 | | | | |
| HLA-DOB | −0.2084085 | −3.0953228 | 0 | −2.8869144 |
| ITM2B | −1.0320467 | −4.172361 | 2.00E−04 | −3.1403143 |
| DERL3 | −1.791932 | −4.9495749 | 9.00E−04 | −3.1576429 |
| HLA-E | −0.2680453 | −3.5367706 | 4.00E−04 | −3.2687253 |
| TPT1 | −0.1326641 | −3.5460904 | 0.0088 | −3.4134262 |
| CD74 | −2.4464482 | −6.3490881 | 0.0068 | −3.90264 |
| SSR4 | −3.172336 | −8.026047 | 4.00E−04 | −4.853711 |

According to a specific embodiment, the at least one gene is not STMN1.

According to a specific embodiment, the at least one gene is from priority 1

According to a specific embodiment, the at least one gene is characterized by a p-value <0.005.

As used herein “at least 1” refers t at least 1, at least 2, at least 3, at least 4, at least 5, at least 6, at least 7, at least 8, at least 9, at least 10, at least 11, at least 12, at least 15, at least 20, at least 25, at least 30 at least 35, at least 40, at least 45, at least 50.

According to another embodiment, the at least 1 is limited by an upper cap of 50, 45, 40, 35, 30, 25, 20, 15, 10 5, 3, 2 genes in the signature.

According to another embodiment, the at least 1 is limited by an upper cap of less than 15 genes in the signature.

According to another embodiment, the at least 1 is limited by an upper cap of less than 10 genes in the signature.

According to another embodiment, the at least 1 is in the range of 2-14, 3-14, 4-14, 5-14, 6-14, 7-14, 8-14, 9-14, 10-14, 11-14, 6-14, 6-12, 6-10.

According to another embodiment, the at least 1 is in the range of 2-12, 3-12, 4-12, 5-12, 6-12, 7-12, 8-12, 9-12, 10-12.

According to another embodiment, the at least 1 is in the range of 2-10, 3-10, 4-10, 5-10, 6-10, 7-10, 8-10, 9-10.

As used herein “determining responsiveness” refers to an ex vivo method for determining the likelihood of a subject to benefit from a treatment as described herein. Responsiveness is determined when any of the at least one gene exhibits a statistically significant gene expression as compared to same in normal PC.

As used herein “DARA-KRD” refers to a quadruple combination of Daratumumab, Carfilzomib, Lenalidomide and Dexamethasone.

A typical regimen with a quadruple regimen comprises: Daratumumab 16 mg/Kg weekly during cycles 1-2, q14 days during cycles 3-6, thereafter monthly (1st dose cycle 1 may be split over 2 days); Once-weekly intravenous (IV) Carfilzomib on days 1, 8, 15, of cycle numbers 1-9 and Days 1 and 15 only of cycle numbers 10-18, at a dose of 20 mg/m² on day 1 of cycle 1; at dose of 56 mg/m² on all subsequent once weekly dosing days, alongside concomitant treatment with twice-weekly IV or oral dexamethasone 20 mg administered on Days 1-2, 8-9, 15-16, and 22-23 of a 28-day cycle, for cycles 1-2 followed by weekly 20 mg dexamethasone on subsequent cycles; and oral Lenalidomide 25 mg, administered on days 1-21 of a 28-day cycle (D-KRd). Frail patients (as per IMWG recommendations;⁵) receive Lenalidomide dose adjustment to 15 mg, and dexamethasone at 10 mg×2/week cycles 1-2 followed by 10 mg/week for subsequent cycles. The quadruple regimen is administered for 18 cycles, followed by long-term follow-up in which patients receive standard of care treatment (lenalidomide maintenance or lenalidomide daratumumab). Pre-infusion medications include diphenhydramine (or equivalent), acetaminophen, and montelukast).

Methods of determining gene expression profiles can be performed at the RNA or protein level.

Below is a more detailed description of methods that can be used to analyze expression of a plurality of genes on the single cell level.

Methods of Analyzing and/or Quantifying RNA

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Single Cell Transcriptome Analysis

This method relies on sequencing the transcriptome of a single cell. In one embodiment a high-throughput method is used, where the RNAs from different cells are tagged individually, allowing a single library to be created while retaining the cell identity of each read. The method can be carried out a number of ways—see for example US Patent Application No. 20100203597 and US Patent Application No. 20180100201, the contents of which are incorporated herein by reference.

One particular method for carrying out single cell transcriptome analysis is detailed herein below.

Cells are typically aliquoted into wells such that only one cell is present per well. Cells are treated with an agent that disrupts the cell and nuclear membrane making the RNA of the cell accessible to sequencing reactions.

According to one embodiment, the RNA is amplified using the following in vitro transcription amplification protocol: (Step 1) contacting the RNA of a single cell with an oligonucleotide comprising a polydT sequence at its terminal 3′ end, a T7 RNA polymerase promoter sequence at its terminal 5′ end and a barcode sequence positioned between the polydT sequence and the RNA polymerase promoter sequence under conditions that allow synthesis of a single stranded DNA molecule from the RNA, wherein the barcode sequence comprises a cell barcode and a molecular identifier; The polydT oligonucleotide of this embodiment may optionally comprise an adapter sequence required for sequencing.

RNA polymerase promoter sequences are known in the art and include for example T7 RNA polymerase promoter sequence.

Preferably the polydT sequence comprises at least 5 nucleotides. According to another embodiment the polydT sequence is between about 5 to 50 nucleotides, more preferably between about 5-25 nucleotides, and even more preferably between about 12 to 14 nucleotides.

The barcode sequence is useful during multiplex reactions when a number of samples are pooled in a single reaction. The barcode sequence may be used to identify a particular molecule, sample or library. The barcode sequence is attached 5′ end of polydT sequence and 3′ of the T7 RNA polymerase sequence. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides.

In one embodiment, the barcode sequence is used to identify a cell type, or a cell source (e.g. a patient).

Molecular identifiers are useful to correct for amplification bias, which reduces quantitative accuracy of the method. The molecular identifier comprises between 4-20 bases. The molecular identifier is of a length such that each RNA molecule of the sample is catalogued (labeled) with a molecular identifier having a unique sequence.

Following annealing of a primer (e.g. polydT primer) to the RNA sample, an RNA-DNA hybrid may be synthesized by reverse transcription using an RNA-dependent DNA polymerase. Suitable RNA-dependent DNA polymerases for use in the methods and compositions of the invention include reverse transcriptases (RTs). RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules. However, in some instances, it is preferable to employ a RT which lacks or has substantially reduced RNase H activity.

RTs devoid of RNase H activity are known in the art, including those comprising a mutation of the wild type reverse transcriptase where the mutation eliminates the RNase H activity. Examples of RTs having reduced RNase H activity are described in US20100203597. In these cases, the addition of an RNase H from other sources, such as that isolated from E. coli, can be employed for the formation of the single stranded cDNA. Combinations of RTs are also contemplated, including combinations of different non-mutant RTs, combinations of different mutant RTs, and combinations of one or more non-mutant RT with one or more mutant RT.

Examples of suitable enzymes include, but are not limited to AffinityScript from Agilent or Superscript III from Invitrogen. Preferably the reverse transcriptase is devoid of terminal Deoxynucleotidyl Transferase (TdT) activity.

Additional components required in a reverse transcription reaction include dNTPS (dATP, dCTP, dGTP and dTTP) and optionally a reducing agent such as Dithiothreitol (DTT) and MnCl₂.

The polydT oligonucleotide may be attached to a solid support (e.g. beads) so that the cDNA which is synthesized may be purified.

Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

(Step 2): Once cDNA is generated, the cDNA may be pooled from cDNA generated from other single cells (using the same method as described herein above).

The sample may optionally be treated with an enzyme to remove excess primers, such as exonuclease I. Other options of purifying the single stranded DNA are also contemplated including for example the use of paramagnetic microparticles. This may be carried out following or prior to sample pooling.

(Step 3): Second strand synthesis.

Second strand synthesis of cDNA may be effected by incubating the sample in the presence of nucleotide triphosphates and a DNA polymerase. Commercial kits are available for this step which include additional enzymes such as RNAse H (to remove the RNA strand) and buffers. This reaction may optionally be performed in the presence of a DNA ligase. Following second strand synthesis, the product may be purified using methods known in the art including for example the use of paramagnetic microparticles.

(Step 4): Following synthesis of the second strand of the cDNA, RNA may be synthesized by incubating with a corresponding RNA polymerase. Commercially available kits may be used such as the T7 High Yield RNA polymerase IVT kit (New England Biolabs).

(Step 5): Prior to fragmentation of the amplified RNA, the DNA may be removed using a DNAse enzyme. The RNA may be purified as well prior to fragmentation. Fragmentation of the RNA may be carried out as known in the art. Fragmentation kits are commercially available such as the Ambion fragmentation kit.

(Step 6): The amplified and fragmented RNA is now labeled on its 3′ end. For this a ligase reaction is performed which essentially ligates single stranded DNA (ssDNA) to the RNA. Other methods of labeling the amplified and fragmented RNA are described in US Application No. 20170137806, the contents of which are incorporated herein by reference. The single stranded DNA has a free phosphate at its 5′end and optionally a blocking moiety at its 3′end in order to prevent head to tail ligation. Examples of blocking moieties include C3 spacer or a biotin moiety. Typically, the ssDNA is between 10-50 nucleotides in length and more preferably between 15 and 25 nucleotides.

(Step 7): Reverse transcription is then performed using a primer that is complementary to the primer used in the preceding step. The library may then be completed and amplified through a nested PCR reaction.

(Step 8): Amplification

Once the adapter polynucleotide of the present invention is ligated to the single stranded DNA (i.e. further to extension of the single stranded DNA), amplification reactions may be performed.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the adapter-extended DNA described herein together with any additional amplification reaction reagents under conditions which allow amplification of the target sequence.

The terms “forward primer” and “forward amplification primer” are used herein interchangeably, and refer to a primer that hybridizes (or anneals) to the target (template strand).

The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphosphate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favor solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs).

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Amplification products obtained using primers of the present invention may be detected using agarose gel electrophoresis and visualization by ethidium bromide staining and exposure to ultraviolet (UV) light or by sequence analysis of the amplification product.

According to one embodiment, the amplification and quantification of the amplification product may be effected in real-time (qRT-PCR).

(Step 9): Sequencing

Methods for sequence determination are generally known to the person skilled in the art. Preferred sequencing methods are next generation sequencing methods or parallel high throughput sequencing methods e.g. Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

It will be appreciated that methods which rely on microfluidics can also be used to carry out single cell transcriptome analysis.

Thus, a combination of molecular barcoding and emulsion-based microfluidics to isolate, lyse, barcode, and prepare nucleic acids from individual cells in high-throughput may be used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode about 10,000-100,000 cells. In this regard there can be a single-cell sequencing library which may comprise: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells: and preparing and sequencing a single composite RNA-Seq library, as described herein above. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2, all the contents and disclosure of each of which are herein incorporated by reference in their entirety.

Methods of Detecting Expression and/or Activity of Proteins

Expression and/or activity level of proteins expressed in the cells of the cultures of some embodiments of the invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to a specific embodiment, the gene expression is determined by transcriptome analysis.

According to a specific embodiment, the gene expression is determined by a single cell transcriptome analysis as described above.

According to a specific embodiment, the responsiveness or prognosis are determined by the level of at least one (e.g., 2, 3, 4, 5, 6, 7 or 8) but not more than 10 genes (markers) (e.g., 1-10, 2-10, 3-10, 4-10, 5-10, 6-10, 7-10, 8-10, 1-5, 2-5, 3-5, 4-5, 1-8, 2-8, 3-8, 4-8, 5-8). Positive (+) and negative (−) expression are determined as known in the art.

During the performance of the methods of determining responsiveness/prognosis as described herein, naturally the biological sample is combined (following some processing, e.g., cell lysis, RNA purification, protein purification and the like) with the agents which determine the level of expression of the above-mentioned genes (such a composition can also be used as control).

Thus, according to an aspect of the invention there is provided a composition of matter comprising plasma cells of a subject diagnosed with multiple myeloma (MM) and at least one agent which specifically identifies at least one gene of Table A or A* and/or Table B or B* (MODULE 1 AND MODULE 3 RESPECTIVELY and optionally MODULE 2).

According to an alternative embodiment, there is provided a composition of matter comprising plasma cells of a subject diagnosed with multiple myeloma (MM) and at least one agent which specifically identifies at least one gene of Table C and/or Table D.

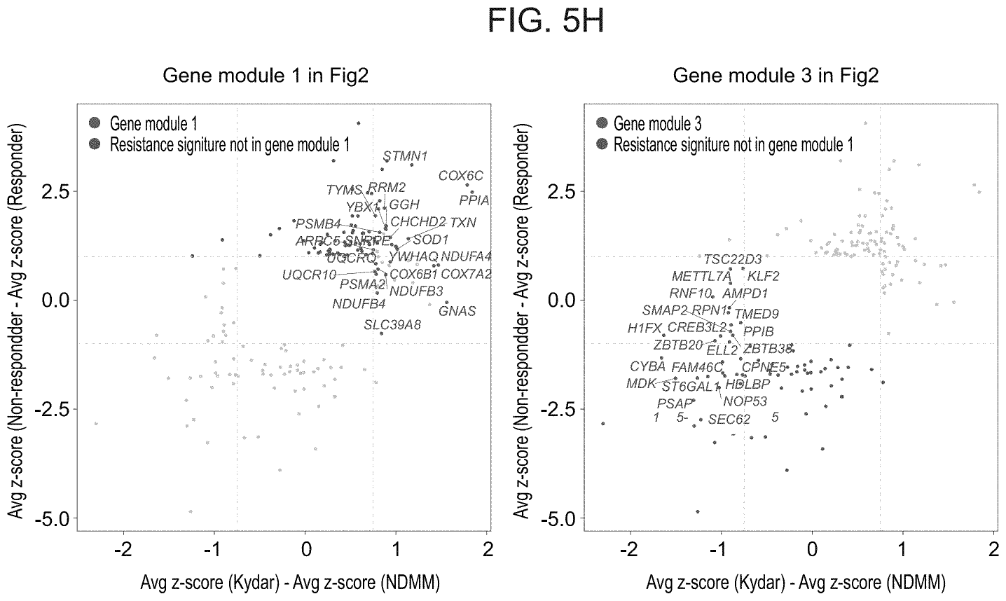

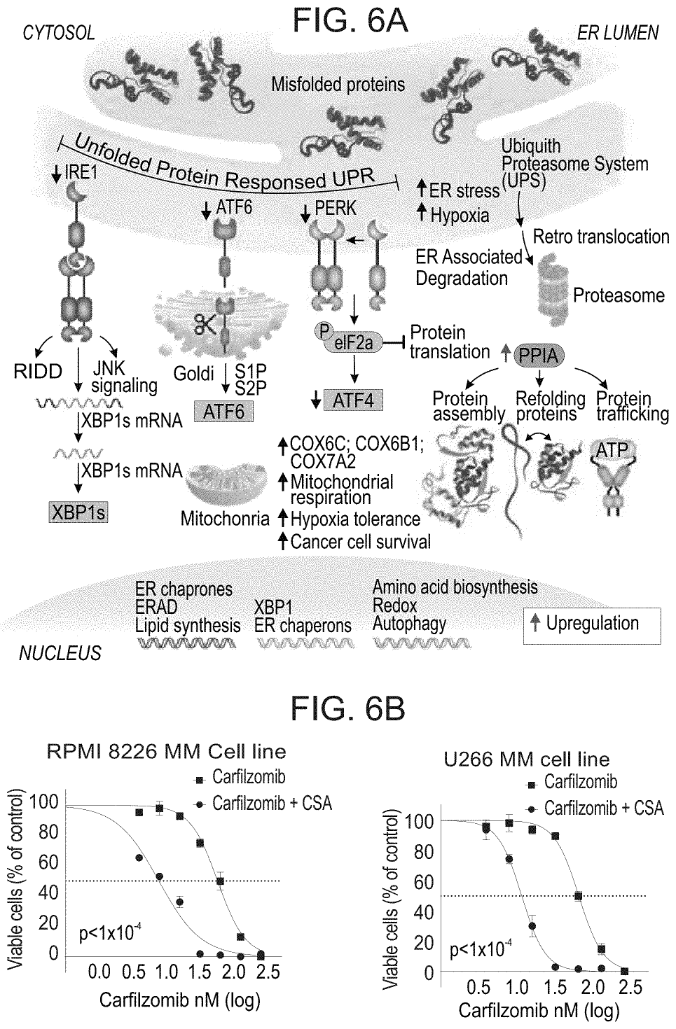

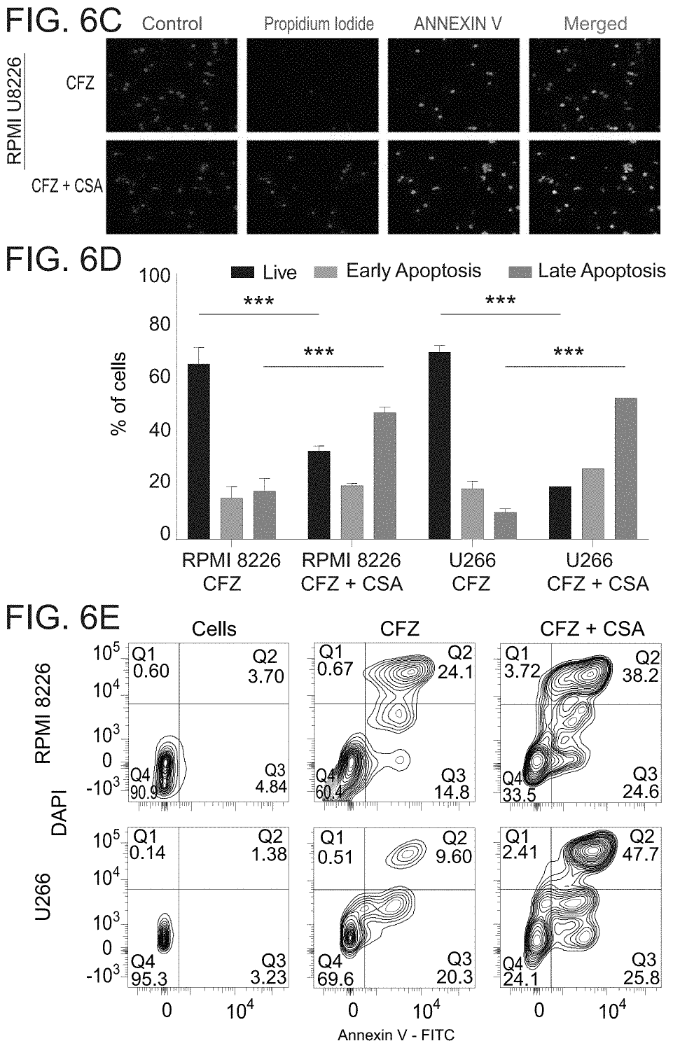

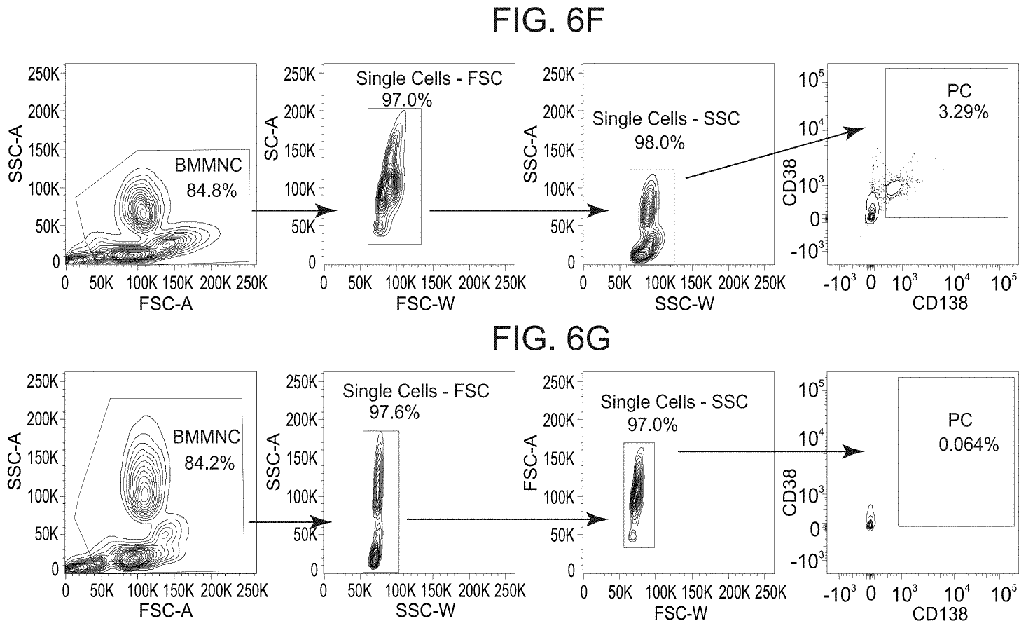

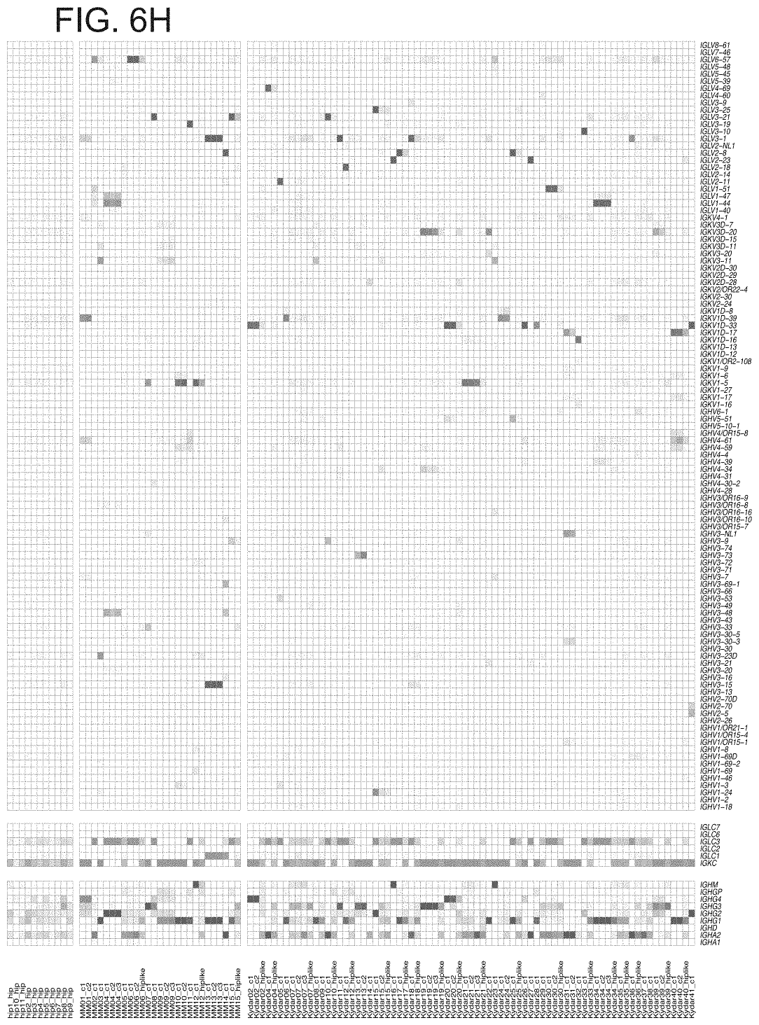

Since many of the genes associated with resistance (Table C/D) fall into pathways that potentially may synergize with current first line treatment, especially for patients with resistant signatures, the causality of one of some genes in the protein folding pathway, e.g., intracellular PPIA was determined. Using MM cell lines, and a PPIA small molecule inhibitor (Cyclosporine A), a robust and synergistic tumor killing is demonstrated, achieved by combining Carfilzomib together with Cyclosporine A (see FIGS. 6A-E).

Thus, according to an aspect of the invention, there is provided a method of treating a subject diagnosed with multiple myeloma (MM), the method comprising administering to the subject a therapeutically effective amount of a proteasome inhibitor and at least one agent which specifically down-regulates activity or expression of PPIA and/or RRM2, thereby treating the subject.

According to an additional aspect there is provided a combination comprising a therapeutically effective amount of a proteasome inhibitor and at least one agent which specifically down-regulates activity or expression of PPIA and/or RRM2 for use in treating multiple myeloma (MM) in a subject in need thereof.

According to an additional or alternative aspect, there is provided a method of treating a subject diagnosed with MM selected expressing intracellular PPIA and/or RRM2 above a predetermined threshold, the method comprising administering to the subject a therapeutically effective amount of at least one agent, which specifically down-regulates activity or expression of PPIA and/or RRM2, thereby treating the subject.

According to an additional or alternative aspect, there is provided at least one agent which specifically down-regulates activity or expression of PPIA and/or RRM2 for use in treating multiple myeloma (MM) selected expressing intracellular PPIA and/or RRM2 above a predetermined threshold in a subject in need thereof.

According to an additional or alternative aspect, a method of predicting responsiveness to treatment in a subject diagnosed with MM, the method comprising determining a level of intracellular PPIA and/or RRM2 in PC of the subject (first line naïve patient or a subject that has already been treated with an anti MM drug/treatment), wherein upregulation of said intracellular PPIA and/or RRM2 as compared to expression of same in normal PC is indicative of poor responsiveness to treatment with a proteasome inhibitor.

The agent is typically directed to PPIA or RRM2, hence when combined there is at least one agent targeting PPIA and another one targeting RRM2.

As used herein “RRM2” refers to Ribonucleotide Reductase Regulatory Subunit M2 gene. This gene encodes one of two non-identical subunits for ribonucleotide reductase. This reductase catalyzes the formation of deoxyribonucleotides from ribonucleotides. Synthesis of the encoded protein (M2) is regulated in a cell-cycle dependent fashion. Transcription from this gene can initiate from alternative promoters, which results in two isoforms that differ in the lengths of their N-termini. Exemplary Genbank Accession Numbers for human RNA include NM-01165931 and NM_001034. Likewise, Accession Numbers for the protein include NP_001025 and NP_001159403.

As used herein “PPIA” refers to peptidylprolyl isomerase A (PPIA), also known as cyclophilin A (CypA) or rotamase A is an enzyme that in humans is encoded by the PPIA gene on chromosome 7. As a member of the peptidyl-prolyl cis-trans isomerase (PPIase) family, this protein catalyzes the cis-trans isomerization of proline imidic peptide bonds, which allows it to regulate many biological processes, including intracellular signaling, transcription, inflammation, and apoptosis. Exemplary Genbank Accession Numbers for human RNA include NM_203431NM_001300981, NM_021130, NM_203430. Likewise, Accession Numbers for the protein include NP_001287910, NP_066953.

According to some embodiments, expression of intracellular PPIA above a predetermined threshold is above 1.2 unique RNA molecules (UMI) per cell on average as determined by scRNA sequencing of myeloma cells from bone marrow aspiration sample.

According to some embodiments, the inhibitor is such that down regulates activity or expression of the intracellular enzyme, e.g., catalytic activity inhibitor (i.e., trans isomerization of proline imidic peptide bonds in oligopeptides and acceleration of folding of proteins).

As used herein “an agent capable of down-regulating a target gene” refers to down-regulation of expression (mRNA or protein translation) or down-regulation of activity, e.g., catalytic activity, enzymatic function (e.g., using an antibody, peptide or a small molecule).

As used herein the phrase “downregulates expression” refers to downregulating the expression of a protein (e.g. the protein product of the target gene, e.g., PPIA OR RRM2) at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

For the same culture conditions the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating the target gene, e.g., PPIA, RRM2 expression are described in details hereinbelow.

As used herein “expressing intracellular PPIA and/or RRM2 above a predetermined threshold” refers to the level of expression (RNA or protein) in plasma cells (non-secreted, non-extracellular) as compared to control of plasma cells of a healthy subject i.e., not affected with multiple myeloma and having normal plasma cells.

“Above a predetermined threshold” means at least 10%, 20%, 30%, 40%, 50%, 60%, 70%, 80%, 90%, at least 2 fold, 3 fold, 4, fold, 5 fold, 10 fold, 50 fold, 100 fold higher compared to the aforementioned control.

Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding the target protein.

According to specific embodiments, the downregulating agent directly interacts with the target gene or expression product thereof.

According to specific embodiments, the agent directly binds the target gene of expression product thereof.

According to specific embodiments, the agent indirectly binds the target gene of expression product thereof (e.g. binds an effector of the target gene or expression product thereof).

According to specific embodiments the downregulating agent is an RNA silencing agent or a genome editing agent.

Thus, downregulation of the target gene or expression product thereof can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Nucleic acid agents can also operate at the DNA level as summarized infra.

Downregulation of the target gene can also be achieved by inactivating the gene via introducing targeted mutations involving loss-of function alterations (e.g. point mutations, deletions and insertions) in the gene structure.

As used herein, the phrase “loss-of-function alterations” refers to any mutation in the DNA sequence of a gene, which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the mutation may be in a homozygous form or in a heterozygous form.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

According to specific embodiments the agent capable of downregulating a target gene product is an antibody or antibody fragment capable of specifically binding the protein. Preferably, the antibody specifically binds at least one epitope of the target protein. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

As the target protein is localized intracellularly, an antibody or antibody fragment capable of specifically binding the target protein is typically an intracellular antibody.

Another agent which can be used along with some embodiments of the invention to downregulate the target protein is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another contemplated agent which can be used to downregulate any of the above described proteins includes a proteolysis-targeting chimaera (PROTAC). Such agents are heterobifunctional, comprising a ligand which binds to a ubiquitin ligase (such as E3 ubiquitin ligase) and a ligand to one of the above described proteins (e.g. PPIA or RRM2) and optionally a linker connecting the two ligands. Binding of the PROTAC to the target protein leads to the ubiquitination of an exposed lysine on the target protein, followed by ubiquitin proteasome system (UPS)-mediated protein degradation.

Another agent capable of downregulating the target protein would be any molecule which binds to and/or cleaves the target protein. Such molecules can be a small molecule, antagonists, or inhibitory peptide or dominant negative peptides.

Numerous inhibitors are known in the art for PPIA. U.S. Patent Application Nos. 20180023058, 20160289271, 20160039880, 20130336929 and 20130324480 disclose various cyclosporine analogs. Other nucleic acid silencing molecules, antibodies and genome editing agents for downregulating PPIA are commercially available such as from Horizon Ltd. and Abbexa Ltd.

According to a specific embodiment, the PPIA inhibitor is cyclosporine A (branded as e.g., Neoral™, Sandimmune™).

Numerous inhibitors are known in the art for RRM2. These include, but are not limited to cladribine, cytarabine (Cladribine, Gemcitabine, Hydroxyurea, Clofarabine, Gallium nitrate, Cytarabine, Fludarabine, Motexafin gadolinium, Triapine, GTI 2040, LOR 2040, Imexon), COH29, COH20.

Down-regulation of RRM2 at the level of RNA or DNA is disclosed in various scientific publications including Zheng et al. 2018 Molecular Therapy: Nucleic Acids Vol. 12:805; Xue et al. Int. J. Med. Sci. 2019; 16(11): 1510-1516; Heidel et al. Clin Cancer Res 2207 2007; 13(7) Apr. 1, 2007.

Numerous inhibitors of proteasome activity are known in the art some of which are already in clinical use. The first non-peptidic proteasome inhibitor discovered was the natural product lactacystin. Others include Disulfiram, Epigallocatechin-3-gallate, Marizomib (salinosporamide A) has started clinical trials for multiple myeloma, Oprozomib (ONX-0912), delanzomib (CEP-18770) have also started clinical trials, Epoxomicin is a naturally occurring selective inhibitor, Beta-hydroxy beta-methylbutyrate is a proteasome inhibitor in human skeletal muscle in vivo.

Approved medications include, Bortezomib (Velcade) was approved in 2003. This was the first proteasome inhibitor approved for use in the U.S. Its boron atom binds the catalytic site of the 26S proteasome, Carfilzomib (Kyprolis) was approved by the FDA for relapsed and refractory multiple myeloma in 2012. It irreversibly binds to and inhibits the chymotrypsin-like activity of the 20S proteasome. Ixazomib (Ninlaro) was approved by the FDA in 2015 for use in combination with lenalidomide and dexamethasone for the treatment of multiple myeloma. It is the first orally-available proteasome inhibitor.

Other types of inhibitors for proteasomal activity are also known in the art including, peptides silencing molecules and the like.

According to a specific embodiment, the proteasome inhibitor is Carfilzomib.

According to a specific embodiment, the proteasome inhibitor is Carfilzomib in combination with Cyclosporine A or cladribine, optionally with dexamethasone.

Contemplated patients that can be treated as described herein are provided hereinabove (under the definition “subject”).

According to a specific embodiment, the subject exhibits primary resistance to first line treatment, e.g., of less than 4 months following an anti MM treatment initiation.

According to a specific embodiment, the subject exhibits early relapse following an anti MM treatment, e.g., of less than 18 months.

According to a specific embodiment, the proteasome inhibitor and the at least one agent are in separate formulations (e.g., that can be sold as a kit or obtained from different manufacturers or a single manufacturer is separate packaging).

According to a specific embodiment, the proteasome inhibitor and the proteasome inhibitor and said at least one agent are in a single formulation. (also referred to as a pharmaceutical composition).

The agent/inhibitors of some embodiments of the invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent/inhibitor accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent/inhibitor (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (agent/inhibitor) effective to prevent, alleviate or ameliorate symptoms of a disorder (e.g., cancer. melanoma) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide agent/inhibitor levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Treatments as described herein may be combined with Gold standard treatment modalities against MM including but not limited to,

As used herein the term “about” refers to ±10%.

The terms “comprises”, “comprising”, “includes”, “including”, “having” and their conjugates mean “including but not limited to”.

The term “consisting of” means “including and limited to chemotherapy and/or (autologous) hematopoietic stem-cell transplantation (ASCT).

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

### Methods

Obtaining Patients Plasma Cells from Iliac Crest Aspirates

Patients suspected for plasma cell neoplasm and relapse refractory patients were recruited to the study from hematology departments in 13 medical centers in Israel. After informed consent (in accordance with Helsinki declaration), bone marrow aspiration was placed in EDTA-containing tubes (Beckton Dickenson), diluted 1:1 with ice cold FACS buffer (EDTA pH8.0 2 mM, BSA 0.5% in PBS), placed on ice and immediately transported to the lab.

Flow Cytometry Single Cell Sorting for Mars-Seq 2.0

Bone marrow cells were diluted 2:1 in ice cold FACS buffer (EDTA pH8.0 2 mM, BSA 0.5% in PBS), washed and strained with a 100 μm strainer. Mononuclear cell separation was performed by density centrifugation media (Ficol-paque, GE Life Sciences) in a 1:1 ratio with marrow cells. Centrifugation (460 g, 25 min) was performed at 10° C., and the mononuclear cells were carefully aspirated and washed with ice cold FACS buffer. After red blood cell lysis (Sigma) for 5 min at 4° C. and washing, bone marrow cells from relapse refractory patients were stained with prior magnetic CD138⁺ beads enrichment (Miltenyi). Cells were washed and stained with antibodies (all from Cytognos or BD Biosciences): CD38, CD138, CD56, CD19, CD117, CD27, CD45, CD81. Samples were filtered through a 40 m strainer before commencing sorting. Single cell sorting was performed using either FACS SORP-ArialI or AriaFusion (BD Biosciences, San Jose, Calif.). After doublets exclusion, isolated cells were single-cell index-sorted into 384-well cell capture plates containing 2 L of lysis solution and barcoded poly(T) reverse-transcription (RT) primers for single-cell RNA-seq. Four empty wells were kept in each 384-well plate as a no-cell control for data analysis. Immediately after sorting, each plate was spun down to ensure cell immersion into the lysis solution, snap frozen on dry ice, and stored at −80° C. until processed. Cells were analyzed using BD FACSDIVA software (BD Bioscience) and FlowJo software (FlowJo, LLC).

Massively Parallel Single-Cell RNA-Seq Library Preparation (MARS-Seq)

Single-cell libraries were prepared as previously described^{45,46,47}. Briefly, mRNA from cells sorted into barcoded cell capture plates and converted into cDNA, later pooled by using an automated pipeline. The pooled sample is then linearly amplified by T7 in vitro transcription, and the resulting RNA is fragmented and converted into a sequencing-ready library by tagging the samples with pool barcodes and Illumina adapters during ligation, RT, and PCR. Each pool of cells was tested for library quality and concentration was assessed as described earlier^{45,46,47}. Overall, barcoding was done in three levels: Cell barcodes allow attribution of each sequence read to its cell of origin, thus enabling pooling; Unique Molecular Identifiers (UMIs) allow tagging each original molecule in order to avoid amplification bias; and plate barcodes allow elimination of the batch effect.

Analysis of Single-Cell RNA-Seq Data

MARS-seq libraries, pooled at equimolar concentrations, were sequenced using an Illumina NextSeq 500 sequencer, at a sequencing depth of 20K-50K reads per cell. Reads are condensed into original molecules by counting same unique molecular identifiers (UMI). The present inventors used statistics on empty-well spurious UMI detection to ensure that the batches the present inventors used for analysis showed a low level of cross single-cell contamination (less than 3%). MARS-seq reads were processed as previously described⁴⁷. Reads were mapped to human reference genome hg38 using HISAT (version 0.1.6); reads with multiple mapping positions were excluded. Reads were associated with genes if they were mapped to an exon, using the UCSC genome browser for reference. Exons of different genes that shared genomic position on the same strand were considered a single gene with a concatenated gene symbol.

Metacell Modeling for MARS-Seq Data

To analyze the MARS-seq data from all the samples, the present inventors used the MetaCell package²⁶ with the following specific parameters. The present inventors removed specific mitochondrial genes and immunoglobulin genes. The present inventors then filtered cells with less than 300 UMIs or total fraction of mitochondrial gene expression exceeding 50%. Gene features with high variance to mean were selected using the parameter Tᵥₘ>0.2 and minimal total UMI >100. From the gene features, the present inventors excluded high abundance lincRNA and genes linked with poorly supported transcriptional models (such as genes annotated with the prefix “AC[0-9]”, “AL[0-9]”, etc.). Annotation of the metacell model was done using the metacell confusion matrix and analysis of marker genes. The present inventors identified non-PC cells using straightforward analysis of known cell type markers (e.g., COL1A2—fibroblast cells, TRAC-T cells, CIQA—macrophages, S100A8—monocytes, and more), and cross-validation with the existing data from previous work²². Non-PC cells, such as fibroblast cells, monocytes, macrophages, etc., were considered as contamination, and were removed before performing the final clustering. In the final clustering, the gene feature selection strategy described above retained a total of 2,038 genes for the computation of the Metacell balanced similarity graph. The present inventors used K=500 bootstrap iterations. Metacell splitting was performed by clustering the cells within each metacell and splitting it, if distinct clusters are detected.

Z-Score of the Gene Expression in Malignant PC from NDMM and PRMM Patients

In order to provide robust estimates of malignant PC gene expression, the present inventors calculated a z-score for each gene of each malignant PC clone from individual NDMM and PRMM patients according to the following steps. First, for a given malignant PC clone, the present inventors randomly selected 100 cells from the clone and 100 cells from healthy control group, and the present inventors assured that the present inventors always selected relatively the same number of healthy PC from each healthy control donor. Second, the present inventors computed a p-value using Mann-Whitney test for each gene comparing the normalized UMI counts in the 100 malignant PC and in the 100 healthy PC. This p-value then was converted into a z-score. For each malignant PC clone, the present inventors performed the previous two steps 100 times to obtain 100 z-scores for each gene, and the present inventors calculated the average value of the 100 z-scores to represent the relative expression of each gene in the given malignant PC clone compared with the healthy PC. The present inventors performed the same calculation for all the malignant PC clones.

Detecting Differential Genes Between Malignant PC from Two Different Patient Groups

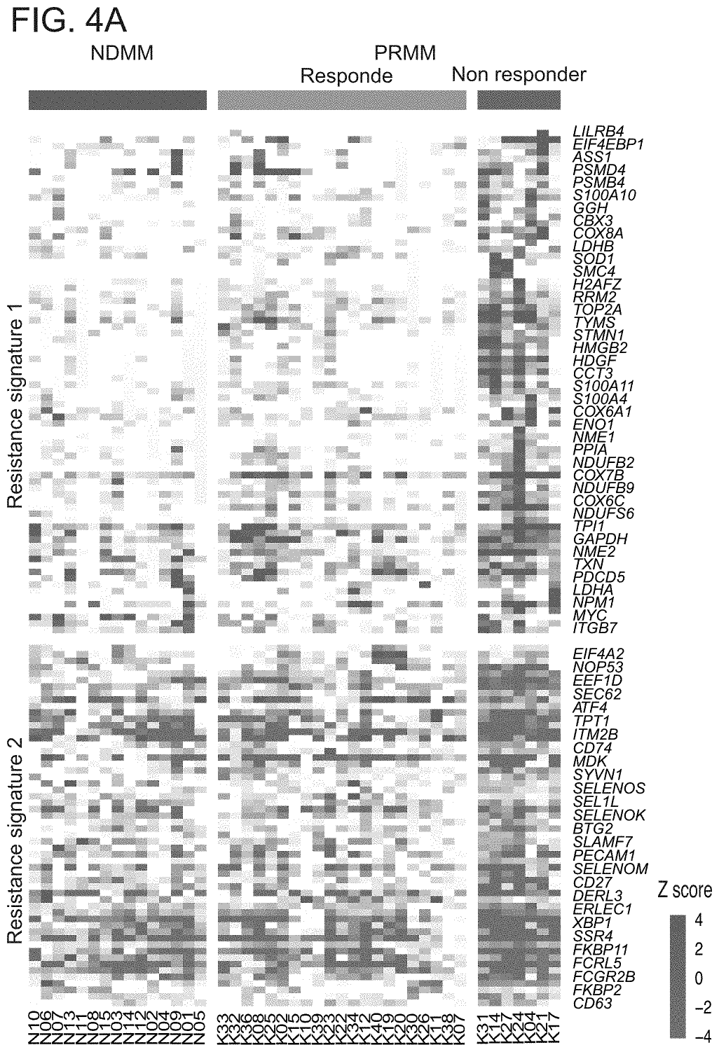

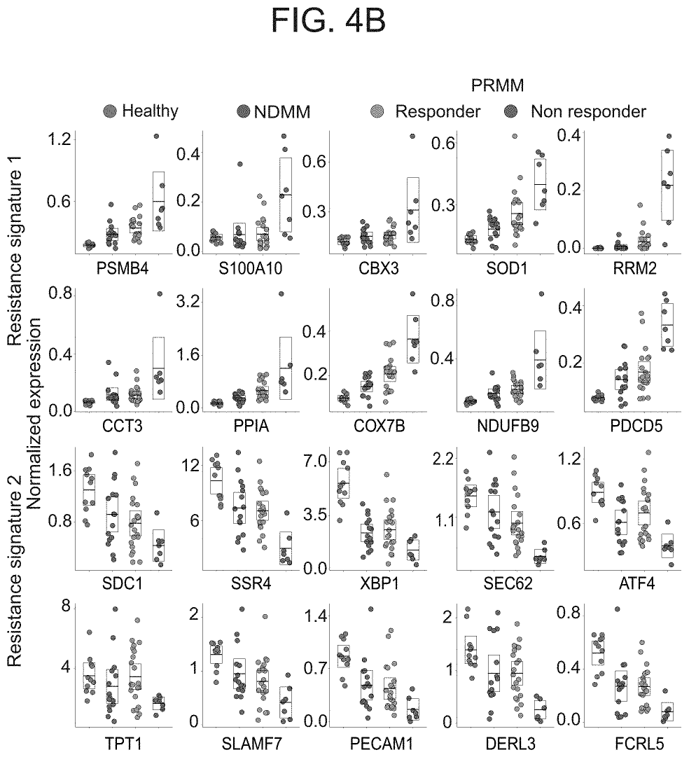

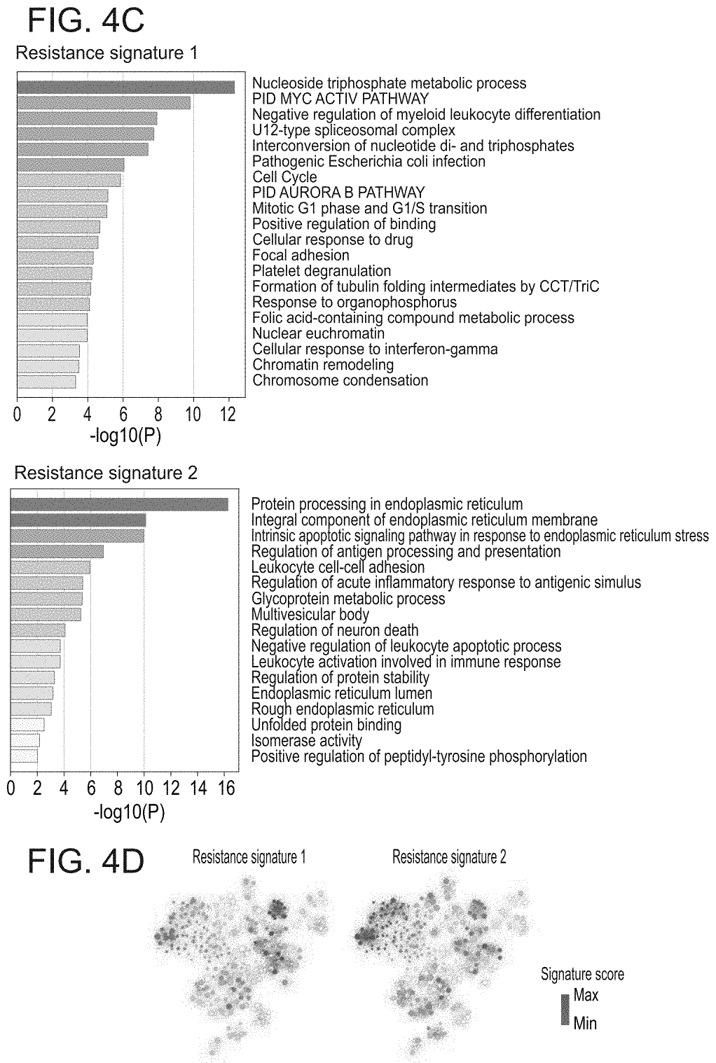

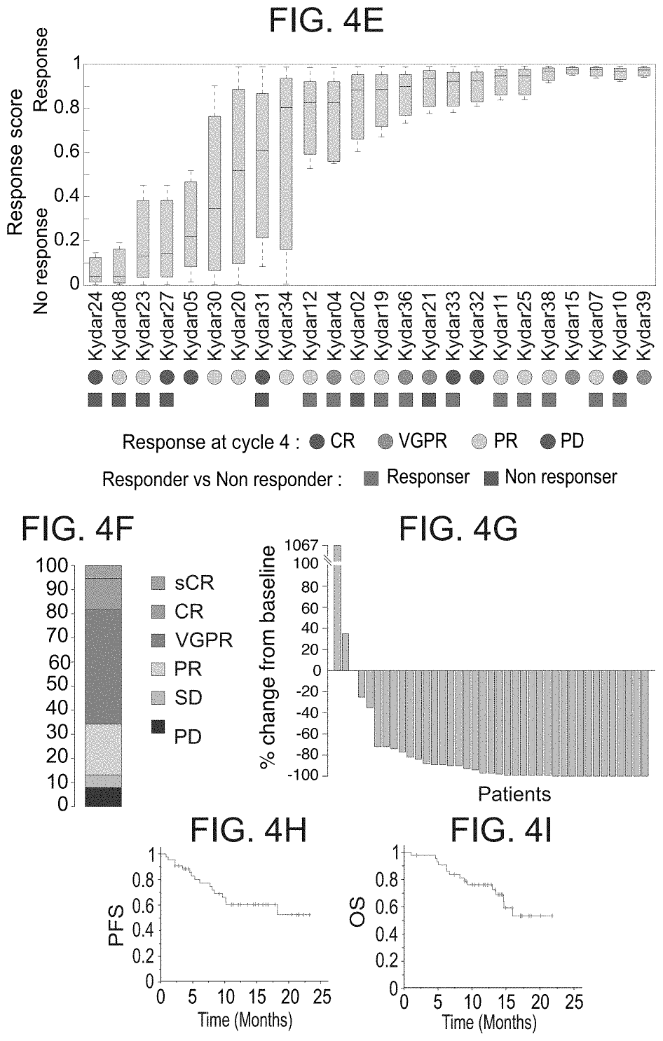

In order to detect the differential genes between malignant PC from NDMM and PRMM patients, the present inventors compared the z-scores of each gene from the NDMM and PRMM patients. First, for each gene, the present inventors calculated a t-statistic Tᵣₑₐₗ between the expression z-scores of NDMM patients and the expression z-scores of PRMM patients. Second, the present inventors shuffled the labels of the z-scores of the gene and calculated a t-statistic T_{bg} between the expression z-scores of the shuffled NDMM and PRMM patient groups. The present inventors performed the shuffling 10 k times. Therefore, the present inventors can compute an empirical p value for each gene by calculating the frequency of Tᵣₑₐₗ>T_{bg} or Tᵣₑₐₗ<T_{bg}. The present inventors used the same computation method to detect the differential genes between malignant PC from PRMM responder and non-responder groups in FIG. 4A-I.

Cross-Referencing with Known Myeloma Datasets (MMRF—CoMMpass)

We calculated module 1 gene score for each patient from the Multiple Myeloma Research Foundation's CoMMpass Interim Analysis 14 (n=908 patients with clinical and RNA-seq data) using the Salmon v7.2 filtered gene transcripts per million (TPM) normalized gene expression table. The score was defined as the average log transformed TPM of the 25 genes comprising module 1. For Kaplan-Meir analysis the present inventors defined patients with module 1 score >200 as high module 1.

Shallow Neural Network Model to Predict Treatment Response

To predict response to treatment the present inventors train a shallow neural network with 10 hidden layers using Matlab R2018A deep learning toolbox. For each patient the present inventors trained a separate model on the entire cohort excluding the target patient (leave-one-out strategy). The model was trained on the integer single cell matrix down sampled to 500 UMIs per cell using 2,038 variable gene as features. The present inventors used ‘trainscg’ as the network training function that updates weight and bias values according to the scaled conjugate gradient method with default values. The network performance was calculated using the ‘crossentropy’ function with 90/10 training/validation ratio. The final score is obtained for each cell by applying the network on the target patient's cells and displayed as box plot showing the median value and 0.25, 0.75 quantiles.

Gene Module Score at Single Cell Level and Metacell Level

Given any list of gene module, the present inventors defined the module scores for each single cell by averaging the log 2 (UMI×7+1), and the present inventors defined the module scores for each metacell by averaging the metacell log enrichment scores (lfp values) of the genes in the set. Note that using this approach the present inventors limited the contribution of highly expressed genes to the score. At single cell, the calculation overcome the sparseness of experimental detection. At metacell level, the present inventors relied on the regularization of the metacell computation of gene enrichment scores to restrict the noise levels inflicted over the gene module scores. The present inventors used the same computation method to calculate the resistance signature score in FIG. 4A-I.

Myeloma Cell Lines and Drugs

RPMI-8226 and U266 myeloma cell lines were purchased from American Type Culture Collection (Manassas, Va.). Cells were cultured using an aseptic technique in RPMI medium (Gibco) supplemented with 10% heat-inactivated fetal bovine serum, 1 mM sodium pyruvate, 2 mM L-glutamine, 1% penicillin-streptomycin (ThermoFisher Scientific). Cells were stored in 10-50 ml flasks (Corning) in an incubator (ThermoFisher Scientific) with humidified air and 5% CO2, at 37° C. at a concentration of 0.5-1 million cells per ml. Cell lines were validated for lack of mycoplasma infection using primers for mycoplasma-specific 16S rRNA gene region (EZ-PCR Mycoplasma Kit, Biological Industries, Beit Ha'emek, Israel). Carfilzomib and Cyclosporine A (CsA) were purchased from LC Laboratories, Woburn, Mass., USA.

Cell Proliferation Assays

Myeloma cell lines were seeded in triplicates in 96-well round-bottom microplates at a density of 5×10⁴ cells/well and incubated with or without drugs for 48 h at 37° C. After incubation, MTS terazolium compound (CellTiter 96 AQueous One Solution Cell Proliferation Assay; Promega, Madison, Wis., USA) was added and the cells were incubated for 2-4 h. The absorbance was measured at a wavelength of 490 nm using a microplate reader (Synergy H1 microplate reader BioTek, Winooski, Vt., USA) and expressed as a percentage of the value of the corresponding untreated cells.

Dose Response Assays

MTS assays were used as previously described to establish dose-response curves and to determine the half maximal inhibitory concentrations (IC50) of single-dose Carfilzomib or Cyclosporine A and the combination for each cell line. Carfilzomib and CSA were administered at plating with a serial dilution starting at doses of 1 M and 100 g, in assay buffer, respectively. Cells were harvested at 48 hours to determine half maximal inhibitory concentrations (IC50) of each single drug. CsA IC50 of both cell lines (3 g) was used at fixed concentration to determine synergism with Carfilzomib.

IC50 Analysis

IC50 values were derived by a dose-response inhibition (variable slope) curve and were fitted using non-linear regression using GraphPad Prism software. The reported data are average of at least three independent experiments. Extra sum-of-squares F test was used to test whether IC50 values differed between groups using GraphPad Prism (GraphPad Software, Inc., La Jolla, Calif., USA).

Immunofluorescence Analysis of Apoptosis and Cell Death

Myeloma cell lines were seeded and incubated at 96 well plates as previously described, with different treatments: Cells only, Carfilzomib only (IC50) and Combined treatment (CsA IC50+Carfilzomib IC50). Cells were then harvested and centrifuged for 5 min at 300 g, 4° C. Cells were washed twice with cold Biolegend cell staining buffer (Biolegend, 420201) and then were resuspended in 50 μl of Annexin V binding buffer (Biolegend, 422201) and were stained with 1 μl FITC Annexin V. Cells were gently vortexed and incubated for 15 min at RT (25° C.) in the dark. 21 of Propidium iodide solution (Sigma-Aldrich, P4864, 1:50) was added at the last 5 min of staining. 35 μl of 16% Formaldehyde solution (Thermo Scientific, 28906) were directly added and was gently mixed using a pipette, then cells were incubated for 10 minutes at RT (25° C.) in the dark. Cells were centrifuged for 5 min at 300 g, 4° C. and were resuspended with 100 μl of cold Biolegend cell staining buffer, and 1.241 of 10% PBST (Triton X-100, Sigma-Aldrich, T8787) and were gently mixed and incubated for 5 min at RT (25° C.) in the dark. For detection of cell nuclei, DAPI was added for 2 min. After spin down, all the cells were applied to slides, mounted with SlowFade Diamond Antifade Mountant (Thermo Scientific, S36963), sealed with coverslips. Microscopic analysis was performed using a laser-scanning confocal microscope (Zeiss, LSM880). Images were acquired and processed by Imaris software; all the thresholds are fixed through the whole experiment (Bitplane).

FACS Analysis of Apoptosis and Cell Death

Myeloma cell lines were seeded and incubated at 96 well plates with different treatments as previously described. Cells were then harvested and centrifuged for 5 min at 300 g, 4° C. Cells were washed twice with cold Biolegend cell staining buffer (Biolegend, 420201) and then were resuspended in 50 μl of Annexin V binding buffer (Biolegend, 422201) and were stained with 1 μl FITC Annexin V. Cells were gently vortexed and incubated for 15 min at RT (25° C.) in the dark. 241 of Propidium iodide solution (Sigma-Aldrich, P4864, 1:50) was added at the last 5 min of staining. Cells were centrifuged for 5 min at 300 g, 4° C. and resuspended with 50 μl of Annexin V binding buffer. Cells were analyzed for proliferation using LSRII FACS analyzer (BD) and FlowJo software (FlowJo, LLC).

Statistical Analysis

Statistical analysis Data was presented as mean (±SEM) of three independent experiments. Comparisons between two groups of samples were evaluated using the t-test or two-way ANOVA. All P-values reported were two tailed and statistical significance was defined as P less than 0.05. All statistical analyses were conducted using R software (R Foundation for Statistical Computing, Vienna, Austria) or GraphPad Prism (GraphPad Software, Inc., La Jolla, Calif., USA).

KYDAR Clinical Trial

Eligibility Criteria

Patients were ≥18 years of age and had documented myeloma as per IMWG criteria⁴⁸ and started a bortezomib-based induction with corticosteroids, with or without alkylators, with or without IMiDs (thalidomide lenalidomide or pomalidomide), within 24 months prior to study enrollment had and not received any 2^{nd} line treatment. Patients have failed to achieve a minimal response (MR) after 2 cycles or a partial response (PR), after 4 cycles of a bortezomib-containing therapy [cohort A], or progressed within 18 months from induction treatment start (response defined by international Myeloma Working Group [IMWG] criteria) [cohort B], had measurable disease at time of enrollment as per IMWG criteria. Patients were not planned for auto-transplant: either determined by investigator to be transplant-ineligible, or post ASCT and progressed within 18 months from induction treatment start or fail to achieve at least PR post ASCT. Patients were required to have hemoglobin ≥8 g/dL, absolute neutrophil count ≥1.5 10⁹/L, platelet count ≥50,000/mm3 (or 30,000/mm2 if myeloma bone marrow involvement >50%); aspartate aminotransferase and alanine aminotransferase ≤3.0 times the upper limit of normal, calculated or measured creatinine clearance ≥20 mL/min, and left ventricular ejection fraction (LVEF) ≥40%. Patients were excluded if they had a diagnosis of monoclonal gammopathy of undetermined significance, smoldering MM, amyloidosis, or Waldenstrom disease. The study excluded patients with chronic obstructive pulmonary disease (with a forced expiratory volume in 1 second, 50% of predicted normal); moderate, severe, or uncontrolled asthma; or significant heart disease or active infection.

Study Design

This is an open-label non-randomized single arm multicenter prospective clinical and translational trial, in patients with multiple myeloma who received a bortezomib-based induction and either failed to achieve a timely response (<4 months) or progressed early (<18 months) after therapy initiation. Patients are treated with a quadruple regimen comprised of: Daratumumab 16 mg/Kg weekly during cycles 1-2, q14 days during cycles 3-6, thereafter monthly (1st dose cycle 1 may be split over 2 days); Once-weekly intravenous (IV) Carfilzomib on days 1, 8, 15, of cycle numbers 1-9 and Days 1 and 15 only of cycle numbers 10-18, at a dose of 20 mg/m² on day 1 of cycle 1; at dose of 56 mg/m² on all subsequent once weekly dosing days, alongside concomitant treatment with twice-weekly IV or oral dexamethasone 20 mg administered on Days 1-2, 8-9, 15-16, and 22-23 of a 28-day cycle, for cycles 1-2 followed by weekly 20 mg dexamethasone on subsequent cycles; and oral Lenalidomide 25 mg, administered on days 1-21 of a 28-day cycle (D-KRd). Frail patients (as per IMWG recommendations;⁵) receive Lenalidomide dose adjustment to 15 mg, and dexamethasone at 10 mg×2/week cycles 1-2 followed by 10 mg/week for subsequent cycles. The quadruple regimen is administered for 18 cycles, followed by long-term follow-up in which patients will receive standard of care treatment (lenalidomide maintenance or lenalidomide daratumumab). Pre-infusion medications included diphenhydramine (or equivalent), acetaminophen, and montelukast.

Study Endpoints and Analyses

The primary endpoints are the safety and tolerability of D-KRd. Safety evaluations included adverse event (AE) monitoring, physical examinations, electrocardiogram monitoring, clinical laboratory tests, vital sign measurements, and ECOG performance status. Toxicities are graded using the National Cancer Institute Common Terminology Criteria for Adverse Events Version 4 (CTCAE).

Secondary endpoints include overall response rate (ORR), progression free survival (PFS) and overall survival (OS). Response to treatment and disease progression are evaluated according to the IMWG response criteria at the end of each odd-numbered treatment cycle. M-protein measurements in serum and urine are assessed by each center's local laboratory. Serum and urine immunofixation electrophoresis (IFE) are performed at screening and when complete response (CR) is suspected. A daratumumab-specific IFE assay is used to confirm CR for patient samples in which daratumumab interference with IFE is suspected.

Single-cell transcriptional analysis is performed on bone marrow-aspirate samples obtained at study enrollment, 3, and 9 months, and to confirm CR or PD. Fresh samples are delivered at 4° C. within 90 minutes for plasma cell separation and single cell RNA analysis.

Statistical Analyses

A sample size of 40 was proposed to provide sufficient safety and preliminary efficacy data, enable detection of transcriptional patterns associated with therapy resistance and response. Descriptive statistics for treatment-emergent AEs (TEAEs) were summarized, including AEs of clinical interest: IRRs, infections, and cardiac function. Responses were categorized per IMWG criteria.

Progression free survival (PFS) and overall survival (OS), were documented. Continuous variables were described as the median and range of observations. Categorical data were described with contingency tables including frequency and percent. Median length of follow-up was observed using reverse censoring method. The median survival time and the probabilities of OS and PFS were estimated using the Kaplan-Meier method with the use of a stratified log-rank test. Hazard ratios and corresponding 95% confidence intervals were estimated with the use of a stratified Cox regression model. A two-sided P value of <0.05 was considered as statistically significant. SPSS software (IBM SPSS Statistics for Windows, version 25, IBM corp., Armonk, N.Y., USA, 2017) was used for all statistical analyses.

Study Oversight

The study was registered at ClinicalTrials.gov (#NCT04065789). The clinical study sites' institutional review boards or ethics committees approved this study. All patients provided written informed consent. The study design and analyses were reviewed by the investigators. The investigators and their research teams collected the study data. TASMC clinical unit together with the Amit laboratory team at Weizmann institute performed the final data analysis and verified the accuracy of the data. The study was overlooked by an independent data safety and monitoring board.

### Example 1

### NDMM and PRMM Patients Display Similar Signatures that Converge into Pre-Defined Malignant Drivers

In order to better understand early resistance mechanisms of PRMM patients an open-label single arm prospective clinical and translational trial combined with comprehensive scRNA-seq were designed. Patients with MM who received a bortezomib-based induction and either failed to achieve a timely response (<4 months/cohort A) or progressed early (<18 months/cohort B) were recruited. The designed therapeutic treatment includes a quadruple regimen comprised of Daratumumab, Carfilzomib, Lenalidomide and Dexamethasone (DARA-KRD). Detailed patient characteristics are detailed in Table 1.

TABLE 1
Patient characteristics

| Parameter | N (%) |
|---|---|
| Age, years (median, range) | 70 (40-85) |
| Male:Female | 23 (56):18 (44) |
| Myeloma type: | |
| IgG | 18 (44) |
| IgA | 6 (15) |
| Light chain only | 17 (41) |
| ISS | |
| I | 6 (23) |
| II | 9 (35) |
| III | 11 (42) |
| R-ISS | |
| I | 5 (21) |
| II | 9 (38) |
| III | 10 (42) |
| Fish Cytogenetics | |
| t(4:14) | 3 (8) |
| Del17p | 14 (39) |
| t(14:16) | 3 (8) |
| t(14:20) | 1 (3) |
| +1q21 | 16 (44) |
| −1p | 3 (8) |
| t(11:14) | 11 (31) |
| No adverse | 6 (17) |
| Any intermediate/high risk | 24 (67) |
| FISH | |
| Any high risk FISH | 16 (44) |
| Double hit myeloma | 11 (27) |
| Primary refractory (cohort A) | 20 (49) |
| Early relapse (cohort B) | 21 (51) |
| EMD at diagnosis | 18 (44) |
| Bone-adjacent | 9 (22) |
| Non-bone | 9 (22) |
| Time since myeloma | 8 (3-17.9) |
| diagnosis months (median, | |
| range) | |
| Frailty | |
| Fit | 18 (44) |
| Intermediate fit | 9 (22) |
| Frail | 14 (34) |
| Induction regimen | |
| VCD | 21 (51%) |
| VTD | 9 (22%) |
| VRD | 9 (22%) |
| Post ASCT | 16 (39%) |

ISS—international staging system;
R-ISS—revised-ISS;
EMD—extramedullary disease;
FISH—Fluorescence in situ hybridization;
Intermediate/high risk: t(4:14), Del17p, t(14:16), t(14:20), +1q21;
double hit: >1 intermediate/high risk aberration;
VCD—bortezomib/cyclophosphamide/dexamethasone;
VTD—bortezomib/thalidomide/dexamethasone;
VRD—bortezomib/lenalidomide/dexamethasone;
ASCT—autologous stem cell transplant

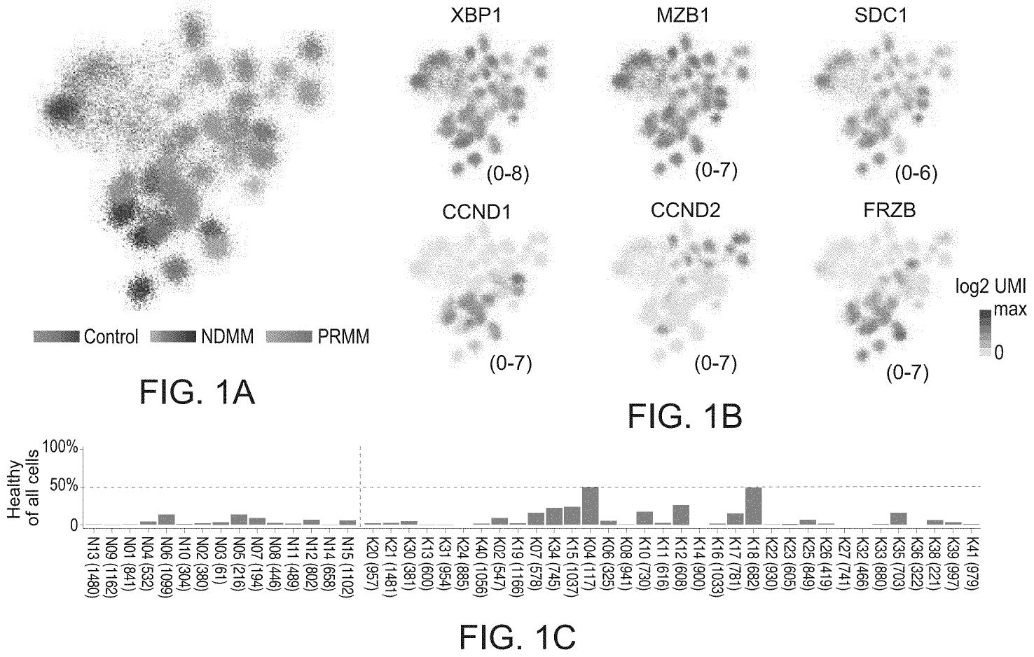

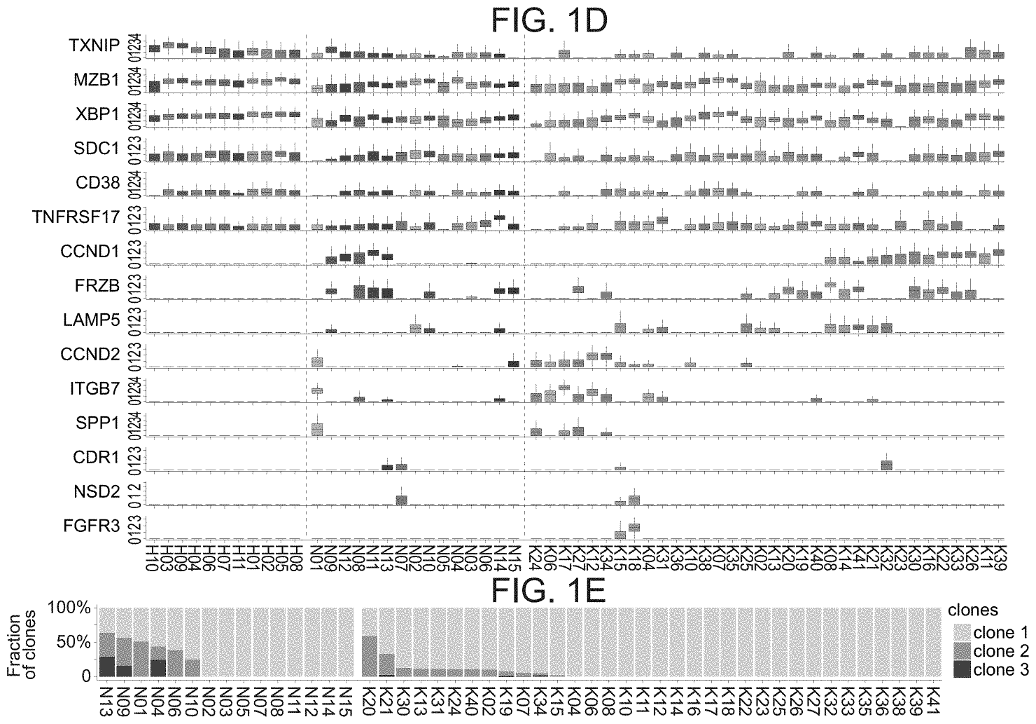

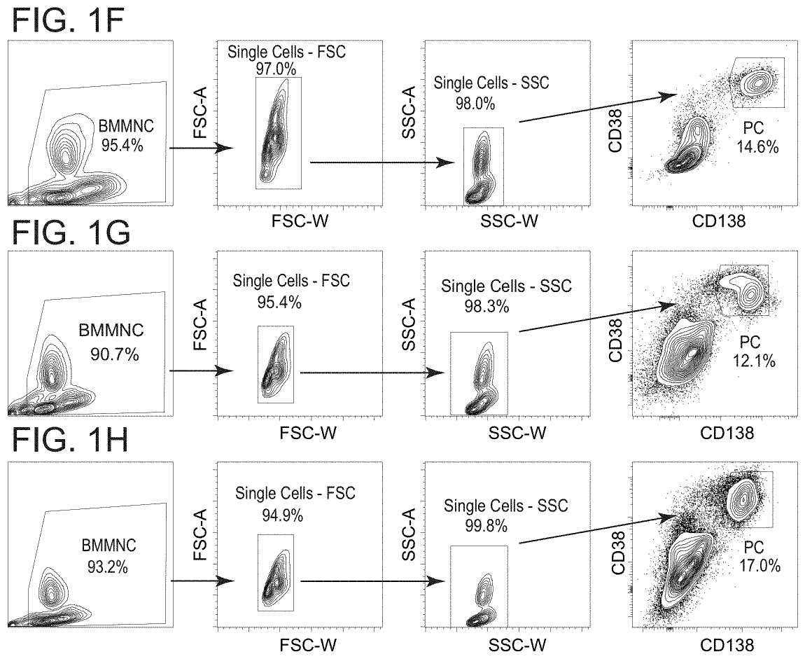

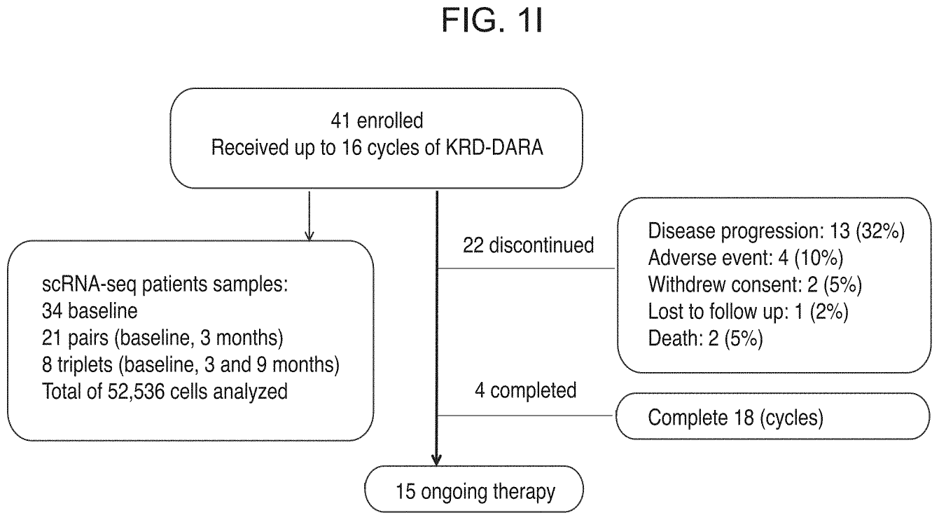

In order to characterize the full spectrum of healthy and malignant plasma cell states and pathways of these patients before treatment and longitudinally along the trial (baseline, cycle 4 and cycle 10), a robust protocol was designed for massively parallel single cell RNA-seq characterization of the BM PC using a validated antibody panel²² (CD45, CD19, CD56, CD81, CD27, CD117, CD38, CD138) (FIGS. 1F-H). 41 patients enrolled in the study. At time of data cutoff (Apr. 20, 2020), 15 patients are ongoing treatment, 4 have completed 18 cycles (10%), and 22 discontinued treatment, due to: disease progression 13 (32%), adverse events 4 (10%), withdrew consent 2 (5%), lost to follow-up 1 (2%) or death 2 (5%) (FIG. 1I).

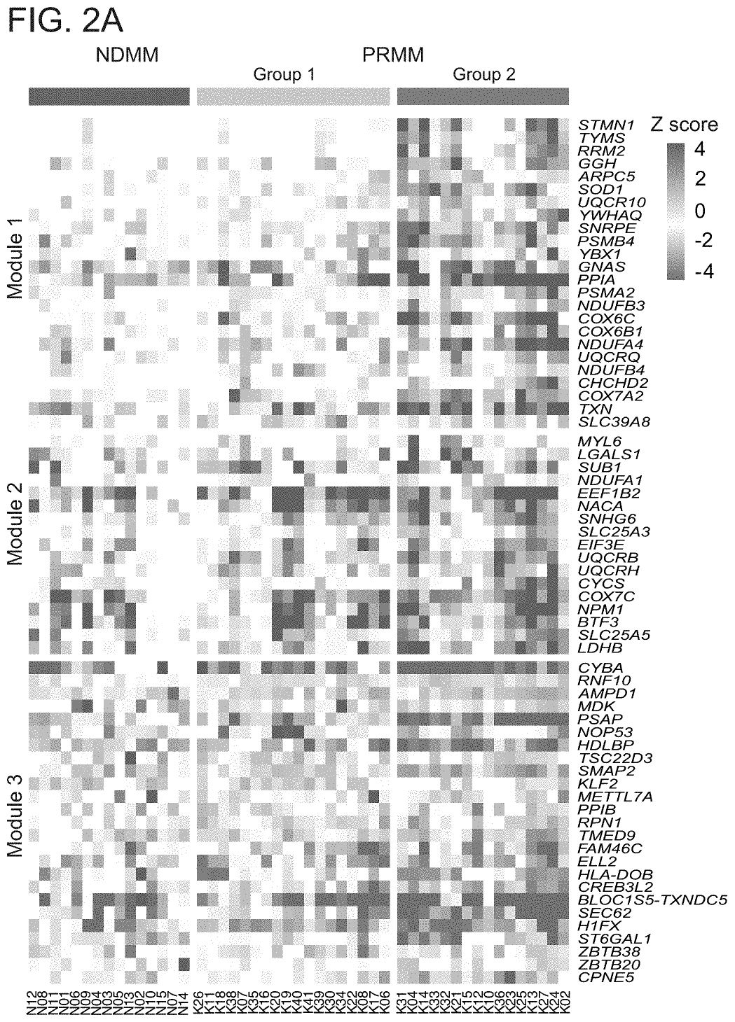

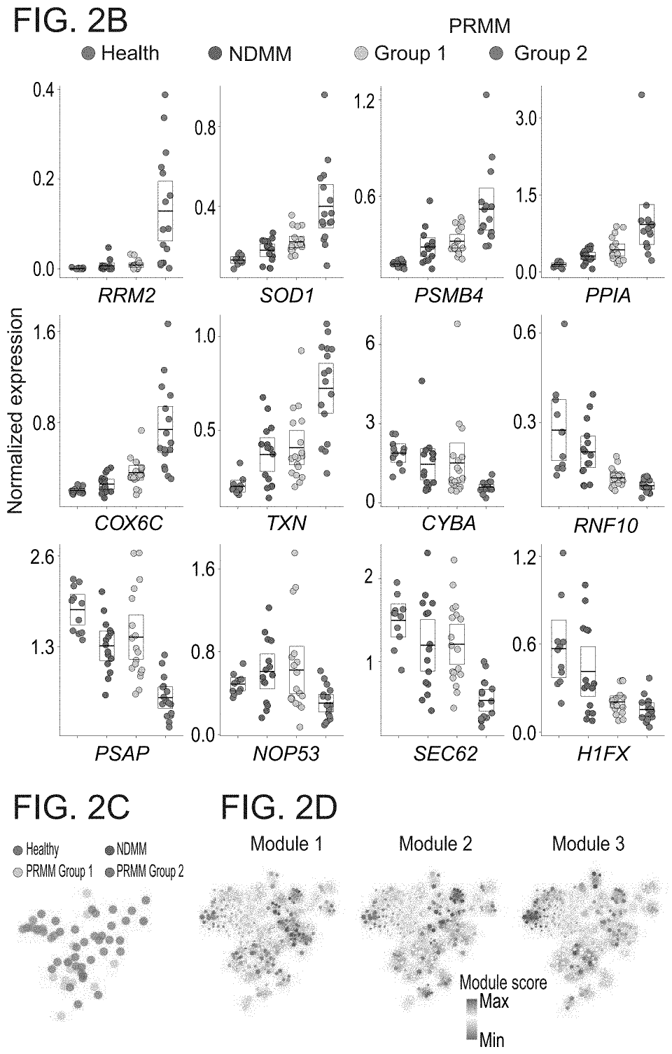

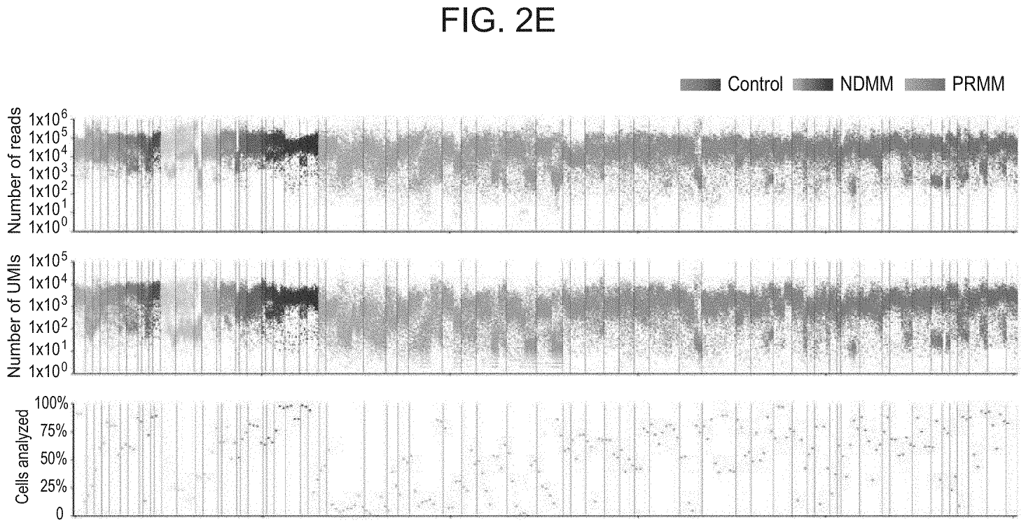

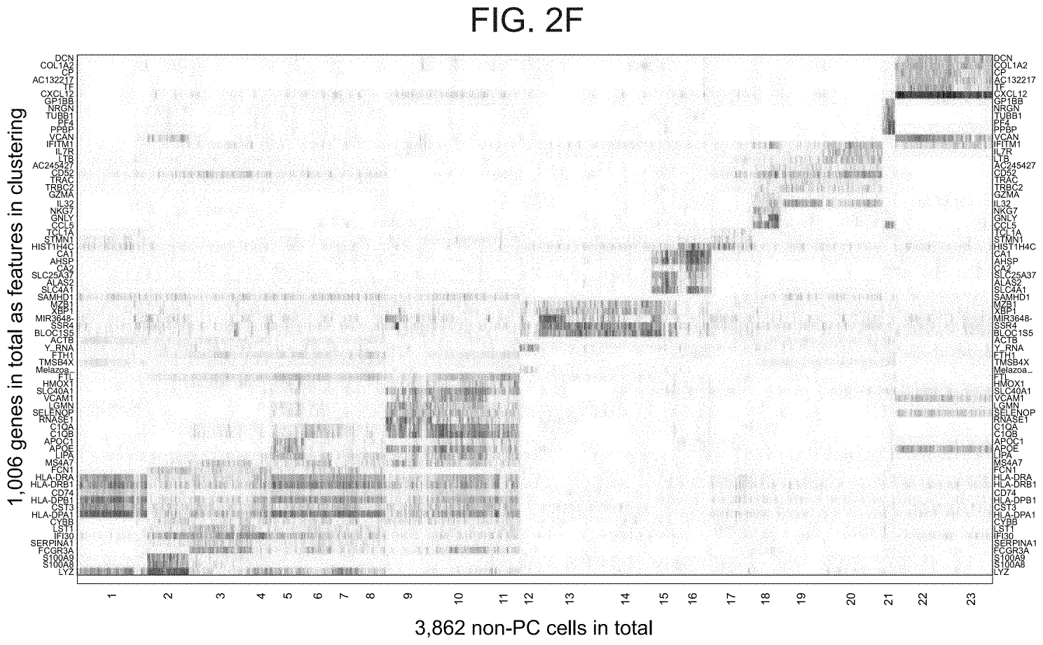

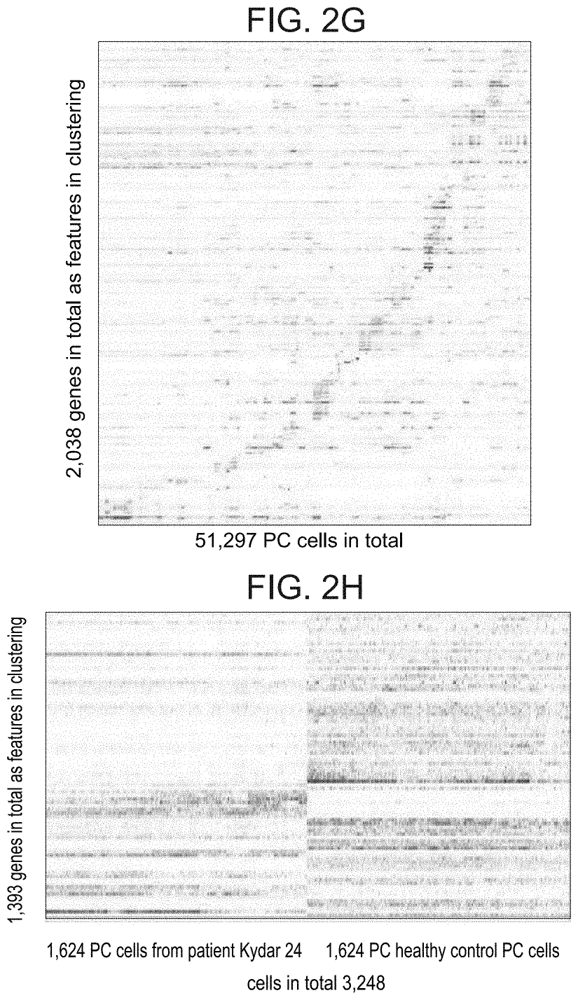

Bone marrow PC were sequenced (MARS-SEQ) from 34 baseline, 27 cycle-4 (3 months), and 10 cycle-10 (9 months) patients, in addition MARS-seq of BM PC were also analyzed from 15 newly diagnosed MM (NDMM) patients and 11 healthy aged control group undergoing hip replacement surgery as previously described²². Data was collected on a total of 95,380 cells, covering 51,297 QC-positive PC, following removal of contamination and low-quality cells (FIGS. 2E-F). The MetaCell algorithm²⁶ was used to identify homogeneous and robust groups of cells (“Metacells”; Methods) from scRNA-seq data, resulting in a detailed map of 260 metacells comprising the transcriptional subpopulations covering the spectrum of PC diversity: healthy PC, NDMM, and PRMM pre- and post-treatment (FIG. 1A). PC subpopulations were based on cluster-specific expression patterns of the 2,038 most variable genes discarding immunoglobulin (Ig) genes (FIG. 2G). Each patient was characterized by a unique PC transcriptional program while key plasma cell genes XBP1, MZB1, SDC1 (CD138), and TNFRSF17 (BCMA) are expressed in variable intensity among patients of all cohorts, including healthy controls (FIG. 1B, FIG. 2G). Although every patient displayed a unique transcriptional state, common overexpressed driver genes were detected which are shared across sub-groups of patients, such as CCND1, CCND2, and FRZB. Interestingly, it was found that the driver genes CCND1 and CCND2 are mutually exclusive between the patients. Aside from the malignant PC a small fraction of healthy-like polyclonal PC was detected in most patients, with higher variability in the PRMM cohort after the first line of treatment (FIG. 1C). In order to further characterize the transcriptional heterogeneity of the malignant PC of each patient, the metacell clustering was applied on the PC from each patient together with a control dataset of healthy PC (FIG. 2H). In order to precisely define the molecular characteristic of each patient malignant PC, all healthy-like PC were excluded based on the metacell assignment and defined transcriptionally homogenous clones for each patient (Methods), the present inventors further validated that each clone homogenously expresses a single VDJ arrangement. The present inventors analyzed if common driver genes are associated with malignant PC of a specific patient group and found no significant differences (p >0.05, fisher exact test) in the expression of previously described driver genes across the NDMM and PRMM patient groups (FIG. 1D). Additionally, no significant difference was observed in the clonality of the NDMM and PRMM groups, with 6/15 of the NDMM and 12/34 of the PRMM malignant plasma cells containing more than one transcriptional sub-clone (FIG. 1E, Methods). Together, the data define the molecular signature and driver genes of malignant PC within NDMM and PRMM patients showing no significant difference between the two groups.

### Example 2

### Refractory and Resistant MM Patients are Characterized by Unique Stress Pathways

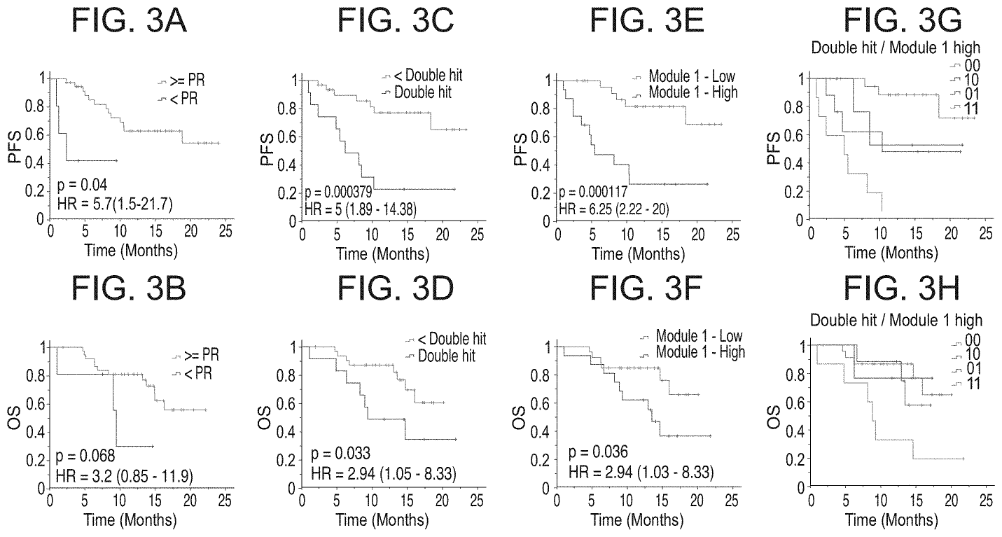

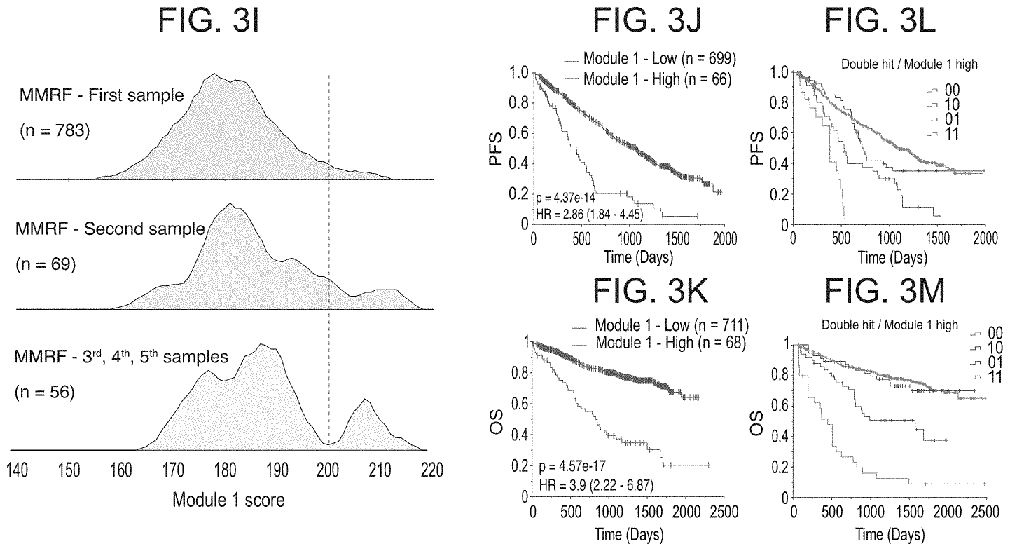

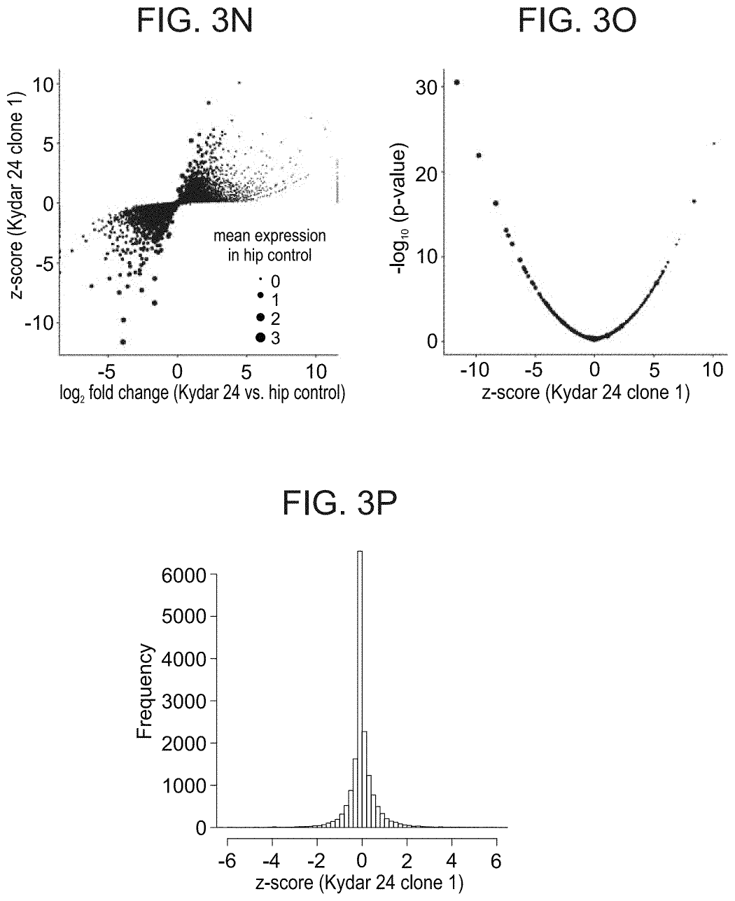

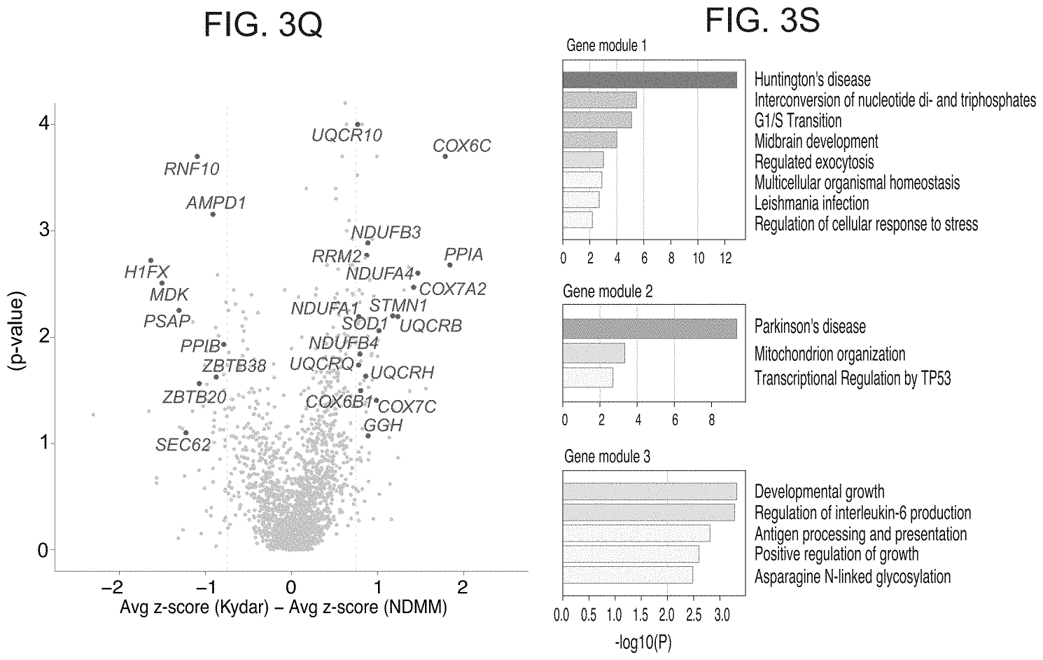

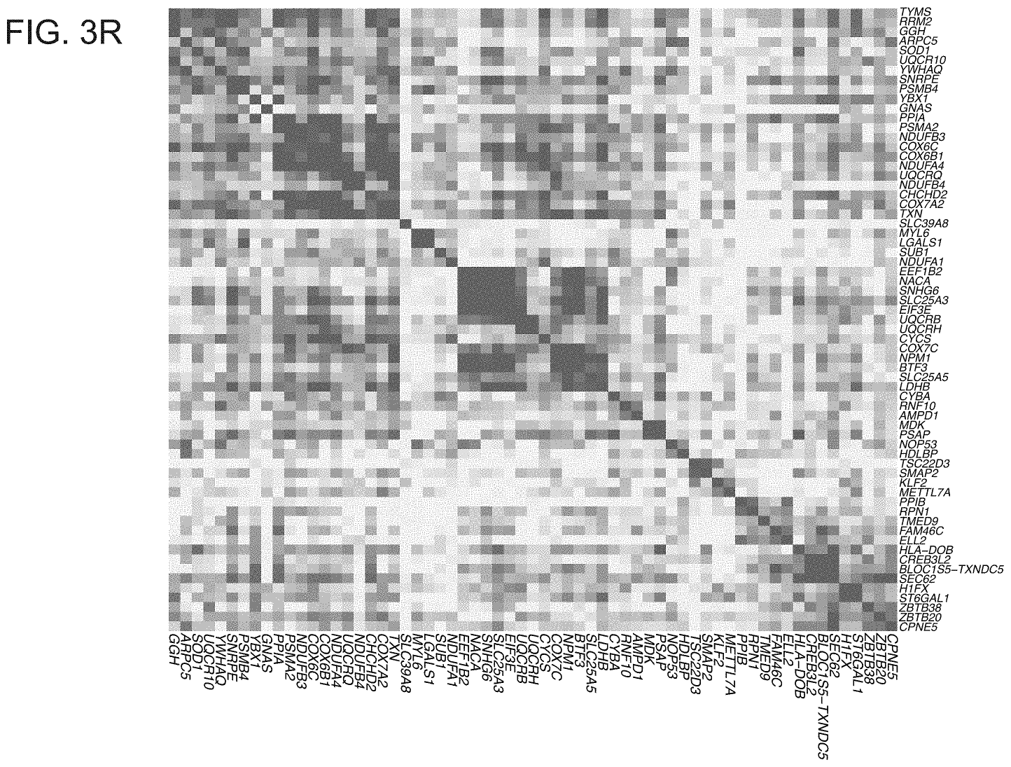

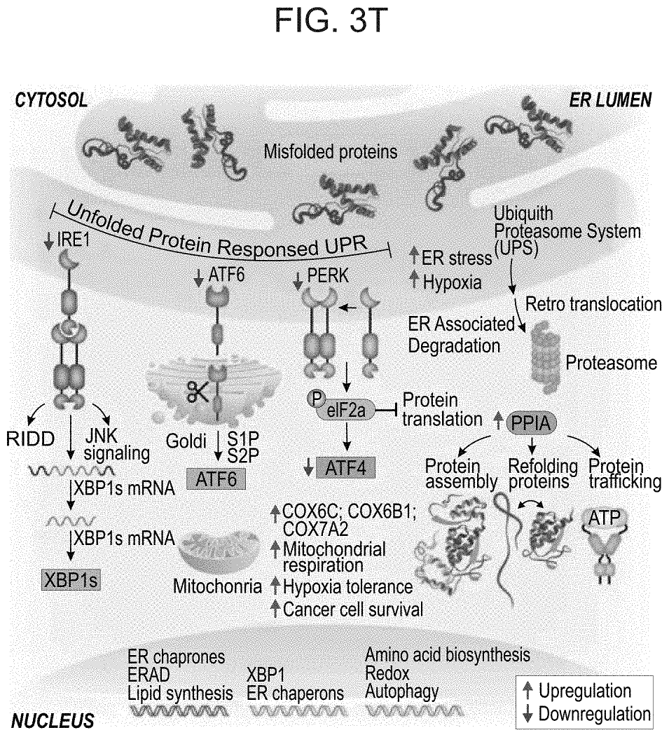

It was hypothesized that dedicated molecular pathways define the resistance mechanisms of malignant PC of PRMM patients, that have not yet been defined in bulk analysis or single cell analysis of NDMM patients. Bootstrapping on the t-statistic of the z-scores of gene expression was used in order to define differential genes between PRMM and NDMM patients (FIG. 2A and FIGS. 3N-Q; Method). 66 genes were found with p-value <0.05, hierarchical clustering of these genes resulted in a clustering structure consisting of 3 gene modules, distinguishing the NDMM and PRMM malignant PC state. Module 1 signature is defined by low expressed genes in the NDMM cohort and divides the PRMM patients into two groups, patients with low expression of module1 (Group 1) versus patients with high expression (Group 2) (FIGS. 2A-D and FIG. 3R). The genes in module 1 are highly enriched for specific pathways; mitochondrial stress genes COX6C, COX7A2, ER and UPR pathway genes PPIA, STMN1, oxidative stress SOD1, TXN, and the proteasome pathway PSMB4, PSMA2 (FIGS. 2A-B and FIGS. 3S-T). Projection of the modules score on the 2D patient map, defines a specific location for the PRMM patients expressing high level of module 1 (FIGS. 2C-D). These PRMM patients overexpressing module 1 are also characterized by down regulation of module 3. The present inventors evaluated if the difference in this signature is associated with either the refractory patients (cohort A) or relapsed patients (cohort B), but this resulted in no-significant enrichment (p-value=0.60, two sided t-test). Together, the present results describe a novel MM resistance signature that defines a subset of PRMM patients, including perturbation in mitochondrial stress genes, the ER and UPR pathway, and the proteasome machinery.

### Example 3

### Signatures of PRMM Patients can Predict Clinical Response to DARA-KDR Treatment

The combination of daratumumab (anti-CD38), selective proteasome inhibitor (carfilzomib), lenalidomide, and dexamethasone (DARA-KRD) is a highly effective quadruple anti-myeloma regimen, which is actively investigated in several phase 1 and 2 clinical trials as a first line treatment^{27,28,29}. Treatment emergent adverse events (TEAEs) occurred in 38 (93%) of the patients, and 5 (12%) of the patients discontinued study regimen due to adverse events. The most frequent (occurring in >10% of patients). The response evaluable analysis set included 41 patients who received at least 1 cycle of DARA-KRD and had at least one post baseline response assessment. The overall response rate (ORR) was 88% (cohort A: 85%, cohort B: 76%), 13% achieved complete response (CR) or better, 50% achieved very good partial response (VGPR), and 25% achieved partial response (PR) (FIGS. 4F-G). Median progression-free survival (PFS) was 14.7 months for the entire cohort (FIG. 4F-H) and was not reached for patients who achieved best response of partial response (PR) or better and was 1.8 month for patients who achieved less than PR (FIG. 3A) (HR 5.7, CI 95% 1.5-21.7, p=0.04). Median overall survival (OS) was not reached for the entire cohort (FIG. 41) and for patients who achieve PR and better, median OS was 9.3 (CI 95% 2.4-16.3) months for patients who failed to achieve PR (HR 3.2, CI 95% 0.85-11.9, p=0.068) and achieved less than PR (FIG. 3B). Due to the selection of patients with poor response to first line therapy, all KYDAR patients are considered at high risk for relapse, 67% also demonstrated high risk cytogenetic aberrations and 27% had more than 1 high-risk aberration (i.e. double hit myeloma). To test the effect of genetic aberrations, as detected by FISH, on patient outcome the patients were divided to double-hit myeloma and patients with no more than 1 high risk aberration. Median PFS was not reached for patients with up to 1 genetic risk aberration and was 4.9 months for patients with at least two genetic hits (HR 5, CI 95% 1.89-14.38, p=0.000379) (FIGS. 3C-D). The molecular signatures were analyzed in order to differentiate the PRMM cohort from NDMM patients is correlated to patient outcome. It was found that module 1 is a highly predictive molecular signature of unresponsive MM patients, exceeding the genetic risk stratification, with PFS 4.3 months and OS 9.3 months for patients with high module 1, both not reached for patients with low module 1 score (FIGS. 3E-F). By applying multivariate analysis, it was found that the molecular signature of module 1 and the genetic risk are not interacting covariates and the integration of these two variants significantly improved the predictive power (Cox regression p-value=0.022) (FIGS. 3G-H). Finally, the question of how prevalent is gene module 1 in newly diagnosed or relapsed MM patients and if it holds predictive power in larger independent data sets. The average RNA expression of module 1 genes was analyzed in 908 MM patients from the MMRF CoMMpass dataset. It was found that in MMRF patients prior to any treatment the average module 1 expression followed a normal distribution with no apparent sub populations, but when examining patients after treatment or patients after multiple relapses a gradient increase was detected in module 1 with a clear bi-model distribution (FIG. 3I). FIGS. 3J-K relate to CoMMpass patients with module 1 high expression had several clinical and biologic poor risk factors. These included worse renal function and poorer ECOG performance, worse ISS and R-ISS. Noteworthy, module 1 high patients had higher rate of poor cytogenetics, i.e double hit myeloma occurred in 38% of module 1 high versus 8% among module 1 low (p=8.6E−18). Strikingly, early (<18 months) progression occurred in 72% versus 41% in patients with high versus low module 1 patients, respectively Furthermore, the present inventors validated the significant contribution of module 1 beyond that of double hit cytogenetics in the CoMMpass dataset and R-ISS (not shown). In a multivariate model, including age, ISS, creatinine and high-risk FISH, module 1 remained a significant predictor of OS (HR 4.2, p=3.5E-14) and PFS (HR 2.9, p=2.9E-10) (FIGS. 3L-M and not shown). These results highlight the prognostic power of the module 1 gene signature in stratifying high-risk MM patients into ultra-resistant myeloma patients, both in PRMM as well as in advanced relapses after failure of multiple lines of therapy.

### Example 4

### Molecular Pathways of Broadly Resistant MM Patients

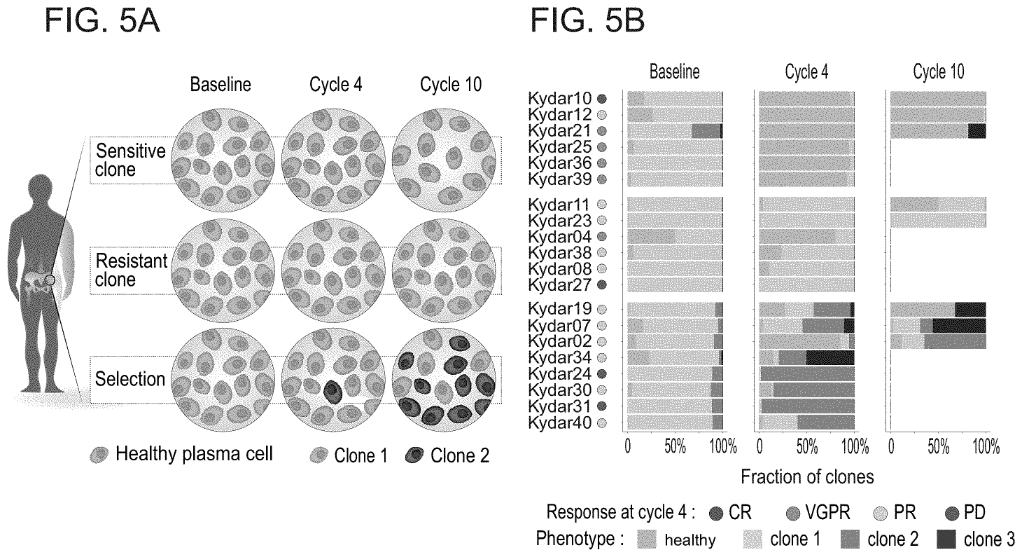

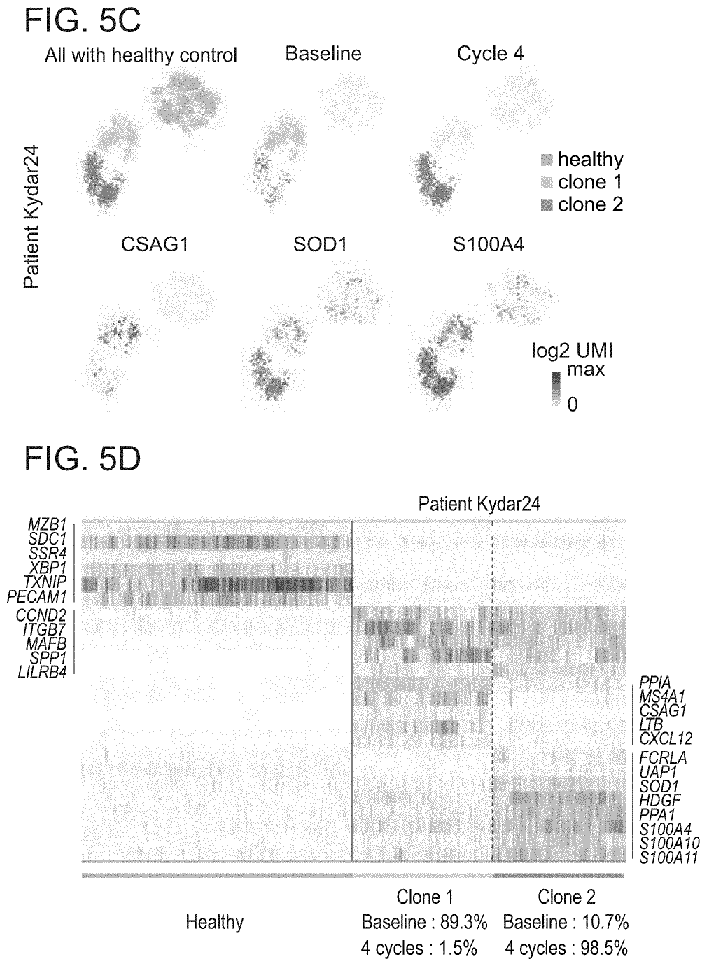

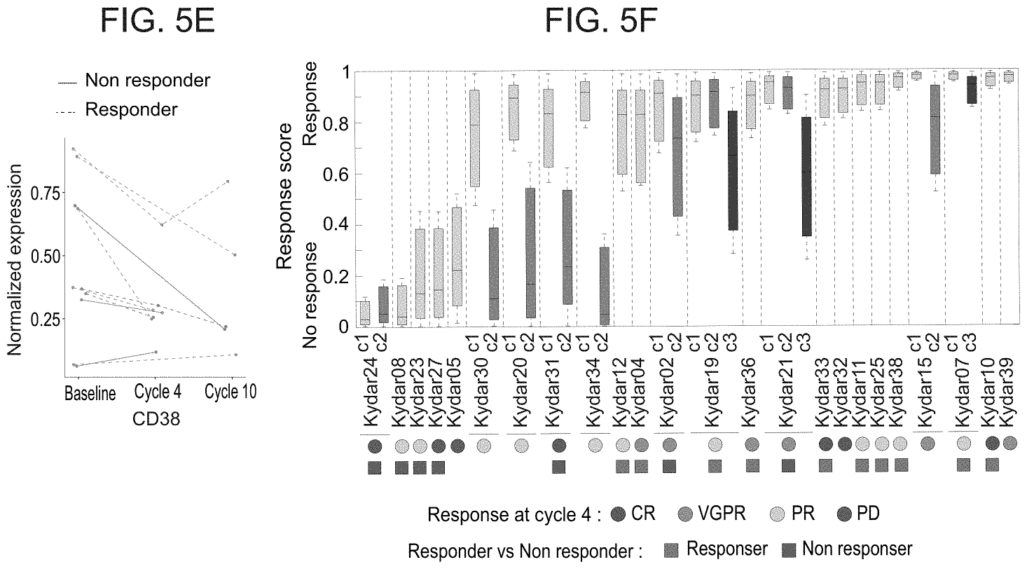

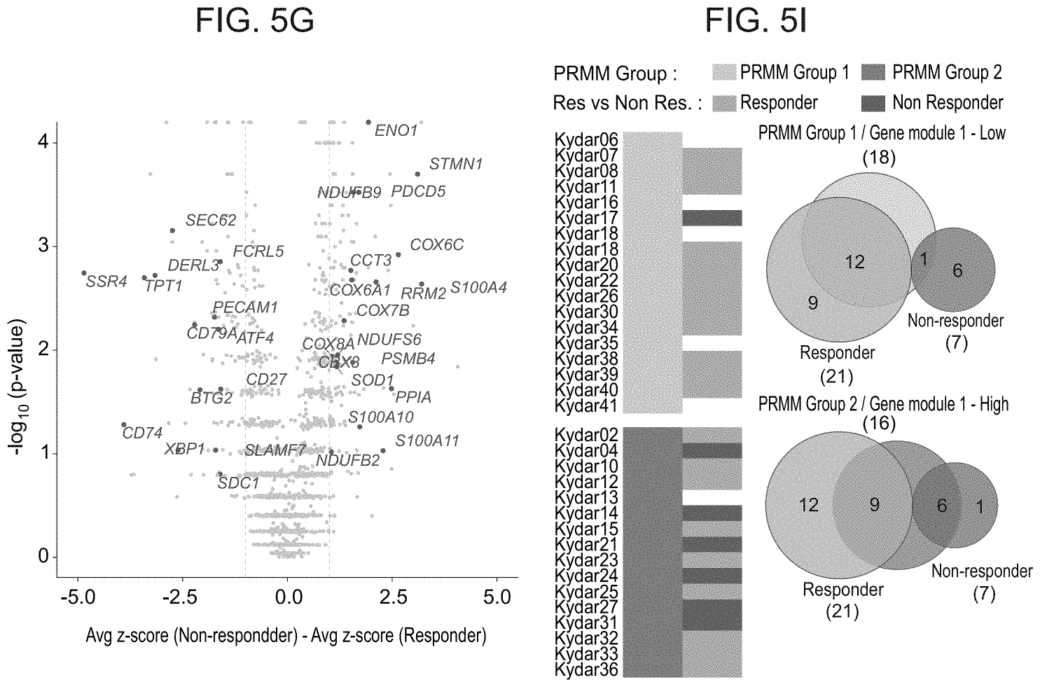

New therapeutic agents, and combinations have increased the survival of MM patients. However, most patients initially respond to upfront therapy, but later relapse³⁰. Although all PRMM patients have high-risk myeloma, most showed durable response to the quadruple anti-myeloma regimen, yet 7 out of 34 patients with baseline scRNA-seq data showed poor or transient response to the treatment, the present inventors defined non-responders as patients who failed to achieve PR at 6 months from study enrollment. To better characterize the molecular pathways associated with these broadly resistant MM patients, bootstrapping on the t-statistic of the z-scores of gene expression was used in order to define differential genes between the malignant PC of the PRMM responder and non-responder patient groups. 133 genes were found with a p-value <0.05, that can be separated into two major clusters, distinguishing PRMM responder and non-responder patient groups, with NDMM patients showing similar pattern as PRMM responder group (FIG. 4A and FIG. 5F). These gene signatures highlight putative MM resistance pathways, including upregulation of genes associated with immune regulation, proteasome, apoptotic and ER-stress pathways, e.g. Cyclophilin A (PPIA) and the programmed cell death protein 5 (PDCD5), creating an elaborated signature and potential target list of pathways and escape mechanisms from the most advanced combination therapy (FIGS. 4B-C). Several candidate markers were found that are highly differential between the responder versus non-responder patients. These include downregulation of multiple genes associated to PC function; ATF4, CD38, FCRL5 (FIG. 4B). As expected, CD38, the target of daratumumab, the immunotherapy used in this clinical trial, is downregulated in the non-responsive patient cohort^{31,32}. Additionally, the non-responder group had significantly up regulated the proteasome genes PSMD4, PSMB4 (FIG. 4A), suggesting a potential escape mechanism from the proteasome inhibitor in these patients^{33,16,14}. Also observed was a significant upregulation of mitochondrial stress genes (e.g. COX8A, COX6A1, and COX6C) in the non-responder compared to responder patients (FIG. 4A), suggesting that perturbation of respiratory activity of the mitochondria may increase survival of the malignant PC³⁴.

The function and transcriptional phenotype of the non-responder group is significantly different from the healthy donors, responder group and NDMM patients. This includes upregulation of potential escape mechanism and downregulation of PC canonical genes. Gene ontology enrichment analysis revealed increase in nucleoside metabolism (resistance signature 1, up regulated) and decrease in protein processing in the ER including intrinsic apoptotic signaling in response to ER stress (resistance signature 2, down regulated) (FIG. 4C). 2D projection of these signatures on the PC patient map largely overlap the territory of the PRMM resistant signature, suggesting the two sets of genes and patients are overlapping (FIG. 4D). Statistical analysis shows that this signature significantly overlaps the tumor resistant signature of the PRMM patients (p=3.5×10⁻⁷, hyper geometric test; FIG. 5H), as well as a significant overlap (6 of 7, p=0.029, hyper geometric test) in the KYDAR non-responder patient group and the PRMM patients expressing the module 1 signature (FIG. 5I). Considering that baseline (pre-treatment) PRMM malignant PC are characterized by a unique transcriptional signature, it was hypothesized that such data maybe effective for prediction of patient response to treatment. A shallow neural network classifier was trained, using 10 hidden layers, with a leave-one-out cross-validation strategy, to predict patient response to treatment after 4 months (FIG. 4E; Methods). The present model successfully predicts patient outcome while excluding the evaluated patient data to train the model, showing that the resistance mechanism detected are shared between patients and can be generalized to a larger cohort of patients. The present results show that the scRNA transcriptional data is highly correlated with clinical outcome and holds potential for use as companion diagnostic for MM and especially PRMM patients.

### Example 5

### Longitudinal Single Cell Clonal Dynamics of MM Patients' Response to Treatment

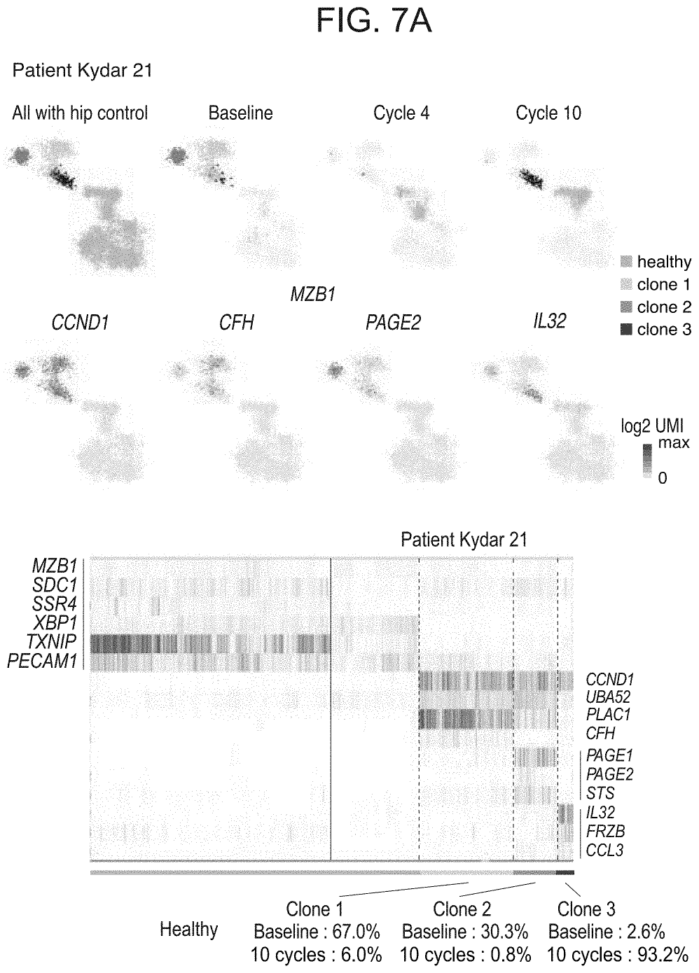

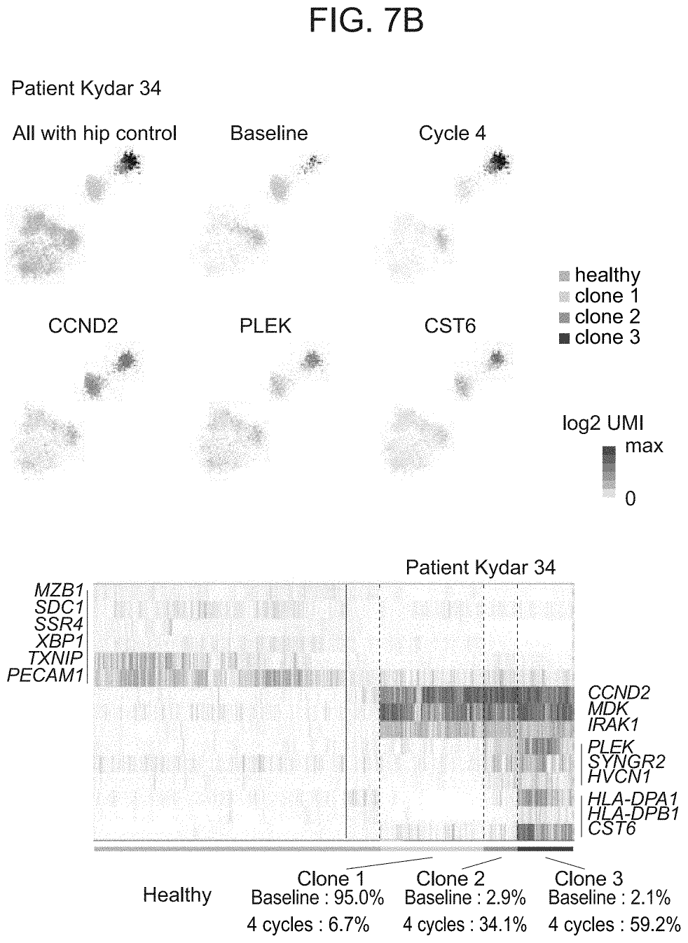

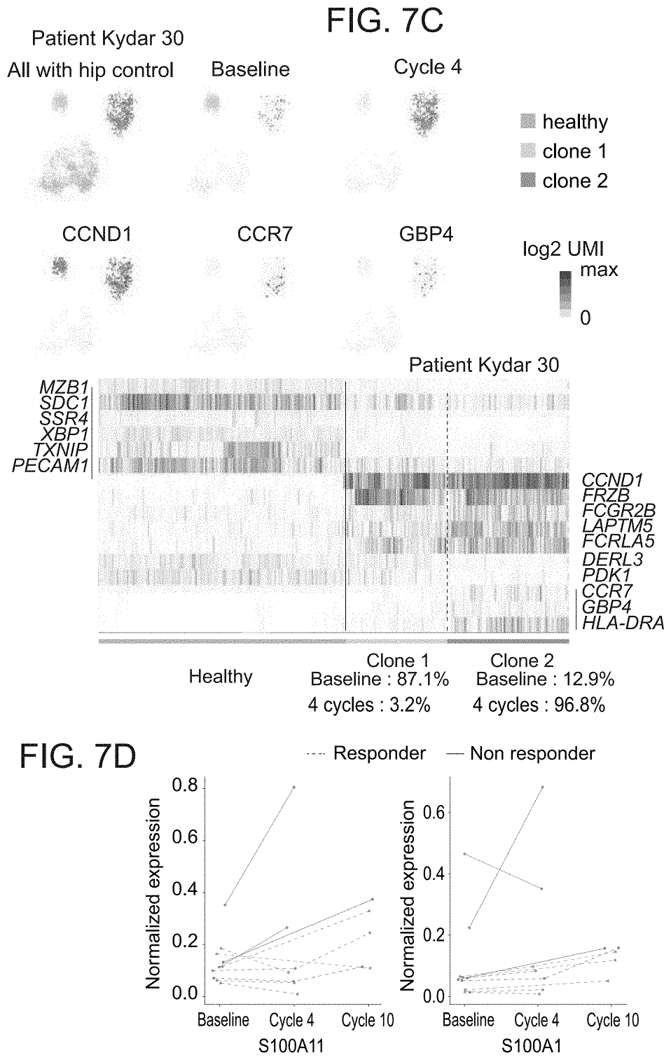

In addition to patients who failed to achieve very good response, many others who showed initial response later relapsed. Clonal evolution is a major driving force in tumor resistance and progression³⁵. Currently, the pathways and mechanisms of resistance, driving clonal selection in MM patients are only partly understood³⁶. To address this important question, a longitudinal single cell analysis was performed on PRMM patients' PC before and after treatment (at cycle 4 and cycle 10, FIGS. 6F-G) combined with clonal analysis using the B cell receptor sequence of each transcriptional clone. The present analysis of the transcriptional clonal dynamics in patients identifies three main trajectories: patients with sensitive PC clones or clone that respond to treatment with >90% of the malignant PC replaced with healthy-like PC (6 patients, 30%), patients with a resistant clone that did not respond or only very partly responded to treatment >50% of the malignant PC are found in the BM post treatment (6 patients, 30%) and patients with clonal selection, indicating that the major clone is replaced by a small or undetectable clone at baseline (8 patients, 40%) (FIGS. 5A-B). These results are on a par with the clinical response of the patients, except for patients Kydar12 and Kydar04, but add detailed information of the molecular malignant PC dynamics, pre- and post-treatment. Patient KYDAR 24, a clinical non-responder, is an example of selective clonal evolution dynamics with an evolving transcriptional state throughout the clinical trial. A 2D map of the patient malignant PC, including healthy PC as reference shows the transition from clone 1 at baseline with high expression of CSAG1 and MS4A1 genes to clone 2 at cycle 4 that downregulated CSAG1 and MS4A1, and upregulated SOD1, S100A4 (10.7% in baseline vs 98.5% in cycle 4) (FIGS. 5C-D). These clones share the same BCR suggesting a shared ancestor (FIG. 6H). Importantly the major MM drivers such as CCND2, ITGB7, and MAFB are shared between both transcriptional clones. The present longitudinal analysis revealed clonal dynamics and transcriptional changes in 9 out of 20 patients, in all cases main MM drivers were shared between the different clones with unique gene expression alterations per patient (FIGS. 7A-C).

Using pairwise t-test analyses of the dynamic data common molecular changes shared between resistant clones across patients were defined. This defined a list of genes with significant changes between the sensitive versus resistant clones. One of the genes showing consistent downregulation from the original clone to the emerging resistant clones is CD38 which is the target of Daratumumab, and previously shown to be downregulated in response to treatment^{31,32} (FIG. 5E). Also observed was upregulation of genes that are associated with myeloid cells program such as S100A0, S100A11 (FIG. 7D)³⁷. Finally, a neural network was applied trained on the pre-treatment malignant PC of the patients on all the clones collected longitudinally from PRMM patients. For several patients, with initial clones classified as responders by the model, a switch was observed to a non-responder clone: KYDAR 30, KYDAR 20, KYDAR 31, KYDAR 34 KYDAR 19, KYDAR 21 and KYDAR 15 (FIG. 5F). These transcriptional clones were not introduced as training data to the model and emerged following treatment, showing that the acquisition of therapeutic resistance may occur in multiple stages, or on small clones that later define the patient outcome. In summary, present data demonstrate that dynamic single cell analysis is an important molecular microscope to dynamically define patients' response to treatment and the genes and pathways associated with tumor resistance.

### Example 6

### PPIA a New Target for MM Patients with Broad Resistance Mechanisms

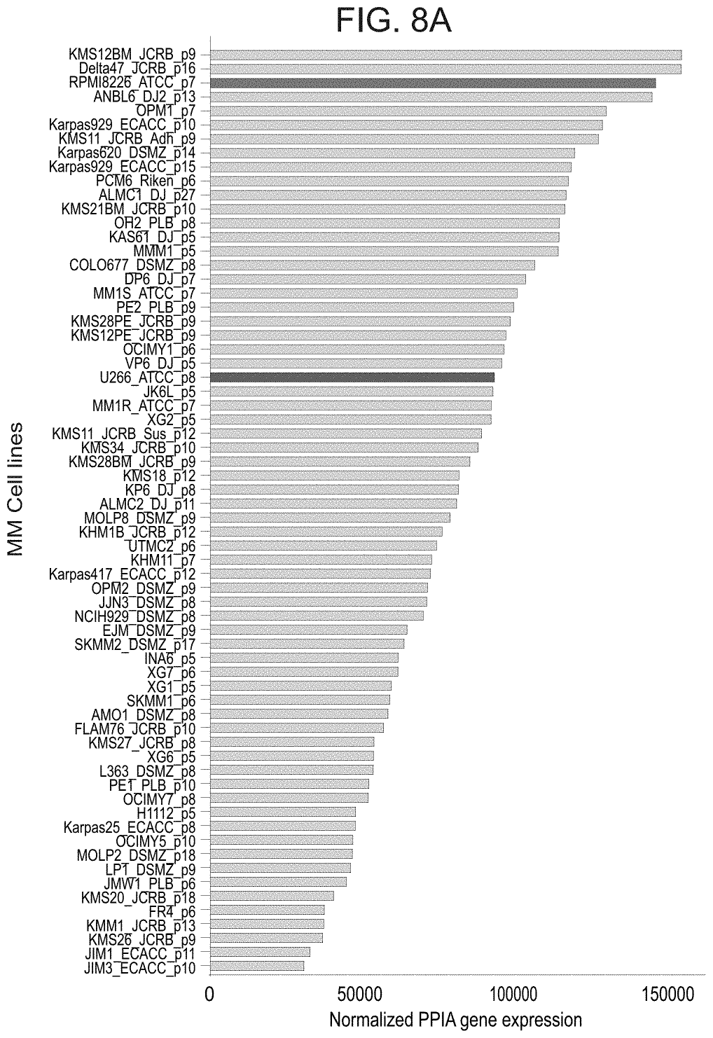

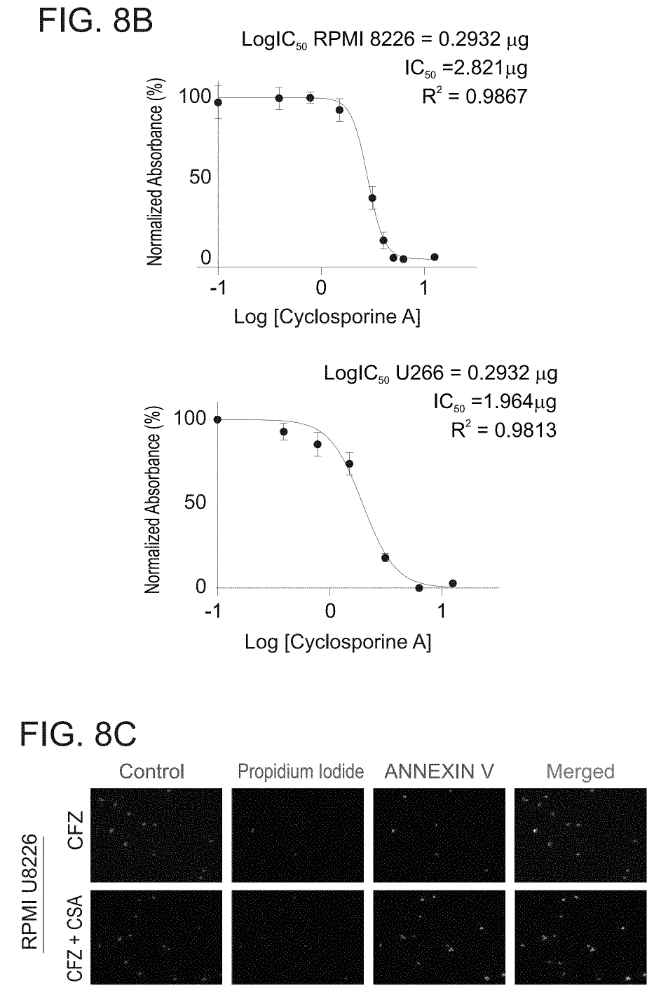

Cyclophilin A (PPIA gene) is a peptidyl-prolyl cis-trans isomerase enzyme that accelerates protein folding³⁸. Present data characterize PPIA as a potential MM resistance gene, which is highly expressed in both the PRMM Group 2 (FIGS. 2A-B) and the KYDAR non-responder patients (FIGS. 4A-B). It was hypothesized that PPIA may function as a protective resistance gene in MM malignant cells, potentially by accelerating protein folding pathways and reducing stress associated to proteasome inhibitors³⁹³⁸ (FIG. 6A). In order to test whether PPIA is merely a marker for highly resistant patients or has any causal role in MM resistance to proteasome inhibitors, Cyclosporine A (CsA) a known inhibitor of PPIA⁴⁰, was used in a series of in vitro experiments to explore the potential efficacy of CsA and proteasome inhibitors combinations. The database for MM cell lines that express high level PPIA was searched and RPMI-8226 and U266B were found as MM cell lines expressing relatively high levels of PPIA (FIG. 8A). The proliferation of these MM cell lines was measured following CsA treatment and the half maximal inhibitory concentration (IC50) of CsA was determined: 2.8 μg for RPMI 8226 and 2.0 ug for U266 in an MTS proliferation assay (FIG. 8B; Methods). The inhibitory activity of CsA inhibitory was determined on MM resistant cell lines so as to synergize with the activity of first line MM proteasome inhibitors. MM cells were seeded and treated with either Carfilzomib, CsA (IC50) and in combination therapy. Following 48 hours, cells were quantified by an MTS proliferation assay. The combined therapy of CsA and Carfilzomib was significantly more effective than Carfilzomib as a monotherapy (FIG. 6B). To further evaluate the significance of these findings, the effects of these drugs was measured in apoptosis induction of MM. Apoptosis was measured by Propidium Iodide, DAPI and Annexin V FITC staining which showed a dramatic increase of cell apoptosis in the combination therapy setting compared to Carfilzomib or CsA as a monotherapy (FIG. 6C-E). In conclusion, it was find that PPIA is correlated with broad resistance signatures of MM, and that CsA, a PPIA inhibitor, synergizes with PI to overcome resistance mechanisms of MM cell lines.

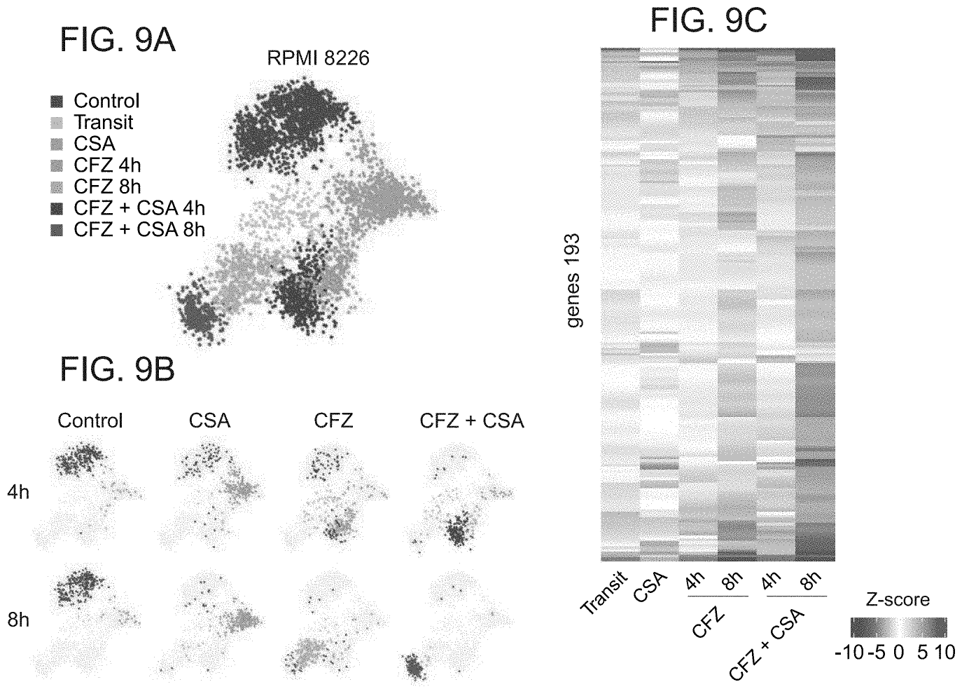

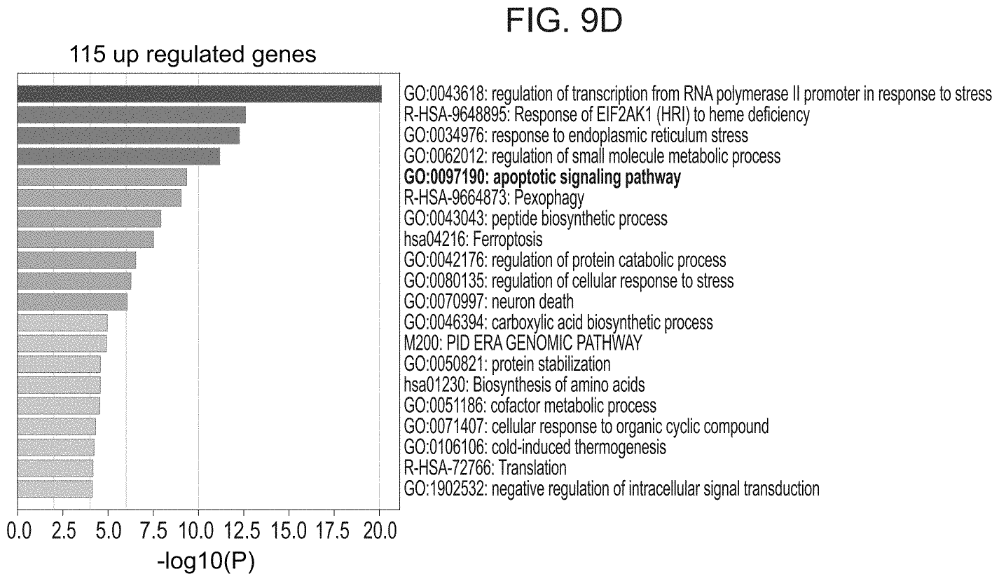

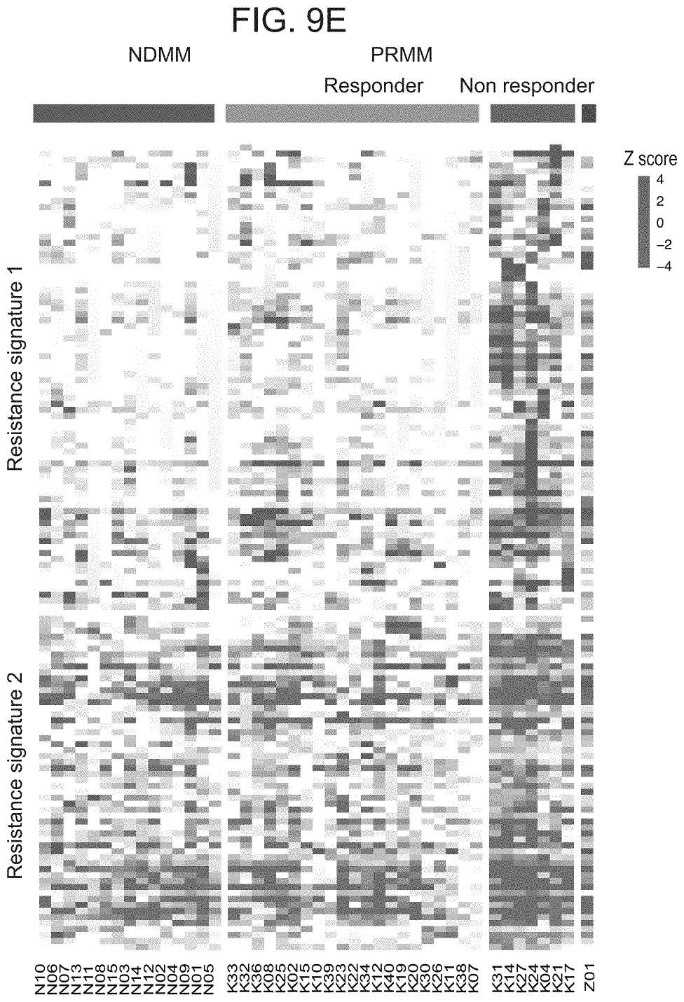

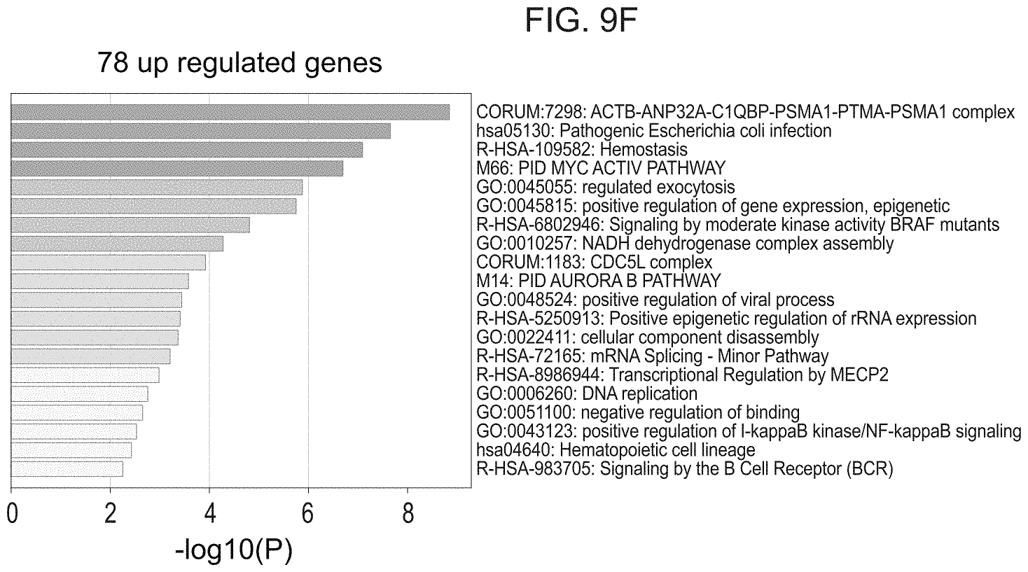

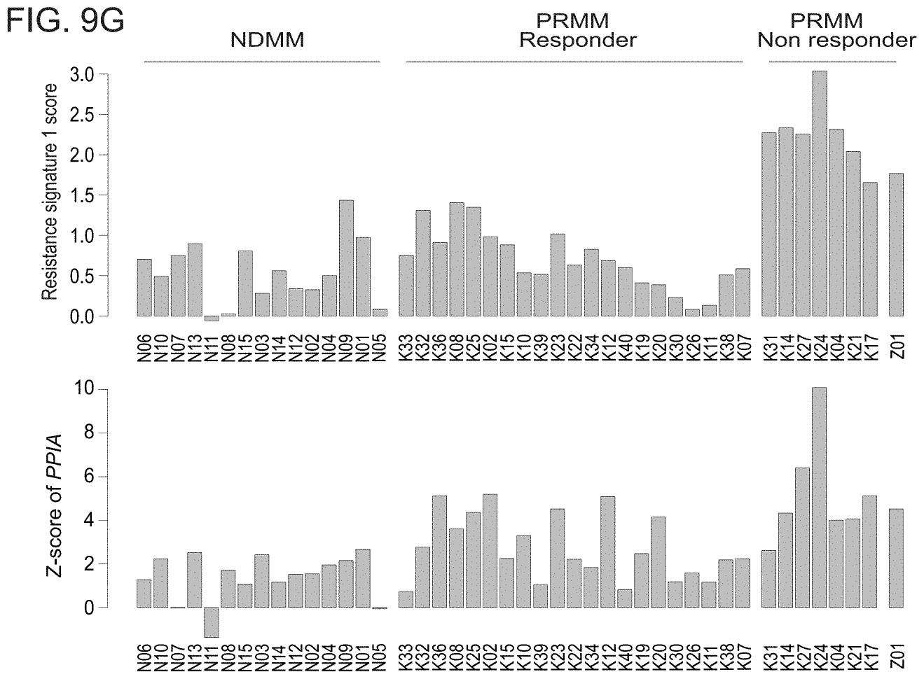

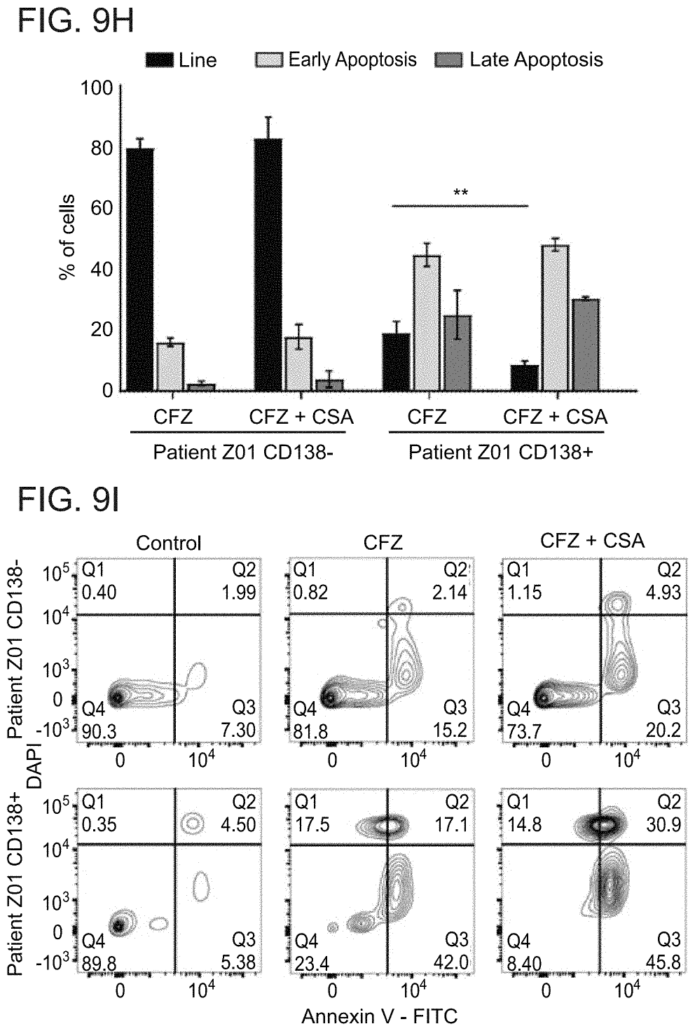

To further define the molecular mechanisms underlining the response to CFZ and the synergistic effect of CFZ+CsA, the present inventors performed scRNA-seq analysis of RPMI-8226 treated with CFZ, CsA and their combination 4- and 8-hours post treatment. Metacell analysis revealed 7 clusters, cluster 1 was enriched with mock-treated, healthy cells (control), cluster 2 contained cells in transition from cluster 1 to pre-apoptotic states of clusters 4-7. Cluster 3 was enriched with cells treated with CsA alone, clusters 4-5 contained cells from 4 h and 8 h treated with CFZ and clusters 6-7 was enriched for cell treated in the CFZ+CsA (FIGS. 9A-B). Differential gene expression analysis revealed that clusters 4-7 were defined by induction of genes involved in stress response and particularly ER stress response (e.g. ATF3, ATF4 and BAG3), as well as apoptotic signaling pathways (e.g. DEDD2, DDIT4, GAS5, and more) while down regulating housekeeping and metabolic genes such as LDHA (FIGS. 9C-D). Interestingly, cells treated with the combination of CFZ+CsA expressed higher levels of these signatures with almost all cells reaching the apoptotic stage compared to CFZ alone, supporting the synergistic effect observed in the functional assays.

Finally, it was evaluated whether this synergistic effect is also relevant to PI resistant MM patients. For this end, the present inventors collected BM sample from a highly resistant PRMM patient (Z01, Methods) and sorted the cells for MARS-seq analysis, confirming the patient is also positive for the resistance signature 1 (FIGS. 9E-G). Additionally, the present inventors seeded from the same patient CD138 positive and CD138 negative cells with medium supplemented with: CFZ, CsA or CFZ+CsA (Methods). The present inventors evaluated the patient ex vivo response to these treatments by FACS analysis of apoptotic cells using DAPI and Annexin-V staining. Mock control treated cells were viable and healthy, while the present inventors observed a synergistic effect of CFZ+CsA with only 8.4% live cells compared to 23.4% live cells when treating with CFZ alone (P<0.004) (FIGS. 9H-I). In conclusion, the present inventors find that PPIA is directly involved in PI resistance, and that CsA, a PPIA inhibitor, synergizes with PI to overcome resistance mechanisms of MM.

### Example 7

### Cyclosporine in Combination with Carfilzomib and Dexamethasone in Relapsed Multiple Myeloma Refractory to Carfilzomib and High Expression of PPIA Gene in Myeloma Cells

A phase 1, open-label, single-arm, prospective, single center study evaluates the safety, tolerability and efficacy of cyclosporine in combination with carfilzomib and dexamethasone in patients with relapsed and refractory multiple myeloma (RRMM). The patients consist of adult men and women who have a confirmed diagnosis of RRMM, who have received at least two prior lines of therapy including carfilzomib, and were non responsive or refractory to carfilzomib as specified below, and who were found to have elevated expression of Peptidylprolyl Isomerase A (PPIA) in scRNA sequencing of their myeloma cells, and who meet other protocol outlined eligibility criteria.

The patients are treated with cyclosporine with dose titration based on repeated determinations of whole blood concentrations to achieve a target trough concentration of 250 ng/mL, in combination with carfilzomib plus dexamethasone (Carfilzomib at 56 mg/m2 days 1,8,15 on a 28 d cycle. Dexamethasone 10 mg twice weekly.).

Patients may continue to receive treatment for 4 months or until disease progression or unacceptable toxicity, the earliest of them.

Subjects are followed for Adverse Events (AEs), clinical status-Overall response rate (ORR) as defined by International Myeloma Working Group (IMWG) criteria, Progression Free Survival (PFS), Duration of Response (DOR), Time to Progression (TTP), stringent Complete Response (sCR 0, Complete Response (CR), Very Goog Partial Response (VGPR), Partial Response (PR), Depth of Best Response (DpR), Time To Response (TTR), Progressive Disease (PD), Overall Survival (OS) and laboratory parameters for up to 4 months, unless they terminate early due to disease progression, unacceptable toxicity or due to meeting one of the withdrawal criteria

### Inclusion Criteria:

Patients must meet all of the following inclusion criteria:
1. Male or female patients, 18 years of age or older.
2. Multiple myeloma diagnosed according to standard IMWG criteria.
3. Patients must have measurable disease defined by at least one of the following three measurements:
   Serum M-protein 1 g/dL (10 g/L).
   Urine M-protein 200 mg/24 hours.
   Serum free light chain assay: involved free light chain level at least 100 mg/L, provided that the serum free light chain ratio is abnormal.
4. Patients received one or two prior lines of therapy which must have included bortezomib, lenalidomide-and daratumumab.
5. Patient received carfilzomib-based therapy either as their most recent line of therapy and within 3 months from study enrolment, and either failed to achieve a minor response after completing 2 cycles of carfilzomib based therapy, or are refractory to treatment.
6. Patients were found to have a high-expression level of PPIA, >1.2 unique RNA molecules (UMI) per cell on average, by scRNA sequencing of their myeloma cells from bone marrow aspiration sample at study screening.
7. Patients must meet the following clinical laboratory criteria:
   Absolute neutrophil count (ANC) >1,000/mm3 and platelet count≥75,000/mm3. Platelet transfusions to help patients meet eligibility criteria are not allowed within 3 days of enrolment.
   Total bilirubin 1.5 the upper limit of the normal range (ULN).
   Alanine aminotransferase (ALT) and aspartate aminotransferase (AST)<3 ULN.
   Calculated creatinine clearance >45 mL/min
8. Eastern Cooperative Oncology Group (ECOG) performance status of 0, 1 or 2.
9. Female patients who:
   Are postmenopausal for at least 24 months before the screening visit, OR
   Are surgically sterile, OR
   Who are of childbearing potential, and agree to practice two effective methods of contraception (1 highly effective method and 1 additional effective method) at the same time, from the time of signing the informed consent through 90 days after the last dose of study treatment, OR agree to completely abstain from heterosexual intercourse. Females of childbearing potential (FCBP) must have a negative serum or urine pregnancy test with a sensitivity of at least 25 milli International Units/mL within 10 to 14 days of initiation of Cycle 1 and again within 24 hours of starting Cycle 1. FCBP must also agree to ongoing pregnancy testing. All patients must be counseled at a minimum of every 28 days about pregnancy precautions and risks of fetal exposure.
10. Male patients, even if surgically sterilized (i.e., status postvasectomy), who:
   Agree to completely abstain from heterosexual intercourse, OR
   Agree to practice effective barrier contraception (i.e., latex condom) during sexual contact with a FCBP, even if they have had a successful vasectomy, throughout the entire study treatment period and through 4 months after the last dose of study treatment.
11. Voluntary written consent must be given before performance of any study-related procedure not part of standard medical care, with the understanding that consent may be withdrawn by the patient at any time without prejudice to future medical care.
12. Patient is willing and able to adhere to the study visit schedule and other protocol requirements.

Exclusion Criteria:
Patients meeting any of the following exclusion criteria are not eligible to participate in the study:
1. Patient underwent an allogeneic transplantation.
2. Major surgery within 14 days before enrolment.
3. Central nervous system involvement
4. Concomitant use of any other antineoplastic treatment with activity against MM (with the exception of ≤40 mg Dexamethasone per day or equivalent for no longer than 4 days).
5. Anti-myeloma therapy as follows prior to screening bone marrow aspiration:
a. Targeted therapy, within 14 days or at least 5 half-lives, whichever is less;
b. Monoclonal antibody treatment for multiple myeloma within 21 days;
c. Cytotoxic therapy within 14 days;
d. Proteasome inhibitor therapy within 14 days; note: no window is required for carfilzomib
e. Immunomodulatory agent therapy within 7 days.
f. Radiotherapy within 14 days (with the exception of radiotherapy for spinal cord compression or for pain control that should be discussed and approved by the sponsor-investigator prior to study enrolment). However, if the radiation portal covered ≤5% of the bone marrow reserve, the subject is eligible irrespective of the end date of radiotherapy.
6. Diagnosed or treated for another malignancy within 2 years before enrolment or previously diagnosed with another malignancy and have any evidence of residual disease. Patients with nonmelanoma skin cancer or carcinoma in situ of any type are not excluded if they have undergone complete resection.
7. Moderate to severe kidney injury (Calculated creatinine clearance ≤45 mL/min).
8. Severe liver disease (cirrhosis grade Child-Pugh B or C; significant hepatocellular or cholestatic liver injury).
9. Diagnosis of Waldenstrom's macroglobulinemia, POEMS (polyneuropathy, organomegaly, endocrinopathy, monoclonal gammopathy, and skin changes) syndrome, plasma cell leukaemia, primary amyloidosis, myelodysplastic syndrome, or myeloproliferative syndrome.
10. Evidence of current uncontrolled cardiovascular conditions, including uncontrolled hypertension, uncontrolled cardiac arrhythmias, symptomatic congestive heart failure, unstable angina, or myocardial infarction within the past 6 months.
11. Psychiatric illness/social situation that would limit compliance with study requirements.
12. Patient with a known diagnosis of Epilepsy.
13. Known allergy to any of the study medications, their analogues, or excipients in the various formulations of any agent.
14. Comorbid systemic illnesses or other severe concurrent disease which, in the judgment of the investigator, would make the patient inappropriate for entry into this study or interfere significantly with the proper assessment of safety and toxicity of the prescribed regimens.
15. Systemic treatment with strong inhibitors of Cytochrome P450 family 3, subfamily A (CYP3A) (clarithromycin, telithromycin, itraconazole, voriconazole, ketoconazole, nefazodone, posaconazole) or strong Cytochrome P450 (CYP3A), family 3, subfamily A inducers (rifampin, rifapentine, rifabutin, carbamazepine, phenytoin, phenobarbital), or use of Ginkgo biloba or St. John's wort within 14 days before enrolment in the study.
16. Infection requiring systemic antibiotic therapy or other serious infection within 14 days before enrolment.
17. Ongoing or active systemic infection, active hepatitis B virus infection, active hepatitis C infection, or known human immunodeficiency virus (HIV) positive.
18. Vaccination with live attenuated viruses (i.e. yellow fever injections, polio drops, chickenpox (herpes varicella) and shingles (herpes zoster) vaccines) within 30 days before enrolment.
19. Inability to swallow oral medication, inability or unwillingness to comply with the drug administration requirements, or known GI disease or planned gastrointestinal (GI) procedure that could interfere with the oral absorption or tolerance of treatment.
20. Failure to have fully recovered (ie, Grade 1 toxicity) from the reversible effects of prior chemotherapy (except for alopecia).
21. Patient has Grade 3 peripheral neuropathy during the screening period.
22. Participation in other clinical trials, including those with other investigational agents not included in this trial, within 30 days of the start of this trial and throughout the duration of this trial.
23. Patients that have previously been treated with Cyclosporine plus carfilzomib.
24. Female patients who are lactating or pregnant.
   The primary and secondary outcome measures are provided hereinbelow, each of which can be used as a measure of success.

Primary Outcome Measures:
1. Number of Participants With Abnormal Laboratory Values and/or Adverse Events That Are Related to Treatment parameters. [Time Frame: follow-up 2 years post study]
   Number of Participants With Abnormal Laboratory Values and/or Adverse Events That Are Related to Treatment parameters.

Secondary Outcome Measures:
1. Overall response rate ORR [Time Frame: follow-up 2 years post study]
   Proportion of patients who achieve a best overall response of stringent complete response, complete response, very good partial response, or partial response as defined using the IMWG criteria
2. Progression free survival PFS [Time Frame: follow-up 2 years post study]
   The time from first dose to the date of the first documented tumor progression or death due to any cause. PFS will be determined by an investigator, based upon laboratory data, as defined by the IMWG criteria
3. Duration of Response DOR [Time Frame: follow-up 2 years post study]
   The time between the date of first response to the date of the first objectively documented tumor progression as assessed by study steering committee according to modified IMWG criteria or death due to any cause prior to subsequent anti-cancer therapy.
4. Time to Response TTR [Time Frame: follow-up 2 years post study]
   The time from the first dose to the date of the first sCR, CR, VGPR, or PR.
5. Depth of Best Response (DpR) [Time Frame: follow-up 2 years post study]
   According to IMWG criteria.
6. Time to progression (TTP) [Time Frame: follow-up 2 years post study]
   Time from initiation of treatment to documented PD
7. Overall Survival (OS) [Time Frame: follow-up 2 years post study]
   Time between the date of first dose and the date of death due to any cause
8. Extramedullary progression [Time Frame: follow-up 2 years post study]
   Kaplan Meyer test will be applied to test for differences between the groups in Progression Free Survival
9. Extramedullary progression [Time Frame: follow-up 2 years post study]
   Kaplan Meyer test will be applied to test for differences between the groups in Overall Survival
10. Percentage of cyclosporine trough levels tests in acceptable range [Time Frame: follow-up 2 years post study]
   will be calculated for each patient.
11. Mean % of levels in acceptable range will be calculated for the efficacy population. [Time Frame: follow-up 2 years post study]

### Example 8

### Combined Treatment of MM Using a PPIA Inhibitor and a Proteasome Inhibitor-a Case Report

An 87 year old patient with a history of ischemic heart disease, hypertension, hypothyroidism, aortic aneurysm, and a/p CVA diagnosed with multiple myeloma on April 2018 with multiple bone lesions. Patient received treatment with bortezomib dexamethasone with a partial response as Myeloma progressed and on February 2019 he enrolled into the KYDAR clinical trial (NCT04065789) to receive carfilzomib, Lenalidomide, dexamethsoane and daratumumab combination. He completed 18 cycles of therapy and thereafter continued to receive datatumumab combined with cyclophosphamide post study. On August 2020 he experienced a biochemical progression, followed by clinical progression with worsening bone disease on PETCT (5 Oct. 2020). Myeloma monoclonal protein increased progressively: Lambda light chain 235 mg/dL & MSPIKE 8.3 g/L (May 2020) to Lambda LC 510 mg/L and MSPIKE of 11.8 g/dL (18 Oct. 2020).

On October 25′ 2020 he started therapy with IV carfilzomib at 56 mg/m2 one weekly together with PO cyclosporine A at 50 mg twice daily, as compassionate salvage therapy after receiving regulatory approval from the hospital ethics committee according to Israeli local regulations (29-Gimel procedure). The dose of cyclosporine was up-titrated up to 125 md twice daily, while monitoring cyclosporine blood levels (target level between 100 to 200 ng/mL) and renal function. The treatment was well tolerated. Under this treatment, Lambda decreased to a nadir of 263 mg/L and MSPIKE to 6.2 g/dL (1 Nov. 2020). He reported improvement in bone pain and wellbeing, a clinically significant result for an MM patient. He continued receiving this therapy until March 2021, at that time disease progressed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Kyle, R. A. & Rajkumar, S. V. Treatment of multiple myeloma: a comprehensive review. Clinical lymphoma & myeloma 9, 278-288 (2009).
2. Palumbo, A. & Anderson, K. Multiple myeloma. New England Journal of Medicine 364, 1046-1060 (2011).
3. Yan, Y. et al. The impact of response kinetics for multiple myeloma in the era of novel agents. Blood Adv. 3, 2895-2904 (2019).
4. Nijhof, I. S., van de Donk, N. W. C. J., Zweegman, S. & Lokhorst, H. M. Current and New Therapeutic Strategies for Relapsed and Refractory Multiple Myeloma: An Update. Drugs 78, 19-37 (2018).
5. Rajkumar, S. V. Multiple Myeloma: 2020 update on Diagnosis, Risk-stratification and Management. Am. J. Hematol. (2020). doi:10.1002/ajh.25791
6. Cohen, Y. C. et al. Primary failure of bortezomib in newly diagnosed multiple myeloma—understanding the magnitude, predictors, and significance. Leuk. Lymphoma 1-7 (2016). doi:10.3109/10428194.2015.1121258
7. Majithia, N. et al. Outcomes of primary refractory multiple myeloma and the impact of novel therapies. Am. J. Hematol. 90, 981-985 (2015).
8. Corre, J. et al. Early relapse after autologous transplant for myeloma is associated with poor survival regardless of cytogenetic risk. Haematologica haematol.2019.236588 (2019). doi:10.3324/haematol.2019.236588
9. Kumar, S. K. et al. Early relapse after autologous hematopoietic cell transplantation remains a poor prognostic factor in multiple myeloma but outcomes have improved over time. Leukemia 32, 986-995 (2018).
10. Kastritis, E. et al. Early Relapse post Autologous Transplant is Associated With Very Poor Survival and Identifies an Ultra High Risk Group of Myeloma Patients. Clin. Lymphoma Myeloma Leuk. 1-8 (2020). doi:10.1016/j.clml.2019.10.014
11. van de Donk, N. W. C. J. et al. Treatment of relapsed and refractory multiple myeloma in the era of novel agents. Cancer Treatment Reviews 37, 266-283 (2011).
12. T. Wallington-Beddoe, C. & W. Coghlan, D. Resistance Mechanisms to Novel Therapies in Myeloma. in Update on Multiple Myeloma (IntechOpen, 2019). doi:10.5772/intechopen.77004
13. Krishnan, S. R., Jaiswal, R., Brown, R. D., Luk, F. & Bebawy, M. Multiple myeloma and persistence of drug resistance in the age of novel drugs (Review). Int. J. Oncol. 49, 33-50 (2016).
14. Niewerth, D. et al. Molecular basis of resistance to proteasome inhibitors in hematological malignancies. Drug Resist. Updat. 18, 18-35 (2015).
15. Harding, T., Baughn, L., Kumar, S. & Van Ness, B. The future of myeloma precision medicine: integrating the compendium of known drug resistance mechanisms with emerging tumor profiling technologies. Leukemia 33, 863-883 (2019).
16. Papadas, A. & Asimakopoulos, F. Mechanisms of resistance in multiple myeloma. in Handbook of Experimental Pharmacology 249, 251-288 (Springer New York LLC, 2018).
17. Nikesitch, N. & Ling, S. C. W. Molecular mechanisms in multiple myeloma drug resistance. J Clin Pathol 69, 97-101 (2016).
18. Narayanan, S. et al. Targeting the ubiquitin-proteasome pathway to overcome anti-cancer drug resistance. Drug Resist. Updat. 48, 100663 (2020).
19. Vincenz, L., Jager, R., O'Dwyer, M. & Samali, A. Endoplasmic reticulum stress and the unfolded protein response: Targeting the achilles heel of multiple myeloma. Molecular Cancer Therapeutics 12, 831-843 (2013).
20. Ling, S. C. W. et al. Response of myeloma to the proteasome inhibitor bortezomib is correlated with the unfolded protein response regulator XBP-1. Haematologica 97, 64-72 (2012).
21. Klein, A. M. & Treutlein, B. Single cell analyses of development in the modern era. Development (Cambridge, England) 146, (2019).
22. Ledergor, G. et al. Single cell dissection of plasma cell heterogeneity in symptomatic and asymptomatic myeloma. Nat. Med. 24, 1867-1876 (2018).
23. Kawano, Y. et al. Targeting the bone marrow microenvironment in multiple myeloma. Immunol. Rev. 263, 160-172 (2015).
24. Cohen, M. et al. Lung Single-Cell Signaling Interaction Map Reveals Basophil Role in Macrophage Imprinting. Cell 175, 1031-1044.e18 (2018).
25. Medaglia, C. et al. Spatial reconstruction of immune niches by combining photoactivatable reporters and scRNA-seq. Science (80-.). 358, 1622-1626 (2017).
26. Baran, Y. et al. MetaCell: analysis of single cell RNA-seq data using k-NN graph partitions. bioRxiv 437665 (2018). doi:10.1101/437665
27. Jakubowiak, A. J. et al. Daratumumab (DARA) in combination with carfilzomib, lenalidomide, and dexamethasone (KRd) in patients (pts) with newly diagnosed multiple myeloma (MMY1001): An open-label, phase 1b study. J. Clin. Oncol. 35, 8000 (2017).
28. Landgren, O. et al. Bone Marrow-Based and Longitudinal Blood-Based MRD Tracking in Newly Diagnosed Multiple Myeloma Patients Treated with Daratumumab, Carfilzomib, Lenalidomide and Dexamethasone (DKRd): A Correlative and Clinical Phase II Study. Blood 132, 3281 (2018).
29. Chari, A. et al. Daratumumab (DARA) in Combination with Carfilzomib, Lenalidomide, and Dexamethasone (KRd) in Patients with Newly Diagnosed Multiple Myeloma (MMY1001): Updated Results from an Open-Label, Phase 1b Study. Blood 130, 3110 (2017).
30. Pawlyn, C. & Morgan, G. J. Evolutionary biology of high-risk multiple myeloma. Nature Reviews Cancer 17, 543-556 (2017).
31. Van De Donk, N. W. C. J. & Usmani, S. Z. CD38 antibodies in multiple myeloma: Mechanisms of action and modes of resistance. Frontiers in Immunology 9, (2018).
32. Krejcik, J. et al. Daratumumab depletes CD38+ immune regulatory cells, promotes T-cell expansion, and skews T-cell repertoire in multiple myeloma. Blood 128, 384-394 (2016).
33. Robak, P., Drozdz, I., Szemraj, J. & Robak, T. Drug resistance in multiple myeloma. Cancer Treatment Reviews 70, 199-208 (2018).
34. Tsvetkov, P. et al. Mitochondrial metabolism promotes adaptation to proteotoxic stress. Nat. Chem. Biol. 15, 681-689 (2019).
35. Greaves, M. & Maley, C. C. Clonal evolution in cancer. Nature 481, 306-313 (2012).
36. Keats, J. J. et al. Clonal competition with alternating dominance in multiple myeloma. Blood 120, 1067-1076 (2012).
37. Khoo, W. H. et al. A niche-dependent myeloid transcriptome signature defines dormant myeloma cells. Blood 134, 30-43 (2019).
38. Nigro, P., Pompilio, G. & Capogrossi, M. C. Cyclophilin A: A key player for human disease. Cell Death and Disease 4, e888-e888 (2013).
39. Kyu, J. C. et al. Overexpressed cyclophilin A in cancer cells renders resistance to hypoxia- and cisplatin-induced cell death. Cancer Res. 67, 3654-3662 (2007).
40. Matsuda, S. & Koyasu, S. Mechanisms of action of cyclosporine. Immunopharmacology 47, 119-125 (2000).
41. Kuiper, R. et al. A gene expression signature for high-risk multiple myeloma. Leukemia 26, 2406-2413 (2012).
42. Shaughnessy, J. D. et al. A validated gene expression model of high-risk multiple myeloma is defined by deregulated expression of genes mapping to chromosome 1. Blood 109, 2276-2284 (2007).
43. Nikesitch, N., Lee, J. M., Ling, S. & Roberts, T. L. Endoplasmic reticulum stress in the development of multiple myeloma and drug resistance. Clinical and Translational Immunology 7, (2018).
44. Lee, J. & Kim, S. S. An Overview of Cyclophilins in Human Cancers. The Journal of International Medical Research 38, (2010).
45. Jaitin, D. A. et al. Massively parallel single-cell RNA-seq for marker-free decomposition of tissues into cell types. Science (80-.). 343, 776-779 (2014).
46. Paul, F. et al. Transcriptional Heterogeneity and Lineage Commitment in Myeloid Progenitors. Cell 163, 1663-77 (2015).
47. Keren-Shaul, H. et al. A Unique Microglia Type Associated with Restricting Development of Alzheimer's Disease. Cell 169, 1276-1290.e17 (2017).
48. Kumar, S. et al. International Myeloma Working Group consensus criteria for response and minimal residual disease assessment in multiple myeloma. Lancet Oncol. 17, e328-e346 (2016).

What is claimed is:

1. A method of prognosing a subject diagnosed with multiple myeloma (MM), the method comprising determining in plasma cells (PC) of the subject a level of expression of at least one gene of Table A or A* and/or Table B or B*, wherein upregulation in at least one gene of Table A or A* and/or downregulation in at least one gens of Table B or B* as compared to expression of said genes in normal PC is indicative of poor prognosis, the method further comprising corroborating said prognosis with a Gold standard method.

2. A method of determining responsiveness to treatment with Daratumumab, Carfilzomib, Lenalidomide and Dexamethasone (DARA-KRD) in a subject diagnosed with multiple myeloma (MM), the method comprising:
treating the subject with Daratumumab, Carfilzomib, Lenalidomide and Dexamethasone (DARA-KRD); and
determining in plasma cells (PC) of the subject a level of expression of at least one gens of Table C and/or Table D, wherein upregulation in at least one gene of Table C and/or downregulation in at least one gene of Table D as compared to expression of said at least one gene in normal PC is indicative of responsiveness to treatment with Daratumumab, Carfilzomib, Lenalidomide and Dexamethasone (DARA-KRD).

3. The method of claim 2, wherein said subject exhibits primary resistance to a first line treatment.

4. The method of claim 1, wherein said subject exhibits early relapse of less than 18 months following improvement.

5. A method of treating a subject diagnosed with multiple myeloma (MM), the method comprising administering to the subject a therapeutically effective amount of a proteasome inhibitor and at least one agent which specifically down-regulates activity or expression of PPIA and/or RRM2, thereby treating the subject.

6. A method of treating a subject diagnosed with MM selected expressing intracellular PPIA and/or RRM2 above a predetermined threshold, the method comprising administering to the subject a therapeutically effective amount of at least one agent which specifically down-regulates activity or expression of PPIA and/or RRM2, thereby treating the subject.

7. The method of claim 5, wherein the subject exhibits primary resistance to a first line treatment, or the subject is diagnosed with Relapsed/Refractory Multiple Myeloma (RRMM) or the subject exhibits upregulation of said intracellular PPIA and/or RRM2 as compared to expression of same in normal PC or said subject exhibits early relapse following an anti-MM treatment of less than 18 months.

8. The method of claim 5, wherein said proteasome inhibitor is selected from the group consisting of Carfilzomib, Bortezomib and Ixazomib.

9. The method of claim 5, further comprising determining a level of said intracellular PPIA and/or RRM2 in PC of the subject, wherein upregulation of said intracellular PPIA and/or RRM2 as compared to expression of same in normal PC is indicative of responsiveness to treatment with said proteasome inhibitor and said agent.

10. The method of claim 5, wherein said at least one agent is PPIA inhibitor.

11. The method of claim 5, wherein said PPIA inhibitor is cyclosporine A (CSa).

12. The method of claim 5, wherein said RRM2 inhibitor is Cladribine.

13. The method of claim 5, wherein said PPIA inhibitor is cyclosporine A (CSa) and said RRM2 inhibitor is Cladribine; or
wherein said proteasome inhibitor is Carfilzomib, wherein said PPIA inhibitor is cyclosporine A (CsA) and optionally comprising dexamethasone.

14. The method of claim 6, wherein said proteasome inhibitor is selected from the group consisting of Carfilzomib, Bortezomib and Ixazomib.

15. The method of claim 6, further comprising determining a level of said intracellular PPIA and/or RRM2 in PC of the subject, wherein upregulation of said intracellular PPIA and/or RRM2 as compared to expression of same in normal PC is indicative of responsiveness to treatment with said proteasome inhibitor and said agent.

16. The method of claim 6, wherein said at least one agent is PPIA inhibitor.

17. The method of claim 6, wherein said PPIA inhibitor is cyclosporine A (CSa).

18. The method of claim 6, wherein said RRM2 inhibitor is Cladribine.

19. The method of claim 6, wherein said PPIA inhibitor is cyclosporine A (CSa) and said RRM2 inhibitor is Cladribine;
or wherein said proteasome inhibitor is Carfilzomib, wherein said PPIA inhibitor is cyclosporine A (CsA) and optionally comprising dexamethasone.
